(19) United States
(12) Patent Application Publication
(10) Pub. No.: US 2010/0204221 A1
(43) Pub. Date: Aug. 12, 2010

(54) Pyrrolopyrimidinyl axl kinase inhibitors

(76) Inventors: Hariprasad Vankayalapati, Draper, UT (US); Xiao-Hui Liu, Sandy, UT (US); William Merton Hewitt, Salt Lake City, UT (US); Eric Scott Gourley, Taylorsville, UT (US); Yong Xu, Midvale, UT (US); Bhasker Aavula, Pleasanton, CA (US)

Correspondence Address: SuperGen, Inc., 4140 Dublin Blvd., Suite 200, Dublin, CA, 94568, US

(21) Appl. No.: 12/658,433

(22) Filed: Feb. 8, 2010

Related U.S. Application Data
(60) Provisional application No. 61/207,292, filed on Feb. 9, 2009.

(51) Int. Cl. A61K 31/5377; C07D 487/04; A61K 31/519; C07D 413/14; C07D 495/04; A61P 35/00

(52) U.S. Cl. 514/234.2; 544/280; 514/265.1; 514/252.16; 544/117; 544/278

(57) ABSTRACT

Compounds represented by Formula (I):

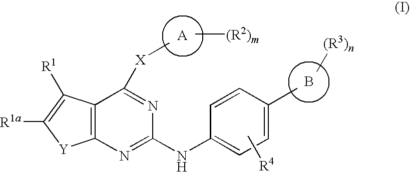

are useful in treating diseases, such as cancer, that are mediated and/or associated (at least in part) with Axl kinase. The compounds can be formulated as pharmaceutically acceptable compositions for administration to a subject in need thereof.

This application claims the benefit of U.S. Patent Application No. 61/207,292 filed Feb. 9, 2009.

### FIELD OF THE INVENTION

The present invention relates generally to fused 5,6 hetero ring compounds that inhibit protein kinase activity, and to compositions and methods related thereto. In particular, the present invention relates to 7H-pyrrolo[2,3-d]pyrimidin-4-yl amino compounds that inhibit protein kinase activity, such as Axl, useful in the treatment of cancer and hyperproliferative diseases.

### DESCRIPTION OF THE RELATED ART

Cancer (and other hyperproliferative diseases) is characterized by uncontrolled cell proliferation. This loss of the normal control of cell proliferation often appears as the result of genetic damage to cell pathways that control progress through the cell cycle. The cell cycle consists of DNA synthesis (S-phase), cell division or mitosis (M-phase), and non-synthetic periods referred to as gap 1 (G1) and gap 2 (G2). The M-phase is composed of mitosis and cytokinesis (separation into two cells). All steps in the cell cycle are controlled by an orderly cascade of protein phosphorylation. Several families of protein kinases are involved in carrying out these phosphorylation steps. In addition, the activity of many protein kinases increases in human tumors compared to normal tissue and such increased activity can be due to many factors, including i) increased levels of a kinase or ii) changes in expression of co-activators or inhibitory proteins.

Cells have proteins that govern the transition from one phase of the cell cycle to another. For example, the cyclins are a family of proteins whose concentrations increase and decrease throughout the cell cycle. The cyclins turn on, at the appropriate time, different cyclin-dependent protein kinases (CDKs) that phosphorylate substrates essential for progression through the cell cycle. Activity of specific CDKs at specific times is essential for both initiation and for coordinated progress through the cell cycle. For example, CDK1 is the most prominent cell cycle regulator that orchestrates M-phase activities. However, a number of other mitotic protein kinases that participate in M-phase have been identified, including members of the polo, aurora, and NIMA (Never-In-Mitosis-A) families, as well as kinases implicated in mitotic checkpoints, mitotic exit, and cytokinesis.

Axl is a receptor tyrosine kinase (ligand: Growth Arrest Specific protein 6, Gas6) which is unique in having two tandem immunoglobulin-like repeats and two fibronectin type III repeats, a feature common in cellular adhesion molecules. For this reason, it has a family of its own, the Axl/Ufo subfamily of tyrosine kinases. The expression of Axl/Gas6 has been shown in a number of human malignancies, including ovarian, melanoma, renal cell carcinoma, uterine leiomyoma, uterine endometrial cancer, thyroid carcinoma, gastric cancer, breast cancer, non-small cell lung cancer (NSCLC), chronic myelogenous leukemia (CML), acute myelogenous leukemia (AML), colorectal carcinoma, prostate cancer, various lymphomas, and esophageal cancer. The Axl proto-oncogene is thus an attractive and valuable target for the discovery and development of new therapeutic agents.

Axl is also implicated in the inflammation pathway, including rheumatoid arthritis. See, for example: K. O'Donnell et al., Am. J. Pathology, 154(4): 1171-1180 (1999); S. Hafizi et al., Int. J. Biochem. & Cell Biology, 37:2344-2356 (2005); and M. G. Melaragno et al., Circ. Res., 83:697-704 (1998). Thus, Axl inhibition would affect afflictions such as asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration, which are associated with cytokine activity. Axl inhibition also would be of benefit in the treatment of nonmalignant tumors such as, for example, Castleman's disease.

International Patent Publication No. WO 2007089768 describes 4-aryl-2-aminopyrimidines or 4-aryl-2-aminoalkylpyrimidines as JAK-2 modulators and their preparation, pharmaceutical compositions, and use in the treatment of diseases.

The compound

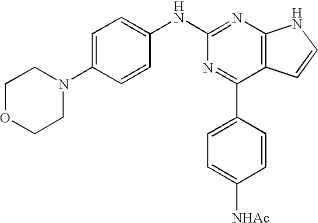

is known in certain compound libraries.

International Patent Publication No. WO 2006055351 describes atomic structure of natural quinine-responsive riboswitch useful for identifying compound, comprises atomic structure of hypoxanthine binding pocket. International Patent Publication No. WO 2004042029 describes composition useful for modulating expression of target nucleic acid, comprises first oligomer capable of hybridizing with target nucleic acid and second oligomer, and second oligomer.

Based on the Axl kinase's involvement in a number of human malignancies and in inflammation, there is a need for the design of specific and selective inhibitors for the treatment of cancer, inflammation, and other conditions mediated and/or associated with Axl kinase. The present invention fulfills these needs and offers other related advantages.

### BRIEF SUMMARY OF THE INVENTION

The present invention is generally directed to compounds having the following general Formula (I):

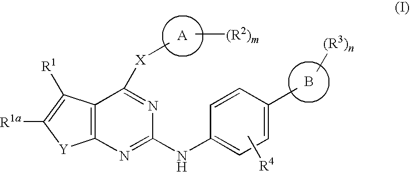

useful in treating diseases, such as cancer, that are mediated and/or associated (at least in part) with Axl kinase. The compounds can be formulated as pharmaceutically acceptable compositions for administration to a subject in need thereof.

The compounds of the present invention can also be used to treat or prevent asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration, which are associated with cytokine activity. The compounds of the present invention also would be of benefit in the treatment of nonmalignant tumors such as, for example, Castleman's disease.

These and other aspects of the invention will be apparent upon reference to the following detailed description. To that end, certain patent and other documents are cited herein to more specifically set forth various aspects of this invention. Each of these documents is hereby incorporated by reference in its entirety.

### DETAILED DESCRIPTION OF THE INVENTION

The present invention is generally directed to compounds having the following general structure according to Formula (I):

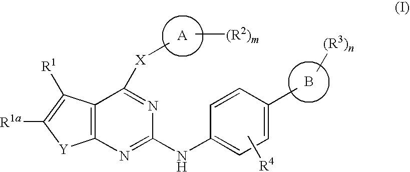

and pharmaceutically acceptable salts thereof, wherein:

X is —NH—, S, or a direct bond;

Y is —NH— or S;

A is aryl or hetaryl;

B is —O—C₁₋₄alkyl-N(C₀₋₄alkyl)(C₀₋₄alkyl), or C₁₋₄alkyl optionally substituted by —CN, or

B is hetcyclyl, —C(O)-hetcyclyl, —NH-hetcyclyl, or —O—C₀₋₄alkyl-hetcyclyl;

R^{1a} is C₀₋₄alkyl;

R¹ is halo, —CN, —OH, C₀₋₄alkyl, halo substituted C₁₋₄alkyl, —COOH, or —CONH₂;

R² in each instance independently is —CN, halo, C₀₋₄alkyl, —O—C₁₋₄alkyl, —O—C₁₋₄haloalkyl, or —N(R^{b})(R^{a}); or C₁₋₄alkyl optionally substituted by halo, —CN, —O—C₁₋₄alkyl, or —O—C₁₋₄haloalkyl; R^{a} and R^{b} each independently in each case is C₀₋₄alkyl, or —C(O)—C₃₋₆cycloalkyl;

R³ in each instance independently is —CN, C₀₋₄alkyl, halo, C₀₋₄alkyl-N(C₀₋₄alkyl)(C₃₋₄alkyl), C₃₋₈cycloalkyl, —S(O)₂—CH₃, or —C(O)—O—C₁₋₄alkyl-aryl; or C₁₋₄alkyl optionally substituted with 1-6 independent halo or OH substituents;

R⁴ is C₀₋₄alkyl, halo, or halo substituted C₁₋₄alkyl;

m is 0, 1, 2 or 3; and

n is 0, 1, 2, or 3; with the proviso that the compound is not

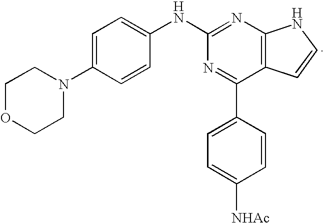

In an aspect of the invention, compounds of the present invention are described by Formula (I) and pharmaceutically acceptable salts thereof, wherein X is —NH— and the other variables are as defined above for Formula (I).

In an embodiment of this aspect, compounds of the present invention are described by Formula (I) and pharmaceutically acceptable salts thereof, wherein X is —NH—, Y is —NH—, and the other variables are as defined above for Formula (I).

In an embodiment of this aspect, compounds of the present invention are described by Formula (I) and pharmaceutically acceptable salts thereof, wherein X is —NH—, Y is —NH—, A is aryl, and the other variables are as defined above for Formula (I).

In another embodiment of this aspect, compounds of the present invention are described by Formula (I) and pharmaceutically acceptable salts thereof, wherein X is —NH—, Y is —NH—, A is aryl, B is C₁₋₄alkyl optionally substituted by —CN, and the other variables are as defined above for Formula (I).

In yet another embodiment of this aspect, compounds of the present invention are described by Formula (I) and pharmaceutically acceptable salts thereof, wherein X is —NH—, Y is —NH—, A is aryl, B is —C(O)-hetcyclyl, and the other variables are as defined above for Formula (I).

In still another embodiment of this aspect, compounds of the present invention are described by Formula (I) and pharmaceutically acceptable salts thereof, wherein X is —NH—, Y is —NH—, A is aryl, B is hetcyclyl, and the other variables are as defined above for Formula (I).

In another embodiment of this aspect, compounds of the present invention are described by Formula (I) and pharmaceutically acceptable salts thereof, wherein X is —NH—, Y is —NH—, A is aryl, B is —O—C₁₋₄alkyl-N(C₀₋₄alkyl)(C₀₋₄alkyl), and the other variables are as defined above for Formula (I).

In another embodiment of this aspect, compounds of the present invention are described by Formula (I) and pharmaceutically acceptable salts thereof, wherein X is —NH—, Y is —NH—, A is aryl, B is —NH-hetcyclyl, and the other variables are as defined above for Formula (I).

In another embodiment of this aspect, compounds of the present invention are described by Formula (I) and pharmaceutically acceptable salts thereof, wherein X is —NH—, Y is —NH—, A is aryl, B is —O—C₀₋₄alkyl-hetcyclyl, and the other variables are as defined above for Formula (I).

In yet another embodiment of this aspect, compounds of the present invention are described by Formula (I) and pharmaceutically acceptable salts thereof, wherein X is —NH—, Y is —NH—, A is phenyl, B is —C(O)-hetcyclyl, and the other variables are as defined above for Formula (I).

In still another embodiment of this aspect, compounds of the present invention are described by Formula (I) and pharmaceutically acceptable salts thereof, wherein X is —NH—, Y is —NH—, A is phenyl, B is hetcyclyl, and the other variables are as defined above for Formula (I).

In another embodiment of this aspect, compounds of the present invention are described by Formula (I) and pharmaceutically acceptable salts thereof, wherein X is —NH—, Y is —NH—, A is phenyl, B is C₁₋₄alkyl optionally substituted by —CN, and the other variables are as defined above for Formula (I).

In yet another embodiment of this aspect, compounds of the present invention are described by Formula (I) and pharmaceutically acceptable salts thereof, wherein X is —NH—, Y is —NH—, A is phenyl, B is —C(O)-hetcyclyl, R¹ is H, and the other variables are as defined above for Formula (I).

In still another embodiment of this aspect, compounds of the present invention are described by Formula (I) and pharmaceutically acceptable salts thereof, wherein X is —NH—, Y is —NH—, A is phenyl, B is hetcyclyl, R¹ is H, and the other variables are as defined above for Formula (I).

In another embodiment of this aspect, compounds of the present invention are described by Formula (I) and pharmaceutically acceptable salts thereof, wherein X is —NH—, Y is —NH—, A is phenyl, B is C₁₋₄alkyl optionally substituted by —CN, R¹ is H, and the other variables are as defined above for Formula (I).

In another embodiment of this aspect, compounds of the present invention are described by Formula (I) and pharmaceutically acceptable salts thereof, wherein X is —NH—, Y is —NH—, A is phenyl, B is —O—C₁₋₄alkyl-N(C₀₋₄alkyl)(C₀₋₄alkyl), and the other variables are as defined above for Formula (I).

In another embodiment of this aspect, compounds of the present invention are described by Formula (I) and pharmaceutically acceptable salts thereof, wherein X is —NH—, Y is —NH—, A is phenyl, B is —NH-hetcyclyl, and the other variables are as defined above for Formula (I).

In another embodiment of this aspect, compounds of the present invention are described by Formula (I) and pharmaceutically acceptable salts thereof, wherein X is —NH—, Y is —NH—, A is phenyl, B is —O—C₀₋₄alkyl-hetcyclyl, and the other variables are as defined above for Formula (I).

In another aspect of the invention, compounds of the present invention are described by Formula (I) and pharmaceutically acceptable salts thereof, wherein X is a direct bond, and the other variables are as defined above for Formula (I).

In an embodiment of this aspect, compounds of the present invention are described by Formula (I) and pharmaceutically acceptable salts thereof, wherein X is a direct bond, Y is —NH—, and the other variables are as defined above for Formula (I).

In an embodiment of this aspect, compounds of the present invention are described by Formula (I) and pharmaceutically acceptable salts thereof, wherein X is a direct bond, Y is —NH—, A is aryl, and the other variables are as defined above for Formula (I).

In another embodiment of this aspect, compounds of the present invention are described by Formula (I) and pharmaceutically acceptable salts thereof, wherein X is a direct bond, Y is —NH—, A is aryl, B is C₁₋₄alkyl optionally substituted by —CN, and the other variables are as defined above for Formula (I).

In yet another embodiment of this aspect, compounds of the present invention are described by Formula (I) and pharmaceutically acceptable salts thereof, wherein X is a direct bond, Y is —NH—, A is aryl, B is —C(O)-hetcyclyl, and the other variables are as defined above for Formula (I).

In still another embodiment of this aspect, compounds of the present invention are described by Formula (I) and pharmaceutically acceptable salts thereof, wherein X is a direct bond, Y is —NH—, A is aryl, B is hetcyclyl, and the other variables are as defined above for Formula (I).

In another embodiment of this aspect, compounds of the present invention are described by Formula (I) and pharmaceutically acceptable salts thereof, wherein X is a direct bond, Y is —NH—, A is aryl, B is —O—C₁₋₄alkyl-N(C₀₋₄alkyl)(C₀₋₄alkyl), and the other variables are as defined above for Formula (I).

In another embodiment of this aspect, compounds of the present invention are described by Formula (I) and pharmaceutically acceptable salts thereof, wherein X is a direct bond, Y is —NH—, A is aryl, B is —NH-hetcyclyl, and the other variables are as defined above for Formula (I).

In another embodiment of this aspect, compounds of the present invention are described by Formula (I) and pharmaceutically acceptable salts thereof, wherein X is a direct bond, Y is —NH—, A is aryl, B is —O—C₀₋₄alkyl-hetcyclyl, and the other variables are as defined above for Formula (I).

In yet another embodiment of this aspect, compounds of the present invention are described by Formula (I) and pharmaceutically acceptable salts thereof, wherein X is a direct bond, Y is —NH—, A is phenyl, B is —C(O)-hetcyclyl, and the other variables are as defined above for Formula (I).

In still another embodiment of this aspect, compounds of the present invention are described by Formula (I) and pharmaceutically acceptable salts thereof, wherein X is a direct bond, Y is —NH—, A is phenyl, B is hetcyclyl, and the other variables are as defined above for Formula (I).

In another embodiment of this aspect, compounds of the present invention are described by Formula (I) and pharmaceutically acceptable salts thereof, wherein X is a direct bond, Y is —NH—, A is phenyl, B is C₁₋₄alkyl optionally substituted by —CN, and the other variables are as defined above for Formula (I).

In yet another embodiment of this aspect, compounds of the present invention are described by Formula (I) and pharmaceutically acceptable salts thereof, wherein X is a direct bond, Y is —NH—, A is phenyl, B is —C(O)-hetcyclyl, R¹ is H, and the other variables are as defined above for Formula (I).

In still another embodiment of this aspect, compounds of the present invention are described by Formula (I) and pharmaceutically acceptable salts thereof, wherein X is a direct bond, Y is —NH—, A is phenyl, B is hetcyclyl, R¹ is H, and the other variables are as defined above for Formula (I).

In another embodiment of this aspect, compounds of the present invention are described by Formula (I) and pharmaceutically acceptable salts thereof, wherein X is a direct bond, Y is —NH—, A is phenyl, B is C₁₋₄alkyl optionally substituted by —CN, R¹ is H, and the other variables are as defined above for Formula (I).

In another embodiment of this aspect, compounds of the present invention are described by Formula (I) and pharmaceutically acceptable salts thereof, wherein X is a direct bond, Y is —NH—, A is phenyl, B is —O—C₁₋₄alkyl-N(C₀₋₄alkyl)(C₀₋₄alkyl), and the other variables are as defined above for Formula (I).

In another embodiment of this aspect, compounds of the present invention are described by Formula (I) and pharmaceutically acceptable salts thereof, wherein X is a direct bond, Y is —NH—, A is phenyl, B is —NH-hetcyclyl, and the other variables are as defined above for Formula (I).

In another embodiment of this aspect, compounds of the present invention are described by Formula (I) and pharmaceutically acceptable salts thereof, wherein X is a direct bond, Y is —S—, A is phenyl, B is —O—C₀₋₄alkyl-hetcyclyl, and the other variables are as defined above for Formula (I).

In an embodiment of this aspect, compounds of the present invention are described by Formula (I) and pharmaceutically acceptable salts thereof, wherein X is a direct bond, Y is —S—, and the other variables are as defined above for Formula (I).

In an embodiment of this aspect, compounds of the present invention are described by Formula (I) and pharmaceutically acceptable salts thereof, wherein X is a direct bond, Y is —S—, A is aryl, and the other variables are as defined above for Formula (I).

In another embodiment of this aspect, compounds of the present invention are described by Formula (I) and pharmaceutically acceptable salts thereof, wherein X is a direct bond, Y is —S—, A is aryl, B is C₁₋₄alkyl optionally substituted by —CN, and the other variables are as defined above for Formula (I).

In yet another embodiment of this aspect, compounds of the present invention are described by Formula (I) and pharmaceutically acceptable salts thereof, wherein X is a direct bond, Y is —S—, A is aryl, B is —C(O)-hetcyclyl, and the other variables are as defined above for Formula (I).

In still another embodiment of this aspect, compounds of the present invention are described by Formula (I) and pharmaceutically acceptable salts thereof, wherein X is a direct bond, Y is —S—, A is aryl, B is hetcyclyl, and the other variables are as defined above for Formula (I).

In another embodiment of this aspect, compounds of the present invention are described by Formula (I) and pharmaceutically acceptable salts thereof, wherein X is a direct bond, Y is —S—, A is aryl, B is —O—C₁₋₄alkyl-N(C₀₋₄alkyl)(C₀₋₄alkyl), and the other variables are as defined above for Formula (I).

In another embodiment of this aspect, compounds of the present invention are described by Formula (I) and pharmaceutically acceptable salts thereof, wherein X is a direct bond, Y is —S—, A is aryl, B is —NH-hetcyclyl, and the other variables are as defined above for Formula (I).

In another embodiment of this aspect, compounds of the present invention are described by Formula (I) and pharmaceutically acceptable salts thereof, wherein X is a direct bond, Y is —S—, A is aryl, B is —O—C₀₋₄alkyl-hetcyclyl, and the other variables are as defined above for Formula (I).

In yet another embodiment of this aspect, compounds of the present invention are described by Formula (I) and pharmaceutically acceptable salts thereof, wherein X is a direct bond, Y is —S—, A is phenyl, B is —C(O)-hetcyclyl, and the other variables are as defined above for Formula (I).

In still another embodiment of this aspect, compounds of the present invention are described by Formula (I) and pharmaceutically acceptable salts thereof, wherein X is a direct bond, Y is —S—, A is phenyl, B is hetcyclyl, and the other variables are as defined above for Formula (I).

In another embodiment of this aspect, compounds of the present invention are described by Formula (I) and pharmaceutically acceptable salts thereof, wherein X is a direct bond, Y is —S—, A is phenyl, B is C₁₋₄alkyl optionally substituted by —CN, and the other variables are as defined above for Formula (I).

In yet another embodiment of this aspect, compounds of the present invention are described by Formula (I) and pharmaceutically acceptable salts thereof, wherein X is a direct bond, Y is —S—, A is phenyl, B is —C(O)-hetcyclyl, R¹ is H, and the other variables are as defined above for Formula (I).

In still another embodiment of this aspect, compounds of the present invention are described by Formula (I) and pharmaceutically acceptable salts thereof, wherein X is a direct bond, Y is —S—, A is phenyl, B is hetcyclyl, R¹ is H, and the other variables are as defined above for Formula (I).

In another embodiment of this aspect, compounds of the present invention are described by Formula (I) and pharmaceutically acceptable salts thereof, wherein X is a direct bond, Y is —S—, A is phenyl, B is C₁₋₄alkyl optionally substituted by —CN, R¹ is H, and the other variables are as defined above for Formula (I).

In another embodiment of this aspect, compounds of the present invention are described by Formula (I) and pharmaceutically acceptable salts thereof, wherein X is a direct bond, Y is —S—, A is phenyl, B is —O—C₁₋₄alkyl-N(C₀₋₄alkyl)(C₀₋₄alkyl), and the other variables are as defined above for Formula (I).

In another embodiment of this aspect, compounds of the present invention are described by Formula (I) and pharmaceutically acceptable salts thereof, wherein X is a direct bond, Y is —S—, A is phenyl, B is —NH-hetcyclyl, and the other variables are as defined above for Formula (I).

In another embodiment of this aspect, compounds of the present invention are described by Formula (I) and pharmaceutically acceptable salts thereof, wherein X is a direct bond, Y is —S—, A is phenyl, B is —O—C₀₋₄alkyl-hetcyclyl, and the other variables are as defined above for Formula (I).

These compounds have utility over a broad range of therapeutic applications, and may be used to treat diseases, such as cancer, that are mediated and/or associated (at least in part) with Axl kinase. Accordingly, in one aspect of the invention, the compounds described herein are formulated as pharmaceutically acceptable compositions for administration to a subject in need thereof.

In another aspect, the invention provides methods for treating or preventing a Axl kinase-mediated disease, such as cancer, which method comprises administering to a patient in need of such a treatment a therapeutically effective amount of a compound described herein or a pharmaceutically acceptable composition comprising said compound.

Another aspect relates to inhibiting Axl kinase activity in a biological sample, which method comprises contacting the biological sample with a compound described herein, or a pharmaceutically acceptable composition comprising said compound.

Another aspect relates to a method of inhibiting Axl kinase activity in a patient, which method comprises administering to the patient a compound described herein or a pharmaceutically acceptable composition comprising said compound.

These and other aspects of the invention will be apparent upon reference to the following detailed description. To that end, certain patent and other documents are cited herein to more specifically set forth various aspects of this invention. Each of these documents is hereby incorporated by reference in its entirety.

Unless otherwise stated the following terms used in the specification and claims have the meanings discussed below:

“Alkyl” refers to a saturated straight or branched hydrocarbon radical of one to six carbon atoms, preferably one to four carbon atoms, e.g., methyl, ethyl, propyl, 2-propyl, n-butyl, iso-butyl, tert-butyl, pentyl, hexyl, and the like, preferably methyl, ethyl, propyl, or 2-propyl. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Cyclic alkyls are referred to herein as a “cycloalkyl.”

Unsaturated alkyls contain at least one double or triple bond between adjacent carbon atoms (referred to as an “alkenyl” or “alkynyl”, respectively.) Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“C₀₋₄Alkyl” refers to an alkyl with 0, 1, 2, 3, or 4 carbon atoms. C₀₋₄alkyl with 0 carbon atoms is a hydrogen atom when terminal and is a direct bond when linking.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like, preferably methylene, ethylene, or propylene.

“Cycloalkyl” refers to a saturated cyclic hydrocarbon radical of three to eight carbon atoms, e.g., cyclopropyl, cyclobutyl, cyclopentyl or cyclohexyl.

“Alkoxy” means a radical —ORₐ where Rₐ is an alkyl as defined above, e.g., methoxy, ethoxy, propoxy, butoxy and the like.

“Halo” means fluoro, chloro, bromo, or iodo, preferably fluoro and chloro.

“Haloalkyl” means alkyl substituted with one or more, preferably one, two or three, same or different halo atoms, e.g., —CH₂C1, —CF₃, —CH₂CF₃, —CH₂CCl₃, and the like.

“Haloalkoxy” means a radical —OR_{b} where R_{b} is an haloalkyl as defined above, e.g., trifluoromethoxy, trichloroethoxy, 2,2-dichloropropoxy, and the like.

“Acyl” means a radical —C(O)R_{c}, where R_{c} is hydrogen, alkyl, or haloalkyl as defined herein, e.g., formyl, acetyl, trifluoroacetyl, butanoyl, and the like.

“Aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The aryl group may be substituted or unsubstituted. Unless specifically stated otherwise, “substituted aryl” refers to the aryl group being substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, phenoxy, heteroaryl, heteroaryloxy, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Heteroaryl” refers to a monocyclic or fused ring (i.e., rings which share an adjacent pair of atoms) group of 5 to 12 ring atoms containing one, two, three or four ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, and carbazole. The heteroaryl group may be substituted or unsubstituted. Unless specifically stated otherwise, “substituted heteroaryl” refers to the heteroaryl group being substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Carbocycle” refers to a saturated, unsaturated or aromatic ring system having 3 to 14 ring carbon atoms. The term “carbocycle”, whether saturated or partially unsaturated, also refers to rings that are optionally substituted. The term “carbocycle” includes aryl. The term “carbocycle” also includes aliphatic rings that are fused to one or more aromatic or nonaromatic rings, such as in a decahydronaphthyl or tetrahydronaphthyl, where the radical or point of attachment is on the aliphatic ring. The carbocycle group may be substituted or unsubstituted. Unless specifically stated otherwise, “substituted carbocyle” refers to the carbocycle group being substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Heterocycle” refers to a saturated, unsaturated or aromatic cyclic ring system having 3 to 14 ring atoms in which one, two or three ring atoms are heteroatoms selected from N, O, or S(O)ₘ (where m is an integer from 0 to 2), the remaining ring atoms being C, where one or two C atoms may optionally be replaced by a carbonyl group. The term “heterocycle” includes heteroaryl. Unless specifically stated otherwise, “substituted heterocyclyl” refers to the heterocyclyl ring being substituted independently with one or more, preferably one, two, or three substituents selected from alkyl (wherein the alkyl may be optionally substituted with one or two substituents), haloalkyl, cycloalkylamino, cycloalkylalkyl, cycloalkylaminoalkyl, cycloalkylalkylaminoalkyl, cyanoalkyl, halo, nitro, cyano, hydroxy, alkoxy, amino, alkylamino, dialkylamino, hydroxyalkyl, carboxyalkyl, aminoalkyl, alkylaminoalkyl, dialkylaminoalkyl, aralkyl, heteroaralkyl, aryl, heteroaryl, carbocycle, heterocycle (wherein the aryl, heteroaryl, carbocycle or heterocycle may be optionally substituted), aralkyl, heteroaralkyl, saturated or unsaturated heterocycloamino, saturated or unsaturated heterocycloaminoalkyl, and COR_{d} (where R_{d} is alkyl). More specifically the term heterocyclyl includes, but is not limited to, tetrahydropyranyl, 2,2-dimethyl-1,3-dioxolane, piperidino, N-methylpiperidin-3-yl, piperazino, N-methylpyrrolidin-3-yl, pyrrolidino, morpholino, 4-cyclopropylmethylpiperazino, thiomorpholino, thiomorpholino-1-oxide, thiomorpholino-1,1-dioxide, 4-ethyloxycarbonylpiperazino, 3-oxopiperazino, 2-imidazolidone, 2-pyrrolidinone, 2-oxohomopiperazino, tetrahydropyrimidin-2-one, and the derivatives thereof. In certain embodiments, the heterocycle group is optionally substituted with one or two substituents independently selected from halo, alkyl, alkyl substituted with carboxy, ester, hydroxy, alkylamino, saturated or unsaturated heterocycloamino, saturated or unsaturated heterocycloaminoalkyl, or dialkylamino.

“Optional” or “optionally” means that the subsequently described event or circumstance may but need not occur, and that the description includes instances where the event or circumstance occurs and instances in which it does not. For example, “heterocyclic group optionally substituted with an alkyl group” means that the alkyl may but need not be present, and the description includes situations where the heterocycle group is substituted with an alkyl group and situations where the heterocycle group is not substituted with the alkyl group.

Lastly, unless specifically stated otherwise, the term “substituted” as used herein means any of the above groups (e.g., alkyl, aryl, heteroaryl, carbocycle, heterocycle, etc.) wherein at least one hydrogen atom is replaced with a substituent. In the case of an oxo substituent (“═O”) two hydrogen atoms are replaced. “Substituents” within the context of this invention include halogen, hydroxy, oxo, cyano, nitro, amino, alkylamino, dialkylamino, alkyl, alkoxy, thioalkyl, haloalkyl (e.g., —CF₃), hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₑR_{f}, —NRₑC(═O)R_{f}, —NRₑC(═O)NRₑR_{f}, —NRₑC(═O)OR_{f}—NRₑSO₂R_{f}, —ORₑ, —C(═O)Rₑ—C(═O)ORₑ, —C(═O)NRₑR_{f}, —OC(═O)NRₑR_{f}, —SH, —SRₑ, —SORₑ, —S(═O)₂Rₑ, —OS(═O)₂Rₑ, —S(═O)₂ORₑ, wherein Rₑ and R_{f} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog (Calm, R., Ingold, C., and Prelog, V. Angew. Chem. 78:413-47, 1966; Angew. Chem. Internat. Ed. Eng. 5:385-415, 511, 1966), or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Ch. 4 of ADVANCED ORGANIC CHEMISTRY, 4^{th} edition, March, J., John Wiley and Sons, New York City, 1992).

The compounds of the present invention may exhibit the phenomena of tautomerism and structural isomerism. This invention encompasses any tautomeric or structural isomeric form and mixtures thereof which possess the ability to modulate Axl kinase activity and is not limited to, any one tautomeric or structural isomeric form.

It is contemplated that a compound of the present invention would be metabolized by enzymes in the body of the organism such as human being to generate a metabolite that can modulate the activity of the protein kinases. Such metabolites are within the scope of the present invention.

A compound of the present invention or a pharmaceutically acceptable salt thereof, can be administered as such to a human patient or can be administered in pharmaceutical compositions in which the foregoing materials are mixed with suitable carriers or excipient(s). Techniques for formulation and administration of drugs may be found, for example, in REMINGTON'S PHARMACOLOGICAL SCIENCES, Mack Publishing Co., Easton, Pa., latest edition.

A “pharmaceutical composition” refers to a mixture of one or more of the compounds described herein, or pharmaceutically acceptable salts or prodrugs thereof, with other chemical components, such as pharmaceutically acceptable excipients. The purpose of a pharmaceutical composition is to facilitate administration of a compound to an organism.

“Pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

“Pharmaceutically acceptable salt” refers to those salts which retain the biological effectiveness and properties of the parent compound. Such salts may include: (1) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D)- or (L)-malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The compound of the present invention may also act, or be designed to act, as a prodrug. A “prodrug” refers to an agent, which is converted into the parent drug in vivo. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. An example, without limitation, of a prodrug would be a compound of the present invention, which is, administered as an ester (the “prodrug”), phosphate, amide, carbamate, or urea.

“Therapeutically effective amount” refers to that amount of the compound being administered which will relieve to some extent one or more of the symptoms of the disorder being treated. In reference to the treatment of cancer, a therapeutically effective amount refers to that amount which has the effect of: (1) reducing the size of the tumor; (2) inhibiting tumor metastasis; (3) inhibiting tumor growth; and/or (4) relieving one or more symptoms associated with the cancer.

The term “protein kinase-mediated condition” or “disease”, as used herein, means any disease or other deleterious condition in which a protein kinase is known to play a role. The term “protein kinase-mediated condition” or “disease” also means those diseases or conditions that are alleviated by treatment with a protein kinase inhibitor. Such conditions include, without limitation, cancer and other hyperproliferative disorders. In certain embodiments, the cancer is a cancer of colon, breast, stomach, prostate, pancreas, or ovarian tissue.

The term “Axl kinase-mediated condition” or “disease”, as used herein, means any disease or other deleterious condition in which Axl kinase is overexpressed, overactive and/or is known to play a role. The term “Axl kinase-mediated condition” also means those diseases or conditions that are alleviated by treatment with an Axl kinase inhibitor.

As used herein, “administer” or “administration” refers to the delivery of an inventive compound or of a pharmaceutically acceptable salt thereof or of a pharmaceutical composition containing an inventive compound or a pharmaceutically acceptable salt thereof of this invention to an organism for the purpose of prevention or treatment of a protein kinase-related disorder.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. In certain embodiments, the preferred routes of administration are oral and intravenous. Alternatively, one may administer the compound in a local rather than systemic manner, for example, via injection of the compound directly into a solid tumor, often in a depot or sustained release formulation. Furthermore, one may administer the drug in a targeted drug delivery system, for example, in a liposome coated with tumor-specific antibody. In this way, the liposomes may be targeted to and taken up selectively by the tumor.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in any conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also. Pharmaceutical compositions which may also be used include hard gelatin capsules. The capsules or pills may be packaged into brown glass or plastic bottles to protect the active compound from light. The containers containing the active compound capsule formulation are preferably stored at controlled room temperature (15-30° C.).

For administration by inhalation, the compounds for use according to the present invention may be conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

A non-limiting example of a pharmaceutical carrier for the hydrophobic compounds of the invention is a cosolvent system comprising benzyl alcohol, a nonpolar surfactant, a water-miscible organic polymer and an aqueous phase such as the VPD cosolvent system. VPD is a solution of 3% w/v benzyl alcohol, 8% w/v of the nonpolar surfactant polysorbate 80, and 65% w/v polyethylene glycol 300, made up to volume in absolute ethanol. The VPD cosolvent system (VPD:D5W) consists of VPD diluted 1:1 with a 5% dextrose in water solution. This cosolvent system dissolves hydrophobic compounds well, and itself produces low toxicity upon systemic administration. Naturally, the proportions of such a cosolvent system may be varied considerably without destroying its solubility and toxicity characteristics. Furthermore, the identity of the cosolvent components may be varied: for example, other low-toxicity nonpolar surfactants may be used instead of polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the protein kinase-modulating compounds of the invention may be provided as physiologically acceptable salts wherein the claimed compound may form the negatively or the positively charged species. Examples of salts in which the compound forms the positively charged moiety include, without limitation, quaternary ammonium (defined elsewhere herein), salts such as the hydrochloride, sulfate, carbonate, lactate, tartrate, malate, maleate, succinate wherein the nitrogen atom of the quaternary ammonium group is a nitrogen of the selected compound of this invention which has reacted with the appropriate acid. Salts in which a compound of this invention forms the negatively charged species include, without limitation, the sodium, potassium, calcium and magnesium salts formed by the reaction of a carboxylic acid group in the compound with an appropriate base (e.g. sodium hydroxide (NaOH), potassium hydroxide (KOH), calcium hydroxide (Ca(OH)₂), etc.).

Pharmaceutical compositions suitable for use in the present invention include compositions wherein the active ingredients are contained in an amount sufficient to achieve the intended purpose, e.g., the modulation of protein kinase activity and/or the treatment or prevention of a protein kinase-related disorder.

More specifically, a therapeutically effective amount means an amount of compound effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of the protein kinase activity). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀ (both of which are discussed elsewhere herein) for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See, e.g., GOODMAN & GILMAN'S THE PHARMACOLOGICAL BASIS OF THERAPEUTICS, Ch. 3, 9^{th} ed., Ed. by Hardman, J., and Limbard, L., McGraw-Hill, New York City, 1996, p. 46.)

Dosage amount and interval may be adjusted individually to provide plasma levels of the active species which are sufficient to maintain the kinase modulating effects. These plasma levels are referred to as minimal effective concentrations (MECs). The MEC will vary for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%.

At present, the therapeutically effective amounts of compounds of the present invention may range from approximately 2.5 mg/m² to 1500 mg/m² per day. Additional illustrative amounts range from 0.2-1000 mg/qid, 2-500 mg/qid, and 20-250 mg/qid.

In cases of local administration or selective uptake, the effective local concentration of the drug may not be related to plasma concentration, and other procedures known in the art may be employed to determine the correct dosage amount and interval.

The amount of a composition administered will, of course, be dependent on the subject being treated, the severity of the affliction, the manner of administration, the judgment of the prescribing physician, etc.

The compositions may, if desired, be presented in a pack or dispenser device, such as an FDA approved kit, which may contain one or more unit dosage forms containing the active ingredient. The pack may for example comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

As mentioned above, the compounds and compositions of the invention will find utility in a broad range of diseases and conditions mediated by protein kinases, including diseases and conditions mediated by Axl kinase. Such diseases may include by way of example and not limitation, cancers such as lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

The inventive compound can be used in combination with one or more other chemotherapeutic agents. The dosage of the inventive compounds may be adjusted for any drug-drug reaction. In one embodiment, the chemotherapeutic agent is selected from the group consisting of mitotic inhibitors, alkylating agents, anti-metabolites, cell cycle inhibitors, enzymes, topoisomerase inhibitors such as CAMPTOSAR (irinotecan), biological response modifiers, anti-hormones, antiangiogenic agents such as MMP-2, MMP-9 and COX-2 inhibitors, anti-androgens, platinum coordination complexes (cisplatin, etc.), substituted ureas such as hydroxyurea; methylhydrazine derivatives, e.g., procarbazine; adrenocortical suppressants, e.g., mitotane, aminoglutethimide, hormone and hormone antagonists such as the adrenocorticosteriods (e.g., prednisone), progestins (e.g., hydroxyprogesterone caproate), estrogens (e.g., diethylstilbesterol), antiestrogens such as tamoxifen, androgens, e.g., testosterone propionate, and aromatase inhibitors, such as anastrozole, and AROMASIN (exemestane).

Examples of alkylating agents that the above method can be carried out in combination with include, without limitation, fluorouracil (5-FU) alone or in further combination with leukovorin; other pyrimidine analogs such as UFT, capecitabine, gemcitabine and cytarabine, the alkyl sulfonates, e.g., busulfan (used in the treatment of chronic granulocytic leukemia), improsulfan and piposulfan; aziridines, e.g., benzodepa, carboquone, meturedepa and uredepa; ethyleneimines and methylmelamines, e.g., altretamine, triethylenemelamine, triethylenephosphoramide, triethylenethiophosphoramide and trimethylolmelamine; and the nitrogen mustards, e.g., chlorambucil (used in the treatment of chronic lymphocytic leukemia, primary macroglobulinemia and non-Hodgkin's lymphoma), cyclophosphamide (used in the treatment of Hodgkin's disease, multiple myeloma, neuroblastoma, breast cancer, ovarian cancer, lung cancer, Wilm's tumor and rhabdomyosarcoma), estramustine, ifosfamide, novembrichin, prednimustine and uracil mustard (used in the treatment of primary thrombocytosis, non-Hodgkin's lymphoma, Hodgkin's disease and ovarian cancer); and triazines, e.g., dacarbazine (used in the treatment of soft tissue sarcoma).

Examples of antimetabolite chemotherapeutic agents that the above method can be carried out in combination with include, without limitation, folic acid analogs, e.g., methotrexate (used in the treatment of acute lymphocytic leukemia, choriocarcinoma, mycosis fungiodes, breast cancer, head and neck cancer and osteogenic sarcoma) and pteropterin; and the purine analogs such as mercaptopurine and thioguanine which find use in the treatment of acute granulocytic, acute lymphocytic and chronic granulocytic leukemias.

Examples of natural product-based chemotherapeutic agents that the above method can be carried out in combination with include, without limitation, the vinca alkaloids, e.g., vinblastine (used in the treatment of breast and testicular cancer), vincristine and vindesine; the epipodophyllotoxins, e.g., etoposide and teniposide, both of which are useful in the treatment of testicular cancer and Kaposi's sarcoma; the antibiotic chemotherapeutic agents, e.g., daunorubicin, doxorubicin, epirubicin, mitomycin (used to treat stomach, cervix, colon, breast, bladder and pancreatic cancer), dactinomycin, temozolomide, plicamycin, bleomycin (used in the treatment of skin, esophagus and genitourinary tract cancer); and the enzymatic chemotherapeutic agents such as L-asparaginase.

Examples of useful COX-II inhibitors include VIOXX, CELEBREX (celecoxib), valdecoxib, paracoxib, rofecoxib, and Cox 189.

Examples of useful matrix metalloproteinase inhibitors are described in WO 96/33172, WO 96/27583, European Patent Application No. 97304971.1, European Patent Application No. 99308617.2, WO 98/07697, WO 98/03516, WO 98/34918, WO 98/34915, WO 98/33768, WO 98/30566, European Patent Publication 606,046, European Patent Publication 931,788, WO 90/05719, WO 99/52910, WO 99/52889, WO 99/29667, PCT International Application No. PCT/IB98/01113, European Patent Application No. 99302232.1, Great Britain patent application number 9912961.1, U.S. Pat. No. 5,863,949, U.S. Pat. No. 5,861,510, and European Patent Publication 780,386, all of which are incorporated herein in their entireties by reference. Preferred MMP-2 and MMP-9 inhibitors are those that have little or no activity inhibiting MMP-1. More preferred are those that selectively inhibit MMP-2 and/or MMP-9 relative to the other matrix-metalloproteinases (i.e., MMP-1, MMP-3, MMP-4, MMP-5, MMP-6, MMP-7, MMP-8, MMP-10, MMP-11, MMP-12, and MMP-13).

Some specific examples of MMP inhibitors useful in the present invention are AG-3340, RO 32-3555, RS 13-0830, and compounds selected from: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; (R) 3-[4-(4-chloro-phenoxy)-benzenesulfonylamino]-tetrahydro-pyran-3-carboxylic acid hydroxyamide; (2R,3R) 1-[4-(4-fluoro-2-methylbenzyloxy)-benzenesulfonyl]-3-hydroxy-3-methyl-piperidine-2-carboxylic acid hydroxyamide; 3-[[(4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1]octane-3-carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of these compounds.

Other anti-angiogenesis agents, other COX-II inhibitors and other MMP inhibitors, can also be used in the present invention.

An inventive compound can also be used with other signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, such as HERCEPTIN (Genentech, Inc., South San Francisco, Calif.). EGFR inhibitors are described in, for example in WO 95/19970, WO 98/14451, WO 98/02434, and U.S. Pat. No. 5,747,498, and such substances can be used in the present invention as described herein.

EGFR-inhibiting agents include, but are not limited to, the monoclonal antibodies C225 and anti-EGFR 22Mab (ImClone Systems, Inc., New York, N.Y.), the compounds erlotinib (OSI Pharmaceuticals, Inc., Melville, N.Y.), ZD-1839 (AstraZeneca), BIBX-1382 (Boehringer Ingelheim), MDX-447 (Medarex Inc., Annandale, N.J.), and OLX-103 (Merck & Co., Whitehouse Station, N.J.), and EGF fusion toxin (Seragen Inc., Hopkinton, Mass.).

These and other EGFR-inhibiting agents can be used in the present invention. VEGF inhibitors, for example SU-5416 and SU-6668 (Sugen Inc., South San Francisco, Calif.), can also be combined with an inventive compound. VEGF inhibitors are described in, for example, WO 01/60814 A3, WO 99/24440, PCT International Application PCT/IB99/00797, WO 95/21613, WO 99/61422, U.S. Pat. No. 5,834,504, WO 01/60814, WO 98/50356, U.S. Pat. No. 5,883,113, U.S. Pat. No. 5,886,020, U.S. Pat. No. 5,792,783, WO 99/10349, WO 97/32856, WO 97/22596, WO 98/54093, WO 98/02438, WO 99/16755, and WO 98/02437, all of which are incorporated herein in their entireties by reference. Other examples of some specific VEGF inhibitors useful in the present invention are IM862 (Cytran Inc., Kirkland, Wash.); anti-VEGF monoclonal antibody of Genentech, Inc.; and angiozyme, a synthetic ribozyme from Ribozyme (Boulder, Colo.) and Chiron (Emeryville, Calif.). These and other VEGF inhibitors can be used in the present invention as described herein. Further, pErbB2 receptor inhibitors, such as GW-282974 (Glaxo Wellcome plc), and the monoclonal antibodies AR-209 (Aronex Pharmaceuticals Inc., The Woodlands, Tex.) and 2B-1 (Chiron), can furthermore be combined with an inventive compound, for example, those indicated in WO 98/02434, WO 99/35146, WO 99/35132, WO 98/02437, WO 97/13760, WO 95/19970, U.S. Pat. No. 5,587,458, and U.S. Pat. No. 5,877,305, which are all hereby incorporated herein in their entireties by reference. ErbB2 receptor inhibitors useful in the present invention are also described in U.S. Pat. No. 6,284,764, incorporated in its entirety herein by reference. The erbB2 receptor inhibitor compounds and substance described in the aforementioned PCT applications, U.S. patents, and U.S. provisional applications, as well as other compounds and substances that inhibit the erbB2 receptor, can be used with an inventive compound, in accordance with the present invention.

An inventive compound can also be used with other agents useful in treating cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase inhibitors described in the references cited in the “Background” section, of U.S. Pat. No. 6,258,824 B1.

The above method can also be carried out in combination with radiation therapy, wherein the amount of an inventive compound in combination with the radiation therapy is effective in treating the above diseases. Techniques for administering radiation therapy are known in the art, and these techniques can be used in the combination therapy described herein. The administration of the compound of the invention in this combination therapy can be determined as described herein.

As mentioned above, the compounds and compositions of the invention will find utility in a broad range of diseases and conditions mediated by Axl kinase. Such diseases may include by way of example and not limitation, Castleman's disease, atherosclerosis, coronary artery disease, peripheral edema, peripheral vascular disease, glaucoma, and wet or dry age-related macular degeneration (AMD), asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration.

Diseases or conditions of humans or other species which can be treated with inhibitors of cytokine or chemokine receptor function, include, but are not limited to: inflammatory or allergic diseases and conditions, including respiratory allergic diseases such as asthma, particularly bronchial asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersentitivity, interstitial lung diseases (ILD) (e.g., idiopathic pulmonary fibrosis, or ILD associated with rheumatoid-arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fasciitis; cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis.

Diseases or conditions of humans or other species which can be treated with modulators of chemokine receptor function, include, but are not limited to: immunosuppression, such as that in individuals with immunodeficiency syndromes such as AIDS or other viral infections, individuals undergoing radiation therapy, chemotherapy, therapy for autoimmune disease or drug therapy (e.g., corticosteroid therapy), which causes immunosuppression; immunosuppression due to congenital deficiency in receptor function or other causes; and infections diseases, such as parasitic diseases, including, but not limited to helminth infections, such as nematodes (round worms), (Trichuriasis, Enterobiasis, Ascariasis, Hookworm, Strongyloidiasis, Trichinosis, filariasis), trematodes (flukes) (Schistosomiasis, Clonorchiasis), cestodes (tape worms) (Echinococcosis, Taeniasis saginata, Cysticercosis), visceral worms, visceral larva migraines (e.g., Toxocara), eosinophilic gastroenteritis (e.g., Anisaki sp., Phocanema sp.), and cutaneous larva migraines (Ancylostona braziliense, Ancylostoma caninum). In addition, treatment of the aforementioned inflammatory, allergic and autoimmune diseases can also be contemplated for promoters of chemokine receptor function if one contemplates the delivery of sufficient compound to cause the loss of receptor expression on cells through the induction of chemokine receptor internalization or delivery of compound in a manner that results in the misdirection of the migration of cells.

The methods of the present invention are accordingly useful in the prevention and treatment of a wide variety of inflammatory and immunoregulatory disorders and diseases, allergic conditions, atopic conditions, as well as autoimmune pathologies. In a specific embodiment, the present invention is directed to the use of the subject compounds for the prevention or treatment of autoimmune diseases, such as rheumatoid arthritis or psoriatic arthritis.

The subject treated in the present methods is a mammal, preferably a human being, male or female, in whom modulation of cytokine receptor activity is desired. “Modulation” as used herein is intended to encompass antagonism, agonism, partial antagonism, inverse agonism and/or partial agonism. In a preferred aspect of the present invention, modulation refers to antagonism of cytokine receptor activity. The term “therapeutically effective amount” means the amount of the subject compound that will elicit the biological or medical response of a tissue, system, animal or human that is being sought by the researcher, veterinarian, medical doctor or other clinician.

The inventive compound can be used in combination with one or more other chemotherapeutic agents. The dosage of the inventive compounds may be adjusted for any drug-drug reaction. Combined therapy to modulate chemokine receptor activity and thereby prevent and treat inflammatory and immunoregulatory disorders and diseases, including asthma and allergic diseases, as well as autoimmune pathologies such as rheumatoid arthritis and atherosclerosis, and those pathologies noted above is illustrated by the combination of the compounds of this invention and other compounds which are known for such utilities.

For example, in the treatment or prevention of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codeine, embrel, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

Examples of other active ingredients that may be combined with a compound of the present invention, either administered separately or in the same pharmaceutical compositions, include, but are not limited to: (a) VLA-4 antagonists such as those described in U.S. Pat. No. 5,510,332, WO95/15973, WO96/01644, WO96/06108, WO96/20216, WO96/22966, WO96/31206, WO96/40781, WO97/03094, WO97/02289, WO98/42656, WO98/53814, W98/53817, WO98/53818, WO98/54207, and WO98/58902; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (e) non-steroidal anti-asthmatics such as .beta.2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumnic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CCR-1, CCR-2, CCR-3, CXCR-3 and CCR-5; (j) cholesterol lowering agents such as HMG-CoA reductase inhibitors (lovastatin, simvastatin and pravastatin, fluvastatin, atorvastatin, and other statins), sequestrants (cholestyramine and colestipol), cholesterol absorption inhibitors (ezetimibe), nicotinic acid, fenofibric acid derivatives (gemfibrozil, clofibrat, fenofibrate and benzafibrate), and probucol; (k) anti-diabetic agents such as insulin, sulfonylureas, biguanides (metformin), .alpha.-glucosidase inhibitors (acarbose) and glitazones (troglitazone and pioglitazone); (1) preparations of interferon beta (interferon beta-1.alpha., interferon beta-1.beta.); (m) other compounds such as 5-aminosalicylic acid and prodrugs thereof, antimetabolites such as azathioprine and 6-mercaptopurine, and cytotoxic cancer chemotherapeutic agents.

Examples of useful COX-II inhibitors include VIOXX, CELEBREX (celecoxib), valdecoxib, paracoxib, rofecoxib, and Cox 189.

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The invention will be further understood upon consideration of the following non-limiting Examples.

### EXAMPLES

### Protocols & Data for Axl Kinase

(Invitrogen Corporation, Carlsbad, Calif.) LANTHASCREEN™ Kinase Assay

The premise of Time-Resolved Fluorescence Energy Transfer (TR-FRET) is the exchange of energy from a lanthanide chelate molecule such as terbium attached to the PY-20 antibody acting as a donor molecule. Upon antibody binding to phosphorylated tyrosine residues on the Fluorescein-poly-GT substrate, an acceptor molecule covalently attached to the substrate receives the energy transfer from the donor as the two molecules are brought into close proximity and the donor molecule undergoes excitation from a flashlamp. The energy transfer is measured as the emissions of the acceptor molecule increases while the emissions of the donor molecule decreases. In this manner drug potency against Axl kinase activity can be deduced.

TR-FRET utilizes the long excited-state of the terbium molecule to delay measuring the energy transfer long enough for background light scatter and/or fluorescence to dissipate while simultaneously avoiding direct excitation from the flashlamp source. Thus, TR-FRET is able to overcome some of the limitations of the standard FRET assay.

Optimization of the assay for Kₘ of ATP (Cat# PV3227 Invitrogen Corporation, Carlsbad, Calif.) was done by Vatsala via a series of serial dilutions of Axl kinase enzyme at 1 mM ATP. The EC₈₀ was determined as optimal ATP concentration for the assay. The EC₈₀ was 20 μM.

Optimization of concentration of Axl kinase enzyme (Cat# PV3971 Invitrogen Corporation, Carlsbad, Calif.) was done by a series of serial dilutions of enzyme at 20 μM ATP to determine the EC₈₀. This was done by Vatsala and the EC₈₀ was determined to be 103 ng/mL of enzyme.

Inhibitor IC₅₀ Determination Steps:
1) Create a master series of serial dilutions at 100× the final working concentration of inhibitor desired in the assay—for Axl that was at 1:2 dilution beginning at 10 mM down to 610 nM at 100× final conc. The final working conc. of inhibitors in all assays was 100 μM down to 6.1 nM. This is done in 25 μL of 100% DMSO in PCR strip tubes.
2) From the master dilutions in step 1, intermediate dilutions are made in a 96-well plate in the aqueous 1× Kinase Buffer (Cat# PV3189 5× Invitrogen Corporation, Carlsbad, Calif.). This was done by adding 96 μL of kinase buffer (KB) per well to a 96-well plate and mixing in 4 μL of inhibitor by vortex shaking.
3) From the intermediate dilutions in step 2, remove 2.5 μL of inhibitor from each well and add it to triplicate technical replicate wells of a 384-well plate.
4) Prepare a 4 μg/mL dilution of Axl kinase in KB from a 330 μg/mL stock of enzyme.
5) From the dilution in step 4 make a 412 ng/mL (4× of 103 ng/mL optimal enzyme conc.) dilution of enzyme in KB and add 2.5 μL of this dilution to each well of the 384-well plate containing inhibitor.
6) Prepare the appropriate volume of a solution in KB containing 20 μM ATP from a 10 mM stock and 0.4 μM Fluorescein-Poly GT substrate (Cat# PV3610 Invitrogen Corporation, Carlsbad, Calif.) from a 30 μM stock. Add 5 uL of this mix to each well of the 384-well plate to begin the reaction. Mix on a vortex plate shaker for 30 s at 80 rpm. Seal and incubate for 1 hr at RT.
7) Prepare the appropriate volume of a solution in TR-FRET Dilution Buffer (Cat# PV3574 Invitrogen Corporation, Carlsbad, Calif.) containing 20 mM EDTA Kinase Quench Buffer (Cat# P2832 Invitrogen Corporation, Carlsbad, Calif.) from a 500 mM stock and 4 nM LanthaScreen™ Tb-PY20 antibody (Cat# PV3553 Invitrogen Corporation, Carlsbad, Calif.) from a 6700 nM stock. Add 10 μL per well of the 384-well plate containing reaction from step 6. Seal and incubate for 30 min at rt.
8) Read on the Wallac Envision™ PerkinElmer 2103 Multilable Reader (PerkinElmer Waltham, Mass.) with a 340 nm excitation filter and both a 495 nm emission filter for the terbium donor signal and a 520 nm emission filter for the fluorescein acceptor signal detection.
9) Analysis of data done on Excel (Microsoft Corporation Redmond, Wash.) by calculating the mean emission and standard error for each technical replicate. The Reduction in Emission was calculated by the subtracting the mean emission value of the ATP only control from each mean emission value in the assay. Those values were used to calculate the Percent Activity by dividing each reduction emission value by the reduction emission value for the no inhibitor control. A scatter graph with a fitted line was created to show the relationship between the concentration of the drug and the percent activity of the Axl kinase enzyme. Each point was fitted with standard error bars and the IC₅₀ calculated. The IC₅₀ was calculated by graphing the concentration of drug points and the corresponding percent activity points directly above and below the 50% value. The equation of the line was used to solve for “x” in the equation which represents the IC₅₀ value for each drug. The data were also calculated on GrapPad Prism 5 software (GraphPad Software La Jolla, Calif.).

The EXAMPLES of the present invention displayed IC₅₀ results in the above TR-FRET assay ranging from about 4.08 μM to about 0.014 μM. It is advantageous that the IC₅₀ results be less than 3.0 μM. It is more advantageous that the IC₅₀ results be less than 1.0 μM. It is still more advantageous that the IC₅₀ be less than 0.1 μM.

Cell Titer-Glo Assay (Promega Corporation, Madison, Wis.)

Cell culture-based assays can be used to evaluate the ability of compounds of the invention to inhibit one or more cellular activities, such as cancer cell growth and/or survival. Various cancer cell lines can be obtained from the American Type Culture Collection (ATCC) and other sources. Briefly, cells are seeded into 96-well, tissue-culture treated, opaque white plates (Thermo Electron, Vantaa, Finland), at 1000 cells per well in 100 μL of appropriate growth medium (determined by the ATCC). Cells are then exposed to the appropriate concentration of drug and allowed to grow in its presence for 96 h. Following this, 100 μL of Cell-Titer-Glo reagent (Promega, Inc., Madison, Wis.) is added to each well. Plates are then shaken for 2 min at rt to allow for cell lysis and incubated for 10 min at rt to stabilize the luminescent signal. Similar to the Kinase-Glo assay reagent from Promega, this reagent contains both luciferase enzyme and its substrate luciferin. Luciferase, activated by ATP in the cell lysate, catalyzes the conversion of luciferin to oxyluciferin, a reaction which produces light. The amount of light produced is proportionate to the amount of ATP in the cell lysate, which is itself proportional to cell number and gives an index of cellular proliferation.

Two EXAMPLE compounds displayed IC₅₀ results with the above assay of about 0.448 μM and about 0.266 μM. It is advantageous that the IC₅₀ be less than 5.0 μM. It is more advantageous that the IC₅₀ be less than 1.0 μM.

Human Phospho-Axl ELISA Assay (R&D Systems Minneapolis, Minn.)

This assay utilizes a capture antibody specific for human Axl kinase bound to a 96-well plate. This antibody will recognize both phosphorylated and non-phosphorylated Axl. Cell lysate are incubated with the capture antibody, then washed to remove unbound proteins. An HRP-conjugated detection antibody specific for phosphorylated tyrosine residues is incubated in the assay wells to detect phospho-Axl kinase. A substrate solution is added followed by an acidic stop solution creating a color change according to the quantity of bound HRP detection antibody. The optical density of the colorometric change is determined using a microplate reader which reads at 450 nm and 540 nm. The reading at 540 nm is subtracted from the reading at 450 nm to correct for optical imperfections in the plate.

Cells plated at 4×10⁵ cells/mL in 6-well plates and incubated overnight are treated with Axl inhibitors and after a 4 h incubation are stimulated with 500 ng/mL GAS6 (Growth Arrest Specific gene 6) for 5 min. Cells are lysed by scraping in lysis buffer which consists of 1× R&D Systems IC Diluent #12 (Cat# DYC002, R&D Systems Minneapolis, Minn.) plus 1× protease inhibitor cocktail 111 (Cat# 80053-852 VWR International San Diego, Calif.). The protein is quantified using a BCA assay (Thermo Scientific, Rockford, Ill.). Capture antibody is added to each well of a 96-well ELISA ultra-high binding plate at a concentration of 8 μg/mL in PBS and incubated overnight at rt. Next day the plate is washed five times with wash buffer consisting of 0.05% Tween 20® in PBS, and then blocking the wells of the plate in 300 μL of 1% BSA and 0.05% sodium azide in PBS for 2 h at rt. The wells are washed five times following blocking and 125 μg of protein in 1× IC Diluent #12 is added to each well in 1000, volume. The protein lysates are incubated with the wells with bound capture antibody for 2 h at rt. The wells are washed five times with wash buffer and then incubated with 1004, of 1/1300 dilution of detection antibody in IC Diluent #14 consisting of 20 mM Tris, 137 mM NaCl, 0.05% Tween 20, 0.1% BSA in water. This incubation is for 2 h at rt, and is followed by washing five times with wash buffer. A substrate solution is mixed in a 1:1 ratio of reagent A and reagent B (Cat# DY999 R&D Systems Minneapolis, Minn.) then 100 μL/well are added and incubated for 20 min at rt. Following this incubation 50 μL of stop solution (Cat# DY994 R&D Systems Minneapolis, Minn.) is added directly to the substrate solution, mixed by tapping, and immediately measured for optical density.

Analysis of the data is done in Excel after manual subtraction of the 540 nm reading from the 450 nm reading. The mean relative fold change in phospho-Axl expression is calculated by averaging the optical densities of the no treatment samples and then dividing each sample optical density reading by the mean optical density reading of the no treatment to obtain the relative phospho-Axl values. The triplicate technical replicates' relative phospho-Axl values are averaged to calculate the mean relative phospho-Axl values for each sample and the standard error for each sample. Bar graphs are produced relative to the GAS6 treated samples and the percent activity or the effective concentration at 50% (EC₅₀) is calculated.

In one example of this assay, MDA-MB 231 cells were used, which are high in Axl expression. The mean relative fold change was plotted against the concentration of an EXAMPLE of the invention. The GAS6 treated cells (without dosing by the EXAMPLE) was set as 100% pAxl and no GAS6 treatment (also without dosing by the EXAMPLE) as zero baseline. The EXAMPLE showed EC₅₀ of about 0.8 μM.

Luminex® Phospho-AKT (S473) Assay

The Luminex® (Luminex® Corporation, Austin, Tex.) assay platform is a system which allows for multiple proteins in their native conformational state to be analyzed for expression directly from living systems. The components of the system simply consist of the target analyte of interest—such as a phosphorylated protein—polystyrene microspheres, instrument fluidics, instrument optics, and high speed data processing. The carboxylated polystyrene beads contribute to the flexibility of the assay platform in that various analyte capture species can be covalently attached to the surface of the microspheres. In addition, each microsphere in a set of 100 different beads is filled with a gradient mixture of red/infrared dyes, thus giving each bead its own signature dye mix. This individual dye mix within each bead enables the Luminex® instrument to identify which bead is passing through the optics system, and, given that each bead within a set of 100 beads can have a different capture analyte species bound to its surface, up to 100 different analytes can be assayed for in each well of a 96-well plate. Thus, anywhere from one analyte in 96 different samples up to 100 analytes in 96 different samples can be observed with this platform. The Bio-Plex™ Phosphoprotein Detection Kit (Bio-Rad Laboratories Inc., Hercules, Calif.) is a specific application of the platform for the phospho-AKT (S473) assay. In this case, only one analyte was analyzed. The phospho-AKT (S473) single plex was done with MDA-MB 231 and U2-OS cells as described here:

The Bio-Plex™ (Bio-Rad Laboratories Inc., Hercules, Calif.) phospho-AKT (S473) kit is done over a three day period. The evening of the first day, cells in full media are plated in a clear flat-bottom black-walled 96-well plate (Greiner Bio-One North America Inc., Monroe, N.C.) at a density of 35,000 cells/well for MDA-MB 231 (American Type Culture Collection, Manassas, Va.) cells and 30,000 cells/well for U2-OS (American Type Culture Collection, Manassas, Va.) cells. The next morning, the cells are treated with Axl inhibitors at a half-log concentration range of 3 μM to 0.03 μM with incubation for 10 min at 37° C. and 5% CO₂. After 10 min, 2.5 μg/mL of recombinant human GAS6 (rhGAS6) (R&D Systems Inc., Minneapolis, Minn.) is added to all but the no treatment wells for a 5 min incubation at 37° C. and 5% CO₂. The media is removed after the incubation, and the cells are washed with ice-cold PBS (HyClone Laboratories Inc., Logan, Utah). PBS is removed and the cells are lysed with lysis buffer provided in the Bio-Plex™ kit. The cells in the plate are scraped with pipette tips then placed on a plate shaker in 4° C. for 20 min with shaking at 600 rpm. Following shaking, the cell lysates are transferred to a clear v-bottom 96-well plate (Greiner Bio-One North America Inc., Monroe, N.C.) and centrifuged at 4,500 rcf for 20 min at 4° C. The cell lysates are stored on ice while the filter plate and the Bio-Plex™ beads included in the kit are prepared via washing steps with washing buffer included in the kit. Once the filter plate and the Bio-Plex™ beads, specific for phospho-AKT (S473) are prepared, 50 μL/well of cell lysate are added, which, given the cell plating density, equals 400 μg/well of protein. The filter plate containing the Bio-Plex™ beads in the protein lysates are placed in a covered shaker overnight at rt to shake at 600 rpm. The following morning the Luminex® instrument is prepared while the Bio-Plex™ beads are washed three times with wash buffer, and then incubated for 30 min with a secondary detection antibody specific for phospho-AKT (S473). Following the incubation, the Bio-Plex™ beads in the filter plate are washed three more times and incubated with a streptavidin-phycoerythrin (SAPE) stain from the kit that allows the optics of the Luminex® system to detect which beads have the target analyte of phospho-AKT(S473) bound to them. The beads in the SAPE solution are incubated in the covered shaker for 10 min at rt then rinsed three times with a rinse buffer from the Bio-Plex™ kit. Following the rinse, the plate is shaken briefly at 1,100 rpm to resuspend the beads. The plate is then placed in the tray of the Luminex® instrument and the samples analyzed.

Once the analysis of the plate has taken place, the raw numbers generated by the read out of mean fluorescence intensity (MFI) are utilized to calculate the mean relative percent phosphorylation of phospho-AKT (S473). The relative percent phosphorylation was calculated from the rhGAS6 only group by dividing the background-subtracted MFI values of each sample by the mean background-subtracted MFI values of the rhGAS6 only samples. This gives the relative percent phosphorylation of each sample technical replicate which are then averaged together to produce the mean relative percent phosphorylation for each replicate sample. This data analysis allows for comparisons between the no treatment group and the rhGAS6 treated cells to verify agonist responses of the cells to rhGAS6, as well as comparisons between the Axl inhibitor treated cells and the rhGAS6 only treated cells to verify efficacy of the Axl inhibitors in blocking Axl/GAS6 signal transduction pathways which are propagated by serine 473 activated AKT. An EC₅₀ can be calculated to determine the concentration at which the phosphorylation of Axl is inhibited by 50% with the equation of a line formula. Thus, compounds can be compared to each other for potency of Axl inhibition.

The tested EXAMPLES of the present invention displayed EC₅₀ results in the above Bio-Plex™ assay ranging from about 1.06 μM to about 0.455 μM. It is advantageous that the EC₅₀ results be less than 1.0 μM. It is more advantageous that the IC₅₀ results be less than 0.5 μM.

Phospho-Axl Immunoprecipitation Assay

Axl, being a receptor tyrosine kinase, is activated by ligand binding from GAS6 via phosphorylation events. There is currently no known specific phospho-tyrosine residue on Axl Kinase that is phosphorylated upon ligand binding, and therefore, there are no commercially available antibodies to probe for phosphorylated Axl. As such, the technique of immunoprecipitation can be employed to exploit the ability to observe phospho-Axl. The concept of immunoprecipitation is simple. A DNA plasmid encoding Axl Kinase along with a small protein tag, such as FLAG®, is transiently transfected into cells. The cells are allowed to grow and overexpress the tagged Axl protein. Upon cell lysis, the soluble Axl protein contained in the cellular cytoplasm is mixed with a buffer and agarose beads conjugated to antibodies specific for the FLAG® protein tag. In this way, only the overexpressed Axl protein tagged with FLAG® is bound to the antibody-bead system. By low speed centrifugation, the agarose beads now bound with FLAG® tagged Axl are spun down out of the supernatant and isolated from the nonessential proteins. A series of wash steps ensures that only the Axl protein is bound to the agarose, which can then be released from the antibody-bead system by protein reduction and denaturation. Once the protein has been denatured and reduced to become linear, it can be separated via polyacrylamide gel electrophoresis. The protein contained in the gel can be transferred to a nitrocellulose membrane for Western Blot detection of phospho-Axl by using a general phospho-tyrosine antibody. Likewise, total Axl protein can be detected by probing the membrane with an antibody which recognizes the FLAG® tagged Axl protein. Thus, detectable differences can be determined between total Axl levels and phosphorylated Axl levels from the transfected cells stimulated with GAS6 ligand.

The methods listed below were utilized with Immunoprecipitation techniques to obtain the data herein.

Cultured HEK 293 cells (American Type Culture Collection, Manassas, Va.) were counted to obtain 30×10⁶ cells/mL. The appropriate volume of media containing this cell count is centrifuged at 200 rcf for 10 min. The media was aspirated and the cell pellet resuspended in a sufficient volume of Cell Line Nucelofector® Solution V (Lonza Group Ltd., Basel, Switzerland) so as to yield 1004 per well of cells to be plated in the assay. This methodology provided for 3×10⁶ cells/well of a 6-well plate. After re-suspension the cells were aliquoted into 1.5 mL microcentrifuge tubes (USA Scientific Inc., Ocala, Fla.) with 100 μL/tube. To that suspension, 2 μg of Axl plasmid (Origene Technologies Inc., Rockville, Md.) were added, mixed and then placed into the electroporation cuvettes provided in the Amaxa® Cell Line Nucelofector® Kit V (Lonza Group Ltd., Basel, Switzerland). The cells were electroporated and then 500 μl of full media were added to the cuvette. The cell suspension was removed and placed into the 6-well plate (Becton Dickinson and Company, San Jose, Calif.) already containing 1.5 mL of pre-warmed full media. This process was repeated for each sample well after which the cells were incubated for 24 h at 37° C. and 5% CO₂. The following day, the cells were treated with Axl inhibitors at half-log concentrations from 3 μM to 0.01 μM for a 10 min incubation at 37° C. and 5% CO₂. After the 10 min incubation the cell culture media was removed and fresh, warm conditioned media from WI38 (American Type Culture Collection, Manassas, Va.) cells, which contained GAS6, was added. In addition, fresh Axl inhibitors were once again added at half-log concentrations from 3 μM to 0.01 μM and then incubated for 5 min at 37° C. and 5% CO₂. Upon completion of the 5 min incubation, the media was removed and the cells washed with ice-cold PBS (HyClone Laboratories Inc., Logan, Utah). The PBS was removed and the cells lysed with 100 mL of general cell lysis buffer—1% NP40, 120 mM NaCl, 30 mM Tris pH7.4, 1× protease inhibitors (EMD Chemical Inc., Darmstadt, Germany), 1× phosphatase inhibitors (Sigma-Aldrich Inc, St. Louis, Mo.). The cells were scrapped and the suspension pipetted into 1.5 mL microcentrifuge tubes. The tubes were incubated on ice for 10 min after which the lysates were centrifuged at 13,500 rpm for 15 s to clear the lysates. The supernatants were transferred to new microcentrifuge tubes and the protein quantified via a BCA kit (Thermo Fisher Scientific Inc., Rockford, Ill.). In separate microcentrifuge tubes 40 μL of Anti-FLAG® M2 Agarose (Sigma-Aldrich Inc, St. Louis, Mo.) were added and washed using TBS three times and kept at 4° C. After washing, 8004, of Rotation Buffer—TBS, 0.2% BSA (Sigma-Aldrich Inc, St. Louis, Mo.), 1× protease inhibitors (Promega Corporation Madison, Wis.), 2× phosphatase inhibitors (Sigma-Aldrich Inc, St. Louis, Mo.)—were added to the Anti-FLAG® M2 Agarose along with the appropriate volume of cell lysates to equal 20014 of protein/sample. The samples were rotated overnight in the 4° C. The next day, the samples were centrifuged at 250 rcf for 30 s at 4° C. and the supernatant removed. The Anti-FLAG® M2 Agarose in each tube was washed twice with Wash Buffer—TBS, and 0.1% BSA—then washed two more times with 15 min rotations at 4° C. for each wash. A final wash with TBS was done and then the samples with the Anti-FLAG® M2 Agarose were cooked in 70 μL of 2×LDS Buffer (Invitrogen Corporation Carlsbad, Calif.), 2× Sample Reducing Buffer (Invitrogen Corporation Carlsbad, Calif.) and water for 10 min at 70° C. After cooking, each sample is electrophoresed at 150 v for ˜1.5 h through a 4-12% Bis-Tris polyacrylamide gel (Invitrogen Corporation Carlsbad, Calif.) then transferred to a nitrocellulose membrane. Following transfer, the membrane is blocked using 5% non-fat dry milk solution in TBST—TBS solution with 0.05% Tween 20 (EMD Chemical Inc., Darmstadt, Germany)—for 1 h at rt. The primary antibody for phospho-Axl is an anti-phospho tyrosine PY20-HRP conjugate (Santa Cruz Biotechnology Inc., Santa Cruz, Calif.), which is used at 1:500 in 5% non-fat dry milk solution in TBST for 1 h at rt. The primary antibody for the total Axl is the anti-DDK antibody (Origene Technologies Inc., Rockville, Md.), which detects the FLAG epitope. It is used at 1:1000 in 5% non-fat dry milk solution in TBST for 1 h at rt. The membranes are washed after incubation with TBST three times for 5 min each. The PY20-HRP antibody for phospho-Axl is then developed by adding 1 mL of SuperSignal West Dura ECL (Thermo Fisher Scientific Inc., Rockford, Ill.) over the membrane and imaging with the Kodak In Vivo FX imager (Eastman Kodak Company, Rochester, N.Y.). The total Axl membrane is incubated with the secondary antibody, which is a goat anti-mouse-HRP (R&D Systems Inc., Minneapolis, Minn.) used at 1:1000 for 1 h at rt in 20% goat serum (Sigma-Aldrich Inc, St. Louis, Mo.) in TBST. The membrane is washed with TBST three times for 15 min each then developed in like manner.

The percent phosphorylation of the blots for phospho-Axl is determined by utilizing the Kodak In Vivo FX software to draw regions of interest (ROI) around the phospho-Axl and total Axl bands. The ROI information provides the Net Intensity values for the bands which are then used to normalize the phospho-Axl to the total Axl signal by dividing the phospho-Axl net intensity of each band by the net intensity of the total Axl signal of the same sample. The percent phosphoryation is then calculated by dividing the normalized values of each sample by the normalized value of the GAS6 only sample. Thus, a comparison between the no treatment sample and the GAS6 stimulated sample can be made to verify agonist efficacy, while a comparison between the GAS6 only sample and the GAS6 plus Axl inhibitor samples demonstrates the efficacy of the inhibitors to block Axl Kinase phosphorylation upon GAS6 ligand binding and stimulation. An EC₅₀ can be calculated to determine the concentration at which the phosphorylation of Axl is inhibited by 50% with the equation of a line formula. Thus, compounds can be compared to each other for potency of Axl inhibition.

The tested EXAMPLES of the present invention displayed EC₅₀ results in the above immunoprecipitation assay ranging from about 0.100 μM to about 0.068 μM.

Chemistry

Compounds of the invention may be made by one of ordinary skill in the chemical arts using conventional synthetic procedures, as well as by the general reaction schemes and examples described below.

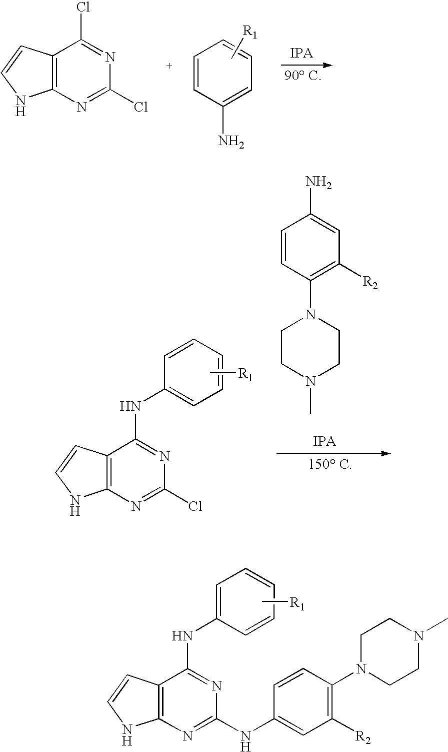

2-(3-(2-Chloro-7H-pyrrolo[2,3-d]pyrimidin-4-ylamino)phenyl)acetonitrile (COMPOUND A): The reaction mixture containing 2,4-dichloro-7H-pyrrolo[2,3-d]pyrimidine (500 mg, 2.66 mmol) and 2-(3-aminophenyl)acetonitrile (351 mg, 1 eq) was microwaved at 90° C. for 21 h. Concentration and combiflash (4 g, DCM to 10% MeOH/DCM) afforded a yellow powder. ¹HNMR (300 MHz, CD3OD) 7.82 (m, 2H), 7.37 (t, J=7.8 Hz, 1H), 7.13 (d, J=3.66 Hz, 1H), 7.10 (m, 1H), 6.69 (d, J=3.66 Hz, 1H), 3.93 (s, 2H) MS: 284.3 (M+H)+

2,2′-(3,3′-(7H-pyrrolo[2,3-d]pyrimidine-2,4-diyl)bis(azanediyl)bis(3,1-phenylene)) diacetonitrile (EXAMPLE 1): EXAMPLE 1 was isolated as side product during preparation of COMPOUND A. ¹H-NMR (300 MHz, DMSO-d6) 7.8 (m, 3H) 7.79 (s, 1H), 7.43 (m, 1H), 7.34 (m, 1H), 7.04 (m, 3H), 6.76 (s, 1H), 4.09 (s, 2H), 4.01 (s, 2H), MS: 380.31 (M+H)⁺

2-(3-(2-(3-fluoro-4-(4-methylpiperazin-1-yl)phenylamino)-7H-pyrrolo[2,3-d]pyrimidin-4-ylamino)phenyl)acetonitrile (EXAMPLE 2): To a reaction mixture of COMPOUND A (50 mg, 0.176 mmol) and 3-fluoro-4-(4-methylpiperazin-1-yl)aniline (36.9 mg, 1 eq) in IPA (5 mL) was added HCl (4M in Dioxane, 0.132 ml, 3 eq). The mixture microwaved at 150° C. for 1 h. HPLC (280 nm) indicated 33% conversion. Na₂CO₃ was added. Concentration and combiflash (4 g, DCM to 10% MeOH/DCM) gave a brown solid. ¹H NMR (300 MHz, CD₃OD) 7.98 (s, 1H), 7.81 (d, J=2.44 Hz, 1H), 7.75 (m, 1H), 7.32 (t, J=8.0 Hz, 1H), 76.19 (m, 1H), 7.00 (d, J=7.53 Hz, 1H), 6.87 (d, J=3.67 Hz, 1H), 6.59 (d, J=3.67 Hz, 1H), 6.43 (m, 1H), 3.87 (s, 2H), 3.04 (br-s, 4H), 2.62 (br-s, 4H), 2.34 (s, 3H). ¹⁹FNMR (300 Hz, CD₃OD) −188.92 MS: 457.5 (M+H)⁺

2-(3-(2-(4-(4-methylpiperazin-1-yl)phenylamino)-7H-pyrrolo[2,3-d]pyrimidin-4-ylamino)phenyl)acetonitrile (EXAMPLE 3): The procedure is similar to the preparation of EXAMPLE 2. ¹HNMR (300 MHz, CD₃OD) 7.95 (d, 1H), 7.71 (m, 1H), 7.56 (m, 2H), 7.32 (m, 1H), 7.15 (d, J=3.67 Hz, 1H), 6.97 (m, 2H), 6.83 (m, 1H), 6.60 (m, 1H), 3.81 (s, 1H), 3.63 (s, 1H), 3.15 (s, 4H), 2.63 (s, 4H), 2.35 (s, 3H). MS: 439.6 (M+H)⁺

2-(3-(2-(4-(4-methylpiperazin-1-yl)-3-(trifluoromethyl)phenylamino)-7H-pyrrolo[2,3-d]pyrimidin-4-ylamino)phenyl)acetonitrile (EXAMPLE 4): ¹HNMR (300 MHz, CD₃OD) 7.99 (m, 2H), 7.76 (dd, J1=1.23 Hz, J2=8.79 Hz, 1H), 7.27 (m, 2H), 7.15 (d, J=3.66 Hz, 1H), 7.02 (m, 1H), 6.88 (m, 1H), 6.62 (m, 1H), 3.96 (s, 1H), 3.87 (s, 1H), 2.89 (m, 4H), 2.64 (br-s, 4H), 2.37 (m, 3H), ¹⁹FNMR (300 MHz, CD3OD) −143.58, −143.8, −144.22 MS− ESI: 507.4 (M+H)⁺

2-(3-(2-(4-(4-cyclohexylpiperazine-1-carbonyl)phenylamino)-7H-pyrrolo[2,3-d]pyrimidin-4-ylamino)phenyl)acetonitrile (EXAMPLE 5): ¹H-NMR (300 MHz, CD₃OD) 8.08 (d, J=9.52 Hz, 1H), 7.8 (m, 1H), 7.62 (d, J=7.81 Hz, 1H), 7.52 (d, J=7.81 Hz, 1H), 7.41 (t, J=8.05 Hz, 1H), 7.28 (m, 1H), 7.15 (d, J=6.6 Hz, 1H), 7.09 (d, J=3.66 Hz, 1H), 6.81 (d, J=3.42 Hz, 1H), 6.54 (m, 1H), 3.93 (s, 1H), 3.85 (s, 1H), 3.78 (s, 4H), 2.70 (m, 4H), 1.89 (m, 2H), 1.76 (m, 2H), 1.20 (m, 6). MS: 535.4 (M+H)⁺

2-(3-(2-(4-(4-methylpiperazine-1-carbonyl)phenylamino)-7H-pyrrolo[2,3-d]pyrimidin-4-ylamino)phenyl)acetonitrile (EXAMPLE 6): ¹H-NMR (300 MHz, CD₃OD) 8.06 (s, 1H), 7.73 (d, J=7.33 Hz, 1H), 7.63 (s, 1H), 7.42 (d, J=7.81 Hz, 1H), 7.28 (m, 1H), 7.18 (m, 1H), 7.11 (m, 1H), 6.92 (m, 1H), 6.82 (m, 1H), 6.55 (d, J=3.42 Hz, 1H), MS: 467.4 (M+H)⁺

2-(3-(2-(4-(4-cyclohexylpiperazin-1-yl)phenylamino)-7H-pyrrolo[2,3-d]pyrimidin-4-ylamino)phenyl)acetonitrile (EXAMPLE 7): ¹HNMR (300 MHz, CD₃OD) 7.92 (s, 1H), 7.72 (m, 1H), 7.55 (d, J=9.04 Hz, 1H), 7.47 (d, J=8.5 Hz, 1H), 7.29 (m, 1H), 7.14 (d, J=3.66 Hz, 1H), 6.88 (m, 2H), 6.60 (d, J=3.9 Hz, 1H), 3.93 (s, 1H), 3.77 (s, 0.5H), 3.63 (s, 0.5H), 3.08 (s, 4H), 2.75 (s, 4H), 2.32 (br-s, 1H), 1.91 (s, 2H), 1.79 (s, 2H), 1.97 (m, 4H). MS: 507.5 (M+H)⁺

2-(4-(2-chloro-7H-pyrrolo[2,3-d]pyrimidin-4-ylamino)phenyl)acetonitrile (COMPOUND B): 2,4-Dichloro-7H-pyrrolo[2,3-d]pyrimidine (500 mg) and 2-(4-aminophenyl)acetonitrile (351 mg) was mixed in 1-butanol and heated at 90° C. for 24 h. Yellow precipitation formed from clear solution. Concentration and combiflash (12 g, DCM to 10% MeOH/DCM) afforded a brown solid. ¹H NMR (300 MHz, CD₃OD) 7.83 (d, J=8.44 Hz, 2H), 7.35 (d, J=8.44 Hz, 2H), 7.13 (d, J=3.66 Hz, 1H), 6.68 (d, J=3.66 Hz, 1H), 3.88 (s, 2H) MS-ESI (NEG): 282.1, 284.1 (M−H)⁻

2-(4-(2-(3-fluoro-4-(4-methylpiperazin-1-yl)phenylamino)-7H-pyrrolo[2,3-d]pyrimidin-4-ylamino)phenyl)acetonitrile (EXAMPLE 8): The procedure is similar to the preparation of EXAMPLE 2. ¹HNMR (300 MHz, CD₃OD) 7.86 (m, 2H), 7.50 (d, J=8.55 Hz, 1H), 7.30 (d, J=8.55 Hz, 1H), 7.16 (m, 2H), 6.90 (m, 1H), 6.86 (d, J=3.66 Hz, 1H), 6.58 (m, 1H), 3.97 (s, 1H), 3.86 (s, 1H), 3.05 (br-s, 4H), 2.63 (br-s, 4H), 2.34 (s, 3H). ¹⁹F NMR ((300 MHz, CD₃OD) −206.38, MS: 457.4 (M+H)⁺

N-(3-(2-chloro-7H-pyrrolo[2,3-d]pyrimidin-4-ylamino)phenyl)cyclopropane carboxamide (COMPOUND C): The procedure is similar to the preparation of COMPOUND B. ¹H NMR (300 MHz, CD₃OD) 7.94 (s, 1H), 7.51 (d, J=7.32 Hz, 1H), 7.305 (m, 2H), 7.10 (d, J=3.66 Hz, 1H), 6.63 (d, J=3.66 Hz, 1H), 1.78 (m, 1H), 0.95 (m, 2H), 0.85 (m, 2H). MS: (NEG) 326.3 (M−H)⁻

N-(3-(2-(3-fluoro-4-(4-methylpiperazin-1-yl)phenylamino)-7H-pyrrolo[2,3-d]pyrimidin-4-ylamino)phenyl)cyclopropanecarboxamide (EXAMPLE 9): ¹H NMR (300 MHz, CD₃OD) 8.14 (s, 1H), 7.90 (dd, J1=2.44 Hz, J2=5.63 Hz, 1H), 7.45 (m, 2H), 7.17 (m, 2H), 6.86 (m, 2H), 6.60 (d, J=3.66 Hz, 1H), 3.03 (s, 4H), 2.61 (s, 4H), 2.33 (s, 3H), 1.8 (m, 1H), 0.95 (m, 2H), 0.85 (m, 2H), ¹⁹F-NMR (300 MHz, CD₃OD) −205.86 MS: 501.4 (M+H)⁺

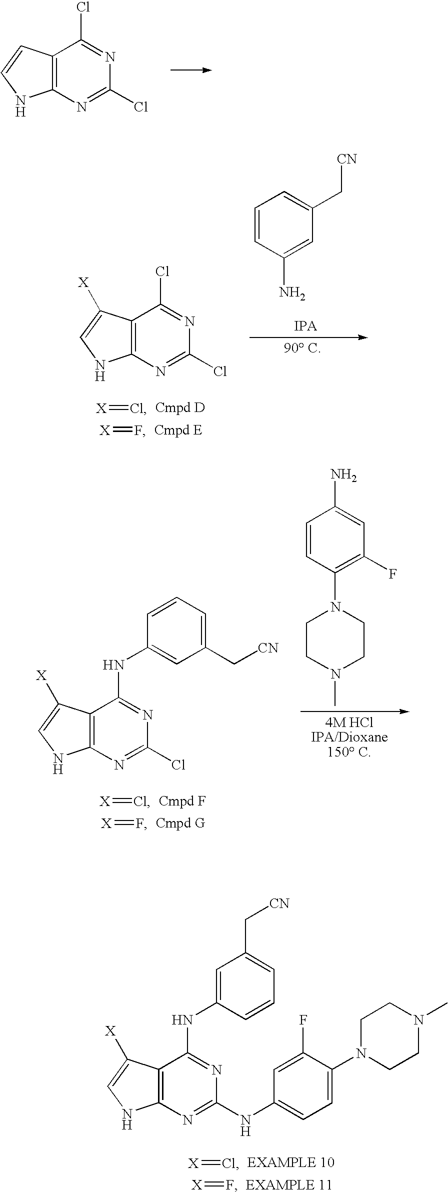

2,4,5-trichloro-7H-pyrrolo[2,3-d]pyrimidine (COMPOUND D): The reaction mixture of 2,4-Dichloro-7H-pyrrolo[2,3-d]pyrimidine (0.1 g, 0.532 mmol) and NCS (0.132 g, 1.2 eq) in DCM/THF (10 mL/4 mL) was heated by microwave at 90° C. for 2.5 h. Concentration and combiflash (10 g, DCM) afforded a white solid. ¹H-NMR (300 MHz, CDCl₃) 10.34 (br-s, 1H), 7.35 (s, 1H), GC-MS: 221, 223 (M⁺)

2,4-dichloro-5-fluoro-7H-pyrrolo[2,3-d]pyrimidine (COMPOUND E): To a solution of ,4-Dichloro-7H-pyrrolo[2,3-d]pyrimidine 90.3 g, 1.6 mmol), Selectfluor (0.848 g, 2.4 mmol) was added acetonitrile (15 mL) and AcOH (2.5 mL). The solution was then heated at 70° C. for 24 h under Ar. After cooling to rt, ice was added and the mixture was neutralized by NaHCO₃. Extraction with EtOAc and the organic residue was purified by combiflash (10 g, 0%˜100% EtOAc/Hexane). White crystal was obtained. ¹HNMR (300 MHz, (CD₃)₂CO) 7.60 (d, J=3.6 Hz, 1H). MS-ESI: 206.1, 208.1 (M+H)⁺

2-(3-(2,5-dichloro-7H-pyrrolo[2,3-d]pyrimidin-4-ylamino)phenyl)acetonitrile (COMPOUND F): The procedure is similar to the preparation of EXAMPLE 2. ¹HNMR (300 MHz, (CD₃)₂CO) 8.29 (s, 1H), 7.89 (m, 2H), 7.45 (m, 2H), 7.20 (d, J=7.33 Hz, 1H) 4.03 (s, 2H), MS: 318.2, 320.2 (M+H)⁺

2-(3-(2-chloro-5-fluoro-7H-pyrrolo[2,3-d]pyrimidin-4-ylamino)phenyl)acetonitrile (COMPOUND G): ¹HNMR (300 MHz, (CD3)₂CO) 10.8 (br-s, 1H), 8.54 (s, 1H), 7.90 (m, 2H), 7.42 (m, 1H), 7.19 (m, 2H), 4.03 (s, 2H), ¹⁹FNMR (300 MHz, (CD₃)₂CO) −249.74 (s) MS-ESI: 302.2, 304.2 (M+H)⁺,

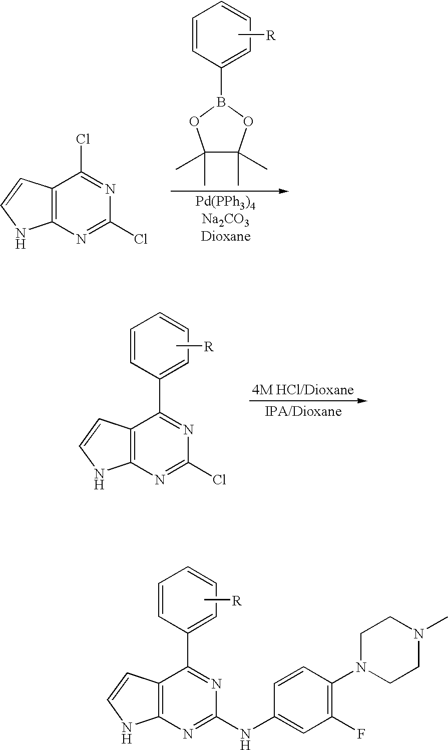

2-(3-(2-chloro-7H-pyrrolo[2,3-d]pyrimidin-4-yl)phenyl)acetonitrile (COMPOUND H): To the mixture of 2,4-dichloro-7H-pyrrolo[2,3-d]pyrimidine (0.5 g, 2.66 mmol), 2-(3-(4,4,5,5-tetramethyl-1,3,2-dioxaborolan-2-yl)phenyl)acetonitrile (647 mg, 1 eq) and Pd(PPh₃)₄ (92 mg, 0.08 mmol), was added Na₂CO₃ (2M, 3.99 mL, 3 eq) and dioxane (16 mL). The reaction mixture was heated at 150° C. for 1 h by microwave. Concentration and combiflash (40 g, DCM to 10% MeOH/DCM) afforded a yellow powder. ¹HNMR (300 MHz, CDCl₃) 8.10 (m, 1H), 7.55 (m, 3H), 7.18 (m, 1H), 6.86 (s, 1H), 3.88 (s, 2H). MS: 269.2 (M+H)⁺

2-(3-(2-(3-fluoro-4-(4-methylpiperazin-1-yl)phenylamino)-7H-pyrrolo[2,3-d]pyrimidin-4-yl)phenyl)acetonitrile (EXAMPLE 12): The reaction condition is similar to the preparation of EXAMPLE 2. The product was purified with silica gel combiflash and C-18 RediSep column to remove all starting materials. ¹HNMR (300 Mhz, CD₃OD) 8.16 (s, 1H), 8.09 (d, J=7.32 Hz, 1H), 7.90 (d, J=15.14 Hz, 1H), 7.54 (m, 2H), 7.36 (d, J=8.54 Hz, 1H), 7.17 (m, 1H), 7.01 (t, J=9.04 Hz, 1H), 6.68 (m, 1H), 6.43 (m, 1H), 4.033 (s, 2H), 3.09 (s, 4H), 2.75 (s, 4H), 2.43 (s, 3H). ¹⁹FNMR (300 MHz, CD₃OD) −206,32 (m) MS: 442.4 (M+H)⁺

2-chloro-4-(3-(methoxymethyl)phenyl)-7H-pyrrolo[2,3-d]pyrimidine (COMPOUND H): ¹HNMR (300 MHz, CDCl₃) 8.15 (s, 1H), 8.09 (d, J=6.11 Hz, 1H), 7.57 (m, 2H), 7.45 (s, 1H), 6.91 (s, 2H), 4.62 (s, 1H), 3.47 (s, 1H), MS-ESI: 274.1 (M+H)⁺

N-(3-fluoro-4-(4-methylpiperazin-1-yl)phenyl)-4-(3-(methoxymethyl)phenyl)-7H-pyrrolo[2,3-d]pyrimidin-2-amine (EXAMPLE 13): ¹HNMR (300 MHz, CD₃OD): 8.10 (s, 1H), 8.03 (d, J=7.57 Hz, 1H), 7.93 (dd, J₁=2.44 Hz, J₂=5.38 Hz, 1H), 7.50 (m, 2H), 7.28 (m, 1H), 7.14 (d, J=3.67 Hz, 1H), 6.95 (m, 1H), 6.66 (d, J=3.66 Hz, 1H), 6.42 (m, 1H), 4.55 (s, 2H), 3.35 (s, 3H), 3.04 (s, 4H), 2.61 (s, 3H), 2.33 (s, 3H). ¹⁹FNMR (300 MHz, CD₃OD): −206.26 MS-ESI: 447.4 (M+H)⁺

2-chloro-4-(3-(trifluoromethoxy)phenyl)-7H-pyrrolo[2,3-d]pyrimidine (COMPOUND J): ¹HNMR (300 MHz, (CD₃)₂CO) 8.30 (d, J=7.81 Hz, 1H), 8.16 (s, 1H), 7.79 (m, 2H), 7.60 (d, J=8.05 Hz, 1H), 7.04 (d, J=3.67 Hz, 1H), MS-ESI (NEG): 312.2 (M−H)⁻

3-(2-chloro-7H-pyrrolo[2,3-d]pyrimidin-4-yl)-N,N-dimethylaniline (COMPOUND K): ¹HNMR (300 MHz, (CD₃)₂CO) 7.67 (d, J=3.67 Hz, 1H), 7.50 (m, 3H), 6.99 (m, 2H), 3.08 (s, 6H) MS-ESI (NEG): 271.1, 273.3 (M−H)⁻

N-(3-fluoro-4-(4-methylpiperazin-1-yl)phenyl)-4-(3-(trifluoromethoxy)phenyl)-7H-pyrrolo[2,3-d]pyrimidin-2-amine (EXAMPLE 14): ¹HNMR (300 MHz, CD₃OD): 8.15 (d, J=7.81 Hz, 1H), 8.06 (s, 1H), 7.95 (dd, J₁=2.2 Hz, J₂=5.63 Hz, 1H), 7.66 (t, J=8.05 Hz, 1H), 7.43 (d, J=7.56 Hz, 1H), 7.33 (d, J=8.79 Hz, 1H), 7.20 (d, J=3.66 Hz, 1H), 6.99 (t, J=9.28 Hz, 1H), 6.66 (d, J=3.66 Hz, 1H), 3.08 (br-s, 4H), 2.65 (br-s, 4H), 2.36 (s, 3H). ¹⁹FNMR (300 MHz, CD₃OD): −141.496 (s), −206.32 (q) MS-ESI (NEG): 485.4 (M−H)⁻

4-(3-(dimethylamino)phenyl)-N-(3-fluoro-4-(4-methylpiperazin-1-yl)phenyl)-7H-pyrrolo[2,3-d]pyrimidin-2-amine (EXAMPLE 15): ¹HNMR (300 MHz, CD₃OD): 8.44 (dd, J₁=2.15 Hz, J₂=5.77 Hz, 1H), 8.16 (d, J=1.95 Hz, 1H), 7.71 (d, J=9.04 Hz, 1H), 7.52 (d, J=8.8 Hz, 1H), 7.34 (m, 2H), 7.14 (m, 2H), 7.00 (m, 2H), 3.63 (s, 3H), 3.14 (s, 3H), 3.08 (s, 4H), 2.64 (br-s, 4H), 2.35 (s, 3H). ¹⁹FNMR (300 MHz, CD₃OD): −206.22 MS-ESI: 446.5 (M+H)⁺

2-(4-(2-chloro-7H-pyrrolo[2,3-d]pyrimidin-4-yl)phenyl)acetonitrile (COMPOUND L): ¹HNMR (300 MHz, (CD₃)₂CO) 8.30 (d, J=8.3 Hz, 2H), 7.69 (m, 3H), 7.05 (d, J=3.66 Hz, 1H), 4.16 (s, 2H). MS-ESI (NEG): 267.0 (M−H)⁻

Representative EXAMPLES of the invention are set forth below in Tables 1 and 2 below:

TABLE 1

| EX. | Structure |
|---|---|
| 1 | 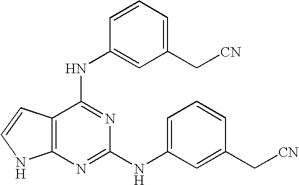 |
| 2 | 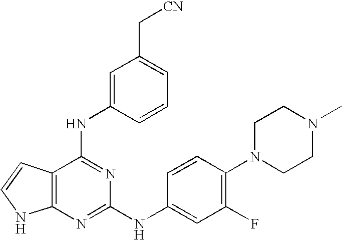 |
| 3 | 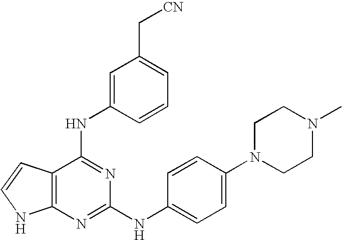 |
| 4 | 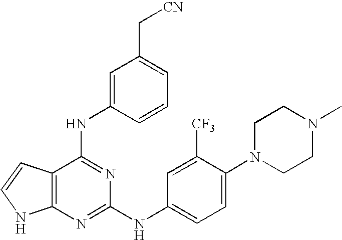 |
| 5 | 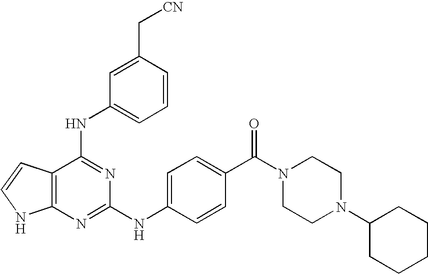 |
| 6 | 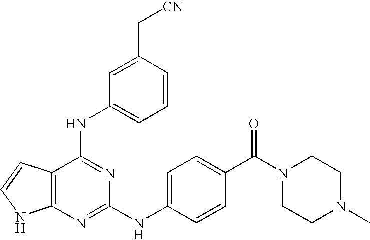 |
| 7 | 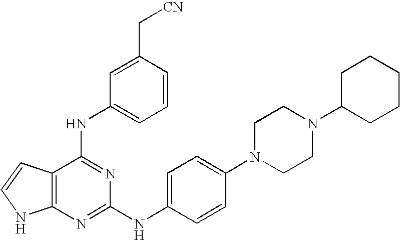 |
| 8 | 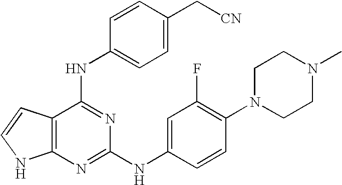 |
| 9 | 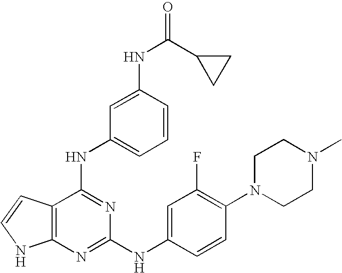 |
| 10 | 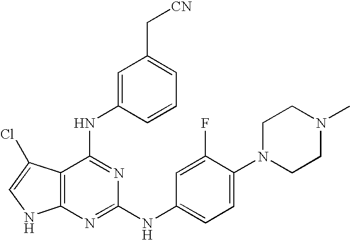 |
| 11 | 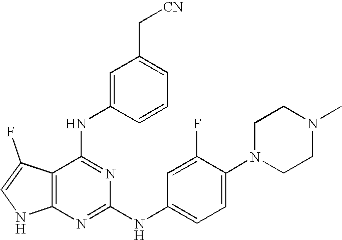 |
| 16 | 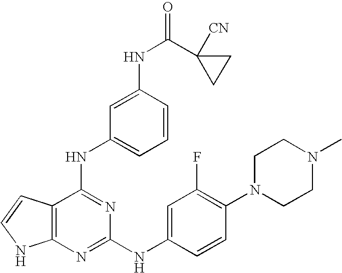 |

TABLE 2

| EX. | Structure |
|---|---|
| 12 | 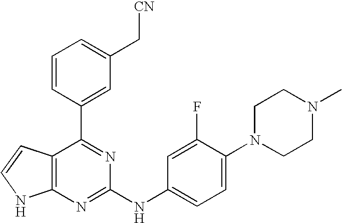 |
| 13 | 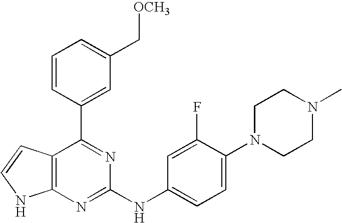 |
| 14 | 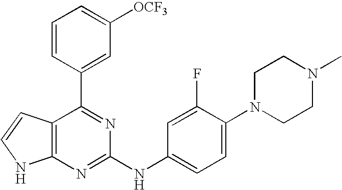 |
| 15 | 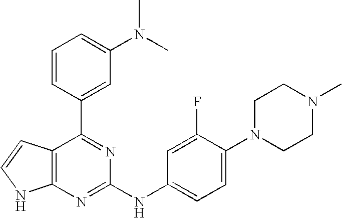 |

The compounds of the present invention include:
2,2′-(3,3′-(7H-pyrrolo[2,3-d]pyrimidine-2,4-diyl)bis(azanediyl)bis(3,1-phenylene))diacetonitrile;
2-(3-(2-(3-fluoro-4-(4-methylpiperazin-1-yl)phenylamino)-7H-pyrrolo[2,3-d]pyrimidin-4-ylamino)phenyl)acetonitrile;
2-(3-(2-(4-(4-methylpiperazin-1-yl)phenylamino)-7H-pyrrolo[2,3-d]pyrimidin-4-ylamino)phenyl)acetonitrile;
2-(3-(2-(4-(4-methylpiperazin-1-yl)-3-(trifluoromethyl)phenylamino)-7H-pyrrolo[2,3-d]pyrimidin-4-ylamino)phenyl)acetonitrile;
2-(3-(2-(4-(4-cyclohexylpiperazine-1-carbonyl)phenylamino)-7H-pyrrolo[2,3-d]pyrimidin-4-ylamino)phenyl)acetonitrile;
2-(3-(2-(4-(4-methylpiperazine-1-carbonyl)phenylamino)-7H-pyrrolo[2,3-d]pyrimidin-4-ylamino)phenyl)acetonitrile;
2-(3-(2-(4-(4-cyclohexylpiperazin-1-yl)phenylamino)-7H-pyrrolo[2,3-d]pyrimidin-4-ylamino)phenyl)acetonitrile;
2-(4-(2-(3-fluoro-4-(4-methylpiperazin-1-yl)phenylamino)-7H-pyrrolo[2,3-d]pyrimidin-4-ylamino)phenyl)acetonitrile;
N-(3-(2-(3-fluoro-4-(4-methylpiperazin-1-yl)phenylamino)-7H-pyrrolo[2,3-d]pyrimidin-4-ylamino)phenyl)cyclopropanecarboxamide;
2-(3-(2-(3-fluoro-4-(4-methylpiperazin-1-yl)phenylamino)-7H-pyrrolo[2,3-d]pyrimidin-4-yl)phenyl)acetonitrile;
N-(3-fluoro-4-(4-methylpiperazin-1-yl)phenyl)-4-(3-(methoxymethyl)phenyl)-7H-pyrrolo[2,3-d]pyrimidin-2-amine;
N-(3-fluoro-4-(4-methylpiperazin-1-yl)phenyl)-4-(3-(trifluoromethoxy)phenyl)-7H-pyrrolo[2,3-d]pyrimidin-2-amine;
4-(3-(dimethylamino)phenyl)-N-(3-fluoro-4-(4-methylpiperazin-1-yl)phenyl)-7H-pyrrolo[2,3-d]pyrimidin-2-amine;

and pharmaceutically acceptable salts thereof.

Other EXAMPLES of the present invention are shown in the following table:

| EX. | Structure |
|---|---|
| 17 | 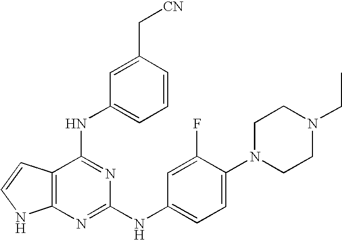 |
| 18 | 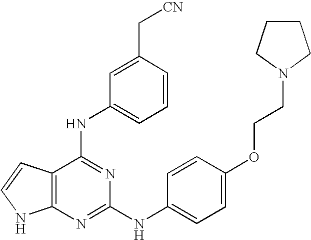 |
| 19 | 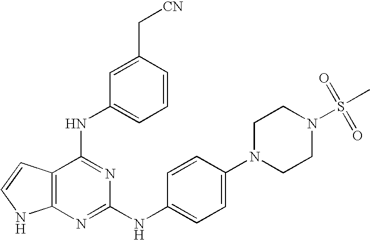 |
| 20 | 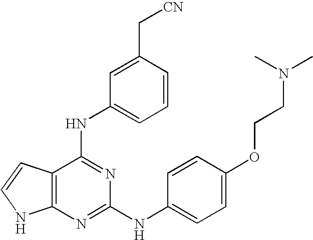 |
| 21 | 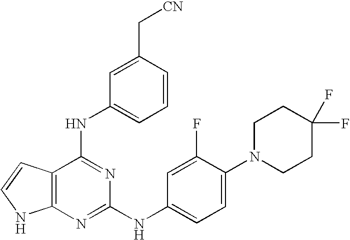 |
| 22 | 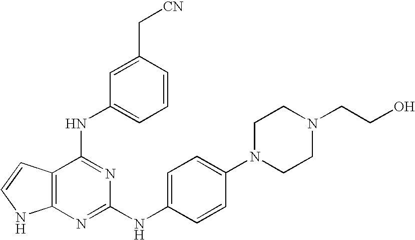 |
| 23 | 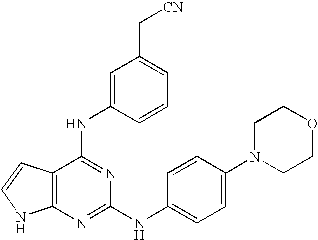 |

EXAMPLES 17-23 can be made using the following precursors and by the general synthetic routes described herein.

Synthesis of Precursors:

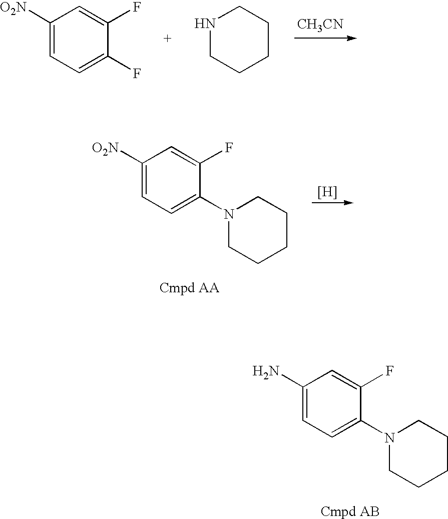

### 1-(2-Fluoro-4-nitrophenyl)piperidine (Cmpd AA)

To large round bottom flask was added acetonitrile (40 mL), 3,4-difluoronitrobenzene (0.278 mL, 2.51 mmol), and piperidine (0.298 mL, 3.02 mmol). Upon addition of the piperidine, the solution changed from a clear to a yellow transparent solution. The resulting solution was refluxed at 80° C. overnight (16 h) with stirring. TLC (30% DCM/Hexanes) showed reaction completion. CombiFlash (10 g, Hexanes to DCM) afforded Cmpd AA as yellow oil. ¹HNMR (300 MHz, CDCl₃): 7.98-7.97 (ddd, J₁=0.98 Hz, J₂=2.68 Hz, J₃=9.03 Hz, 1H), 7.91-7.85 (dd, J₁=2.68 Hz, J₂=13 Hz, 1H), 6.92-6.86 (dd, J₁=8.79 Hz, J₂=9.03 Hz, 1H), 3.28-3.24 (t, J=5 Hz, 4H), 1.77-1.62 (m, 6H) MS: 225.0 (M+H)+

### 3-Fluoro-4-(piperidin-1-yl)aniline (Cmpd AB)

To a solution of Cmpd AA (506 mg, 2.257 mmol) in ethanol (50 mL) was added a Pd/C (100 mg, 0.094 mmol) catalyst. The black suspension was shaken under hydrogen atmosphere (60 psi) for 2 h. TLC (DCM) showed reaction completion. CombiFlash (10 g, Hexanes to EtOAc) afforded Cmpd AB as a reddish-brown oil. ¹HNMR (300 MHz, CD₃OD): 6.90-6.84 (dd, J₁=J₂=8.3 Hz, 1H), 6.49-6.43 (m, 2H), 2.89-2.86 (t, J=5.2 Hz, 4H), 1.76-1.68 (m, 4H), 1.59-1.53 (m, 2H) ¹⁹FNMR (300 MHz, CD₃OD): −207.21 to −207.30 (m, 1F) MS: 195.1 (M+H)⁺

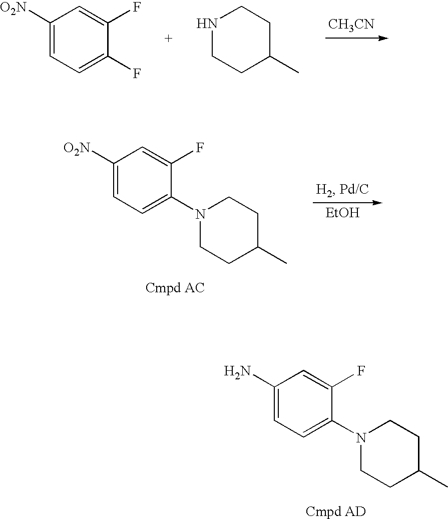

### 1-(2-fluoro-4-nitrophenyl)-4-methylpiperidine (Cmpd AC)

To a large round bottom flask was added acetonitrile (45 mL), 3,4-difluoronitrobenzene (0.313 mL, 2.83 mmol), and 4-methylpiperidine (0.418 mL, 3.39 mmol). The solution was refluxed (80° C.) with stirring overnight (16 h). TLC (30% DCM/Hexanes) showed reaction completion. CombiFlash (10 g, Hexanes to DCM) afforded Cmpd AC as a yellow oil. ¹HNMR (300 MHz, CDCl₃): 8.00-7.99 (ddd, J₁=0.97 Hz, J₂=2.68 Hz, J₃=9.03 Hz, 1H), 7.92-7.87 (dd, J₁=2.68 Hz, J₂=13.5 Hz, 1H), 7.11-7.05 (dd, J₁=J₂=9.03 Hz, 1H), 3.72-3.67 (m, 2H), 2.93-2.84 (m, 2H), 1.79-1.74 (m, 2H), 1.64-1.57 (m, 1H), 1.42-1.28 (m, 2H), 1.01-0.99 (d, J=6.6 Hz, 3H) ¹⁹FNMR (300 MHz, CDCl₃): −203.11 to −203.19 (dd, J₁=9.15 Hz, J₂=13.73 Hz) MS: 239.2 (M+H)⁺

### 3-Fluoro-4-(4-methylpiperidin-1-yl)aniline (Cmpd AD)

To a solution of Cmpd AC (559 mg, 2.346 mmol) in ethanol (55 mL) was added a Pd/C (112 mg, 0.105 mmol) catalyst. The resulting black suspension was shaken for 2 h under a hydrogen atmosphere (60 psi). TLC (6% MeOH/DCM) showed reaction completion. CombiFlash (10 g, Hexanes to EtOAc) afforded Cmpd AD as a solid. ¹HNMR (300 MHz, CDCl₃): 6.84-6.78 (dd, J₁=8.5 Hz, J₂=9.5 Hz, 1H), 6.45-6.37 (m, 2H), 3.51 (br-s, 2H), 3.26-3.22 (m, 2H), 2.61-2.53 (m, 2H), 1.72-1.39 (m, 5H), 0.98-0.96 (d, J=5.9 Hz, 3H) ¹⁹FNMR (300 MHz, CDCl₃): −208.55 to −208.63 (dd, J₁=9 Hz, J₂=12 Hz, 1F) MS: 209.2 (M+H)⁺

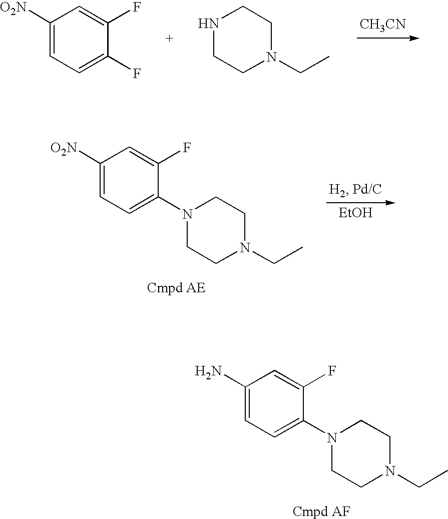

### 1-Ethyl-4-(2-fluoro-4-nitrophenyl)piperazine (Cmpd AE)

To a large round bottom flask was added acetonitrile (40 mL), 3,4-difluoronitrobenzene (0.278 mL, 2.51 mmol), and 1-ethylpiperazine (0.391 mL, 3.02 mmol). The resulting solution was refluxed (80° C.) with stirring overnight (16 h). TLC (50% EtOAc/Hexanes) showed reaction completion. CombiFlash (10 g, DCM to 10% MeOH/DCM) afforded Cmpd AE as a yellowish-orange crystalline solid. ¹HNMR (300 MHz, CDCl₃): 8.01-7.96 (ddd, J₁=0.98 Hz, J₂=2.69 Hz, J₃=9.03 Hz, 1H), 7.93-7.88 (dd, J₁=2.69 Hz, J₂=13.18 Hz, 1H), 6.94-6.89 (dd, J₁=J₂=8.79 Hz, 1H), 3.35-3.32 (t, J=5 Hz, 4H), 2.65-2.62 (t, J=5 Hz, 4H), 2.53-2.46 (q, J=7 Hz, 2H), 1.16-1.11 (t, J=7 Hz, 3H) ¹⁹FNMR (300 MHz, CDCl₃): −204.58 to −204.66 (m, 1F) MS: 254.2 (M+H)⁺

### 4-(4-Ethylpiperazin-1-yl)-3-fluoroaniline (Cmpd AF)

To a solution of Cmpd AE (599 mg, 2.365 mmol) in ethanol (60 mL) was added a Pd/C (120 mg, 0.113 mmol) catalyst. The resulting black suspension was shaken under hydrogen atmosphere (60 psi) for 2 h. TLC (10% MeOH/DCM) showed reaction completion. CombiFlash (10 g, Hexanes to EtOAc) afforded Cmpd AF as a golden brown oil. ¹HNMR (300 MHz, CDCl₃): 6.85-6.79 (dd, J₁=8.5 Hz, J₂=9.8 Hz, 1H), 6.46-6.38 (m, 2H), 3.53 (br-s, 2H), 3.04-3.00 (t, J=5 Hz, 4H), 2.62 (s, 4H), 2.52-2.44 (q, J=3 Hz, 2H), 1.14-1.10 (t, J=3 Hz, 3H) ¹⁹FNMR (300 MHz, CDCl₃): −208.65 to −208.73 (dd, J₁=9.2 Hz, J₂=12.2 Hz, 1F) GC/MS: 223 (M⁺)

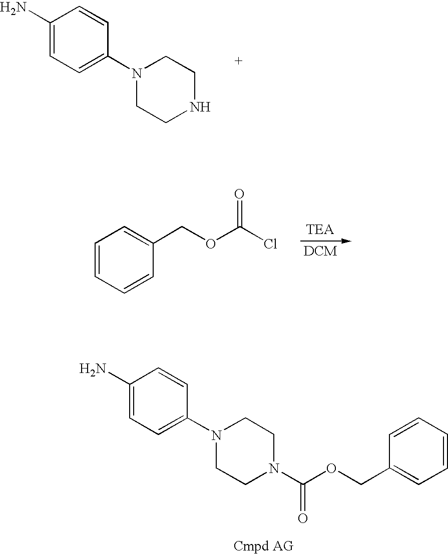

### Benzyl 4-(4-aminophenyl)piperazine-1-carboxylate (Cmpd AG)

To a dark transparent solution of 1-(4-aminophenyl)piperazine (300 mg, 1.693 mmol) and triethylamine (0.236 mL, 1.693 mmol) in DCM (30 mL) was added benzyl chloroformate (0.238 mL, 1.693 mmol) dropwise with stirring. The resulting solution was allowed to stir at rt for 1 h, after which HPLC showed reaction completion. Concentration and CombiFlash (10 g, DCM to 10% MeOH/DCM) afforded Cmpd AG as a solid. ¹HNMR (300 MHz, CDCl₃) 7.35 (m, 5H), 4.80 (d, J=8.8 Hz, 2H), 6.66 (d, J=8.8 Hz, 2H), 5.16 (d, 2H), 3.65 (m, 4H), 3.49 (br-s, 2H), 2.99 (br-s, 4H)

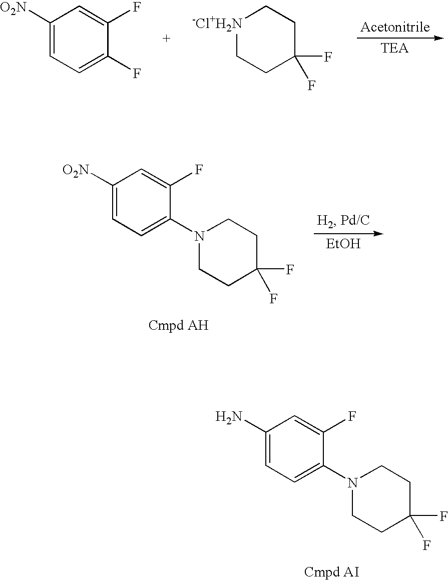

### 4,4-Difluoro-1-(2-fluoro-4-nitrophenyl)piperidine (Cmpd AH)

To a solution of 3,4-difluoronitrobenzene (0.278 mL, 2.51 mmol) in acetonitrile (40 mL) was added 4,4-difluoropiperidine hydrochloride (475 mg, 3.02 mmol) and triethylamine (0.526 mL, 3.77 mmol). TLC showed reaction completion after 18 h. Concentration and CombiFlash (10 g, Hexane to Ethyl Acetate) afforded Cmpd AH as a crystalline yellow solid. ¹HNMR (300 MHz, CDCl₃): 8.02-7.99 (d, J=9 Hz, 1H), 7.96-7.91 (d, J=12 Hz, 1H), 6.99-6.93 (dd, J1=J2=8.8 Hz, 1H), 3.43-3.39 (t, J=5 Hz, 4H), 2.23-2.11 (m, 4H) ¹⁹FNMR (300 MHz, CDCl₃): −184.33 (s, 2F), −204.68 to −204.76 (dd, J1=9 Hz, J2=12 Hz, 1F) MS (ESI+): 261.2 (M+H)+

### 4-(4,4-Difluoropiperidin-1-yl)-3-fluoroaniline (Cmpd AI)

To a solution of Cmpd AH (321 mg, 1.23 mmol) in ethanol (32 mL) was added a Pd/C (64.2 mg, 0.060 mmol) catalyst. The resulting black suspension was shaken under hydrogen atmosphere (60 psi) for 2 h. TLC (10% MeOH/DCM) showed reaction completion. CombiFlash (10 g, Hexanes to EtOAc) afforded Cmpd AI as a reddish-brown oil. ¹HNMR (300 MHz, CDCl₃): 6.85-6.82 (dd, J₁=J₂=8.3 Hz, 1H), 6.46-6.38 (m, 2H), 3.58 (br-s, 2H), 3.06 (s, 4H), 2.18-2.05 (m, 4H) ¹⁹FNMR (300 MHz, CDCl₃): −184.14 (s, 2F), −208.75 to −208.83 (dd, J₁=10.7 Hz, J₂=12.2 Hz, 1F) GC/MS: 230 (M⁺)

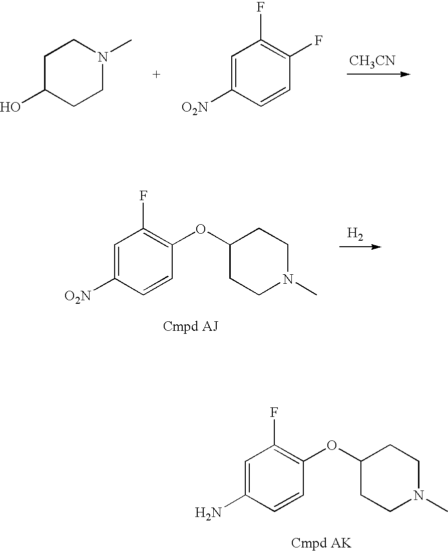

To a suspension of sodium hydride (94 mg, 3.91 mmol) in DMF (10 mL) was added 1-methyl-4-hydroxypiperidine (300 mg, 2.60 mmol) at 0° C. Hydrogen gas did not seem to evolve until solution returned to rt. The solution was allowed to stir for 1.5 h at rt. To this mixture was added 3,4-difluoronitrobenzene (0.577 mL, 5.21 mmol). The solution quickly turned a dark green, then a deep orange. This solution was allowed to stir for an additional 3 h at 150° C. Diluted in 20 mL of an aqueous half-saturated sodium bicarbonate solution and extracted into EtOAc (3×30 mL). Concentration and high-vacuum drying afforded Cmpd AJ as a crude brown liquid which would not reduce down. Will hydrogenate mixture and purify by chromatography in next step. MS confirmed structure. MS (ESI⁺): 255.2 (M+H)⁺

### 3-Fluoro-4-(1-methylpiperidin-4-yloxy)aniline (Cmpd AK)

To a solution of Cmpd AJ (2.00 g, 7.87 mmol) in ethanol (100 mL) was added a Pd/C (0.837 g, 7.87 mmol) catalyst. The black suspension was shaken under hydrogen atmosphere (60 psi) for 1 h. TLC (10% MeOH/DCM) showed reaction completion. Concentration and silica gel chromatography (10 g, DCM to 50% MeOH/DCM) afforded Cmpd AK as a minor product eluting at ˜25-30% MeOH/DCM, for a 23.5% yield across the two steps. Appearance is dark brownish oil. ¹HNMR (400 MHz, CD₃OD): 6.87-6.83 (dd, J₁=8.4 Hz, J₂=9.8 Hz, 1H), 6.50-6.46 (dd, J₁=2.5 Hz, J₂=13.3 Hz, 1H), 6.44-6.41 (ddd, J₁=1 Hz, J₂=2.5 Hz, J₃=8.4 Hz, 1H), 4.11 (m, 1H), 2.83-2.77 (m, 2H), 2.41 (m, 2H), 2.35 (s, 3H), 1.97-1.91 (m, 2H), 1.85-1.79 (m, 2H) ¹⁹FNMR (400 MHz, CD₃OD): −133.39 to −133.45 (dd, J₁=9.5 Hz, J₂=13.1 Hz, 1F) GC/MS (70 eV): 224 (M⁺)

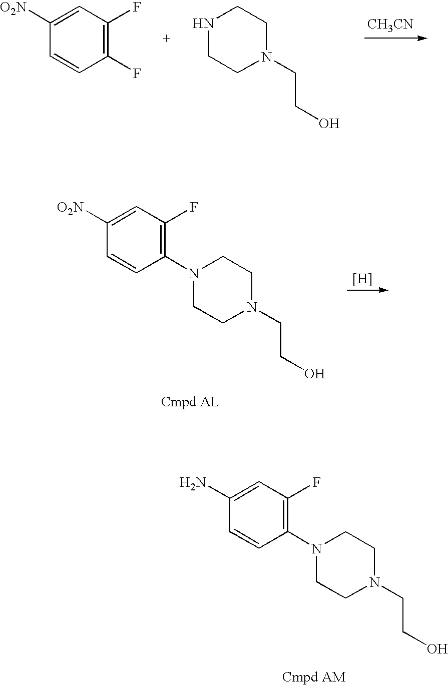

### 2-(4-(2-Fluoro-4-nitrophenyl)piperazin-1-yl)ethanol (Cmpd AL)

To a clear, colorless solution of 3,4-difluoronitrobenzene (0.221 mL, 1.999 mmol) in acetonitrile (20 mL) was added 1-piperazineethanol (0.245 mL, 1.99 mmol). The solution immediately changed to a deep yellow color. The reaction solution was heated for 1 h at 130° C., after which the solution became cloudy and turned to a deep orange color. Concentrated to dryness, applied to silica gel, and purified via chromatography (10 g, DCM to 15% MeOH/DCM) to afford Cmpd AL (464 mg, 1.723 mmol, 86% yield). ¹HNMR (400 MHz, CDCl₃): 8.01-7.98 (dd, J₁=2.5 Hz, J₂=9 Hz, 1H), 7.93-7.89 (dd, J₁=2.5 Hz, J₂=13.1 Hz, 1H), 6.94-6.90 (dd, J₁=J₂=8.8 Hz, 1H), 3.69-3.66 (t, J=5.3 Hz, 2H), 3.34-3.32 (m, 4H), 2.72-2.69 (m, 4H), 2.65-2.62 (t, J=5.3 Hz, 2H) ¹⁹FNMR (400 MHz, CDCl₃): −118.5 (dd, J₁=8.3 Hz, J₂=13.1 Hz, 1F) MS (ESI⁺): 270.3 (M+H)⁺

### 2-(4-(4-Amino-2-fluorophenyl)piperazin-1-yl)ethanol (Cmpd AM)

To an orange transparent solution of Cmpd AL (460 mg, 1.708 mmol) in ethanol (45 mL) was added a Pd/C (92 mg, 0.086 mmol) catalyst. The reaction mixture was shaken under hydrogen atmosphere (60 psi) for 1 h. Concentration and silica gel chromatography (10 g, DCM to 50% MeOH/DCM) afforded Cmpd AM as a yellowish crystalline solid. ¹HNMR (400 MHz, CDCl₃): 6.82-6.78 (dd, J₁=8.4 Hz, J₂=9.6 Hz, 1H), 6.45-6.38 (m, 2H), 3.66-3.63 (t, J=5.4 Hz, 2H), 3.48 (s, 1H), 3.01-3.00 (m, 4H), 2.70-2.69 (m, 4H), 2.62-2.60 (t, J=5.4 Hz, 2H) ¹⁹FNMR (400 MHz, CDCl₃): −122.7 (dd, J₁=10 Hz, J₂=13 Hz, 1F) MS (ESI⁺): 240.2 (M+H)⁺

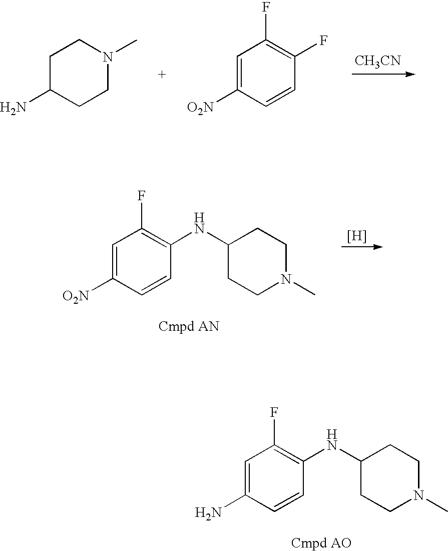

### N-(2-fluoro-4-nitrophenyl)-1-methylpiperidin-4-amine (Cmpd AN)

To a solution of 4-amino-1-methylpiperidine (0.220 mL, 1.751 mmol) in acetonitrile (20 mL) was added 3,4-difluoronitrobenzene (0.194 mL, 1.751 mmol). The reaction mixture was heated at 130° C. via microwave irradiation for 30 min. TLC showed a new spot. Concentration and silica gel chromatography (4 g, DCM to 10% MeOH/DCM) afforded Cmpd AN as a slightly yellow crystalline solid. ¹HNMR (400 MHz, CDCl₃): 7.94-7.93 (dd, J₁=2.3 Hz, J₂=9 Hz, 1H), 7.83-7.80 (dd, J₁=2.3 Hz, J₂=11.7 Hz, 1H), 6.62-6.58 (dd, J₁=8.4 Hz, J₂=9 Hz, 1H), 4.58-4.57 (d, J=3.9 Hz, 1H), 3.39-3.37 (m, 1H), 2.81-2.78 (m, 2H), 2.27 (s, 3H), 2.15-2.10 (m, 2H), 2.05-2.01 (m, 2H), 1.62-1.53 (m, 2H) ¹⁹FNMR (400 MHz, CDCl₃): −135.05 to −135.11 (m, 1F) GC/MS (70 eV): 253 (M⁺)

### 2-Fluoro-N-1-(1-methylpiperidin-4-yl)benzene-1,4-diamine (Cmpd AO)

To a solution of Cmpd AN (155 mg, 0.612 mmol) in EtOH (30 mL) was added a Pd/C (31 mg, 0.029 mmol) catalyst. The resulting black suspension was shaken under hydrogen atmosphere (60 psi) for 2 h. TLC (10% MeOH/DCM) showed reaction completion. Concentration and silica gel chromatography (4 g, DCM to 50% MeOH/DCM) afforded Cmpd AO as a slightly yellowish powdered solid. ¹HNMR (400 MHz, CD₃OD): 6.75-6.70 (dd, J₁=8.6 Hz, J₂=9.2 Hz, 1H), 6.51-6.45 (m, 2H), 3.43-3.38 (m, 3H), 3.02-2.96 (m, 2H), 2.78 (s, 3H), 2.18-2.13 (m, 2H), 1.70-1.67 (m, 2H) ¹⁹FNMR (400 MHz, CD₃OD): −133.16 (m, 1F)

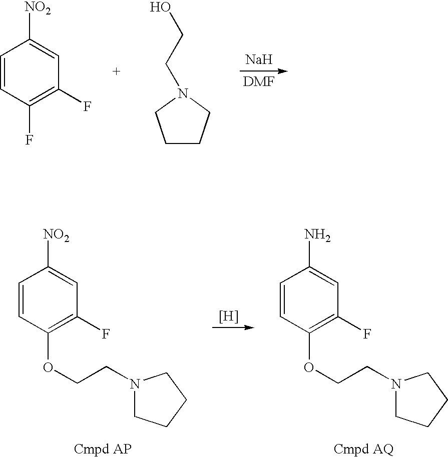

### 1-(2-(2-Fluoro-4-nitrophenoxy)ethyl)pyrrolidine (Cmpd AP)

To the mixture of pyrrolidin ethanol (0.724 g, 6.29 mmol) was added NaH (1.2 eq). After 5 min, difluoro-nitro-benzene (1 g, 6.29 mmol) was added. Then, 30 min later, one more eq of NaH was added. The mixture was diluted with EtOAc and water. Extraction with EtOAc (80 mL×3) and washed with 50 mL of water. The organic phase was dried by MgSO₄ and concentration for combiflash (24 g, DCM to 10% MeOH/DCM) gave a yellow solid. ¹H NMR (400 MHz, CDCl₃) 8.02 (dd, J₁=1.47 Hz, J₂=9.20 Hz, 1H), 7.98 (dd, J₁=2.74 Hz, J₂=10.76 Hz, 1H), 7.02 (t, J=8.22 Hz, 1H), 4.26 (t, J=5.87 Hz, 2H), 2.96 (t, J=5.87 Hz, 2H), 2.64 (m, 4H), 1.80 (m, 4H).

### 3-Fluoro-4-(2-(pyrrolidin-1-yl)ethoxy)aniline (Cmpd AQ)

The mixture of Cmpd AP (550 mg, 2.16 mmol), Pd/C (100 mg) in EtOH (50 mg) was shaken under 60 psi for 3 h. TLC (10% MeOH/DCM) showed reaction completed. The mixture was concentrated for combiflash (10 g, DCM to 10% MeOH/DCM) afforded a yellow oil. ¹H-NMR (400 Mhz, CD₃OD) 6.84 (t, J=9.19 Hz, 1H), 6.47 (dd, J₁=2.73 Hz, J₂=13.1 Hz, 1H), 6.40 (m, 1H), 4.05 (t, J=5.67 Hz, 2H), 2.86 (t, J=5.67 Hz, 2H), 2.66 (m, 4H), 1.81 (m, 4H). ¹⁹FNMR (400 Mhz, CD₃OD) −135.0

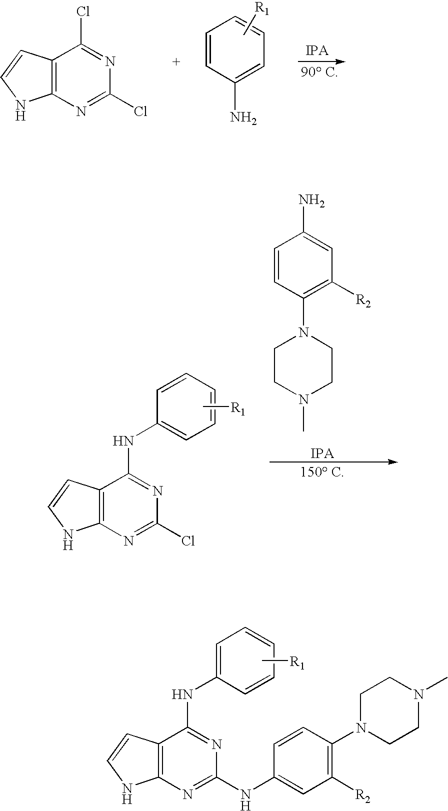

2-(3-(2-(4-(4-Ethylpiperazin-1-yl)-3-fluorophenylamino)-7H-pyrrolo[2,3-d]pyrimidin-4-ylamino)phenyl)acetonitrile (EXAMPLE 17): ¹HNMR (MHz, CD₃OD): 7.98-7.97 (dd, 1H), 7.79-7.75 (dd, 1H), 7.73-7.71 (ddd, 1H), 7.35-7.31 (dd, 1H), 7.21-7.19 (ddd, 1H), 7.04-7.02 (d, 1H), 6.97-6.92 (dd, 1H), 6.88-6.87 (d, 1H), 6.59-6.58 (d, 1H), 3.34 (s, 2H), 3.07 (s, 4H), 2.67 (s, 4H), 2.54-2.49 (q, 2H), 1.16-1.13 (t, 3H) ¹⁹FNMR (MHz, CD₃OD): −121.52 to −121.59 (dd, 1F) MS (ESI⁺): 471.4 (M+H)⁺; 493.3 (M+Na)⁺

2-(3-(2-(4-(4-(Methylsulfonyl)piperazin-1-yl)phenylamino)-7H-pyrrolo[2,3-d]pyrimidin-4-ylamino)phenyl)acetonitrile (EXAMPLE 19): ¹HNMR (400 MHz, DMSO-d₆): 11.17 (s, 1H), 9.19 (s, 1H), 8.57 (s, 1H), 8.01-7.99 (d, 1H), 7.89 (s, 1H), 7.65-7.63 (d, 2H), 7.34-7.30 (dd, 1H), 6.96-6.88 (m, 3H), 6.65 (s, 1H), 4.02 (s, 2H), 3.26-3.24 (t, 4H), 3.16-3.13 (t, 4H), 2.92 (s, 3H) MS (ESI⁺): 503.2 (M+H)⁺; 525.4 (M+Na)⁺

2-(3-(2-(4-(4,4-Difluoropiperidin-1-yl)-3-fluorophenylamino)-7H-pyrrolo[2,3-d]pyrimidin-4-ylamino)phenyl)acetonitrile (EXAMPLE 21): ¹HNMR (MHz, CD₃OD): 7.98-7.97 (dd, 1H), 7.81-7.76 (dd, 1H), 7.74-7.71 (ddd, 1H), 7.35-7.31 (dd, 1H), 7.20-7.17 (ddd, 1H), 7.05-7.02 (ddd, 1H), 7.00-6.95 (dd, 1H), 6.88-6.87 (d, 1H), 6.59-6.58 (d, 1H), 3.34 (s, 2H), 3.13-3.11 (t, 4H), 2.17-2.07 (m, 4H) ¹⁹FNMR (MHz, CD₃OD): −96.85 (s, 2F), −121.70 to −121.77 (dd, 1F) MS (ESI⁺): 478.4 (M+H)⁺; 500.3 (M+Na)⁺

2-(3-(2-(4-Morpholinophenylamino)-7H-pyrrolo[2,3-d]pyrimidin-4-ylamino)phenyl)acetonitrile (EXAMPLE 23): (400 MHz, (CD₃)₂CO) 8.06 (s, 1H), 788 (m, 1H), 7.68 (d, J=6.8 Hz, 2H), 7.30 (t, J=7.6 Hz, 1H), 7.00 (d, J=7.6 Hz, 1H), 6.92 (m, 3H), 6.57 (d, J=4 Hz, 1H), 3.90 (s, 2H), 3.77 (m, 4H), 3.06 (m, 4H), ESI: 426.4 (M+H)⁺

2-(3-(2-(4-(2-(Pyrrolidin-1-yl)ethoxy)phenylamino)-7H-pyrrolo[2,3-d]pyrimidin-4-ylamino)phenyl)acetonitrile (EXAMPLE 18): ¹HNMR (400 MHz, CD₃OD): 7.96-7.95 (m, 1H), 7.69-7.66 (ddd, J₁=0.98 Hz, J₂=2.15 Hz, J₃=8.22 Hz, 1H), 7.51-7.49 (dd, J₁=Hz, J₂=Hz, 2H), 7.30-7.26 (dd, J₁=J2=7.8 Hz, 1H), 7.00-6.98 (ddd, J1=0.97 Hz, J₂=1.57 Hz, J₃=7.63 Hz, 1H), 6.87-6.83 (m, 3H), 6.57-6.56 (d, J=3.5 Hz, 1H), 4.10-4.08 (t, J=5.67 Hz, 2H), 2.92-2.89 (t, J=5.67 Hz, 2H), 2.69-2.66 (m, 4H), 1.84-1.81 (m, 4H) MS (ESI⁺): 456.4 (M+H)⁺; 478.4 (M+Na)⁺

2-(3-(2-(4-(2-(Dimethylamino)ethoxy)phenylamino)-7H-pyrrolo[2,3-d]pyrimidin-4-ylamino)phenyl)acetonitrile (EXAMPLE 20): ¹HNMR (400 MHz, CDCl₃): 7.84 (s, 1H), 7.51-7.46 (m, 3H), 7.34-7.30 (dd, 1H), 7.03-7.01 (d, 1H), 6.89-6.87 (d, 2H), 6.75-6.72 (m, 2H), 6.22 (d, 1H), 4.08-4.05 (t, 2H), 3.71 (s, 2H), 2.76-2.73 (t, 2H), 2.35 (s, 6H) MS (ESI⁺): 428.4 (M+H)⁺; 450.2 (M+Na)⁺

2-(3-(2-(4-(4-(2-Hydroxyethyl)piperazin-1-yl)phenylamino)-7H-pyrrolo[2,3-d]pyrimidin-4-ylamino)phenyl)acetonitrile (EXAMPLE 22): ¹HNMR (400 MHz, DMSO-d6): 11.15 (s, 1H), 9.17 (s, 1H), 8.50 (s, 1H), 8.00-7.98 (d, 1H), 7.91 (s, 1H), 7.60-7.58 (d, 2H) 7.33-7.29 (dd, 1H), 6.96-6.94 (d, 1H), 6.86-6.84 (m, 3H), 6.47 (s, 1H), 4.42 (s, 1H), 4.03-3.99 (t, 2H), 3.52 (s, 2H), 3.03 (s, 4H), 2.55 (s, 4H), 2.44-2.41 (t, 2H) MS (ESI⁺): 471.4 (M+H)⁺; 493.4 (M+Na)⁺

Other EXAMPLES of the present invention are shown in the following table:

| EX. | Structure |
|---|---|
| 24 | 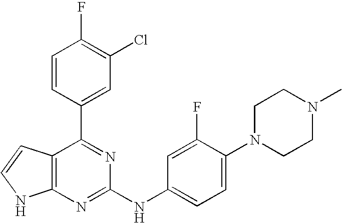 |
| 25 | 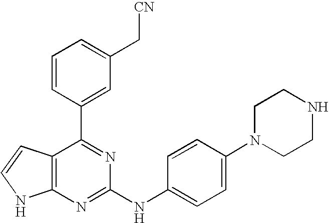 |
| 26 | 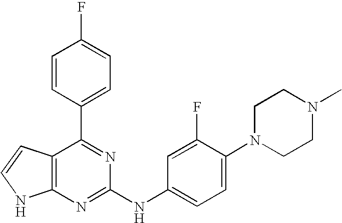 |
| 27 | 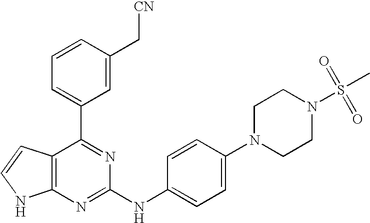 |
| 28 | 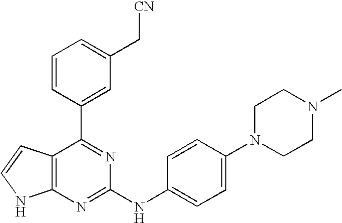 |
| 29 | 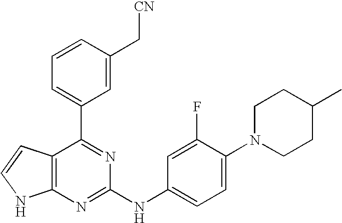 |
| 30 | 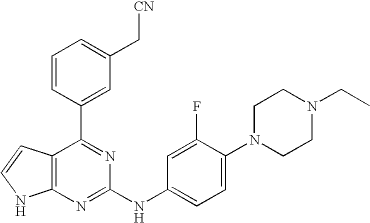 |
| 31 | 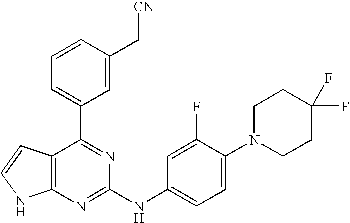 |
| 32 | 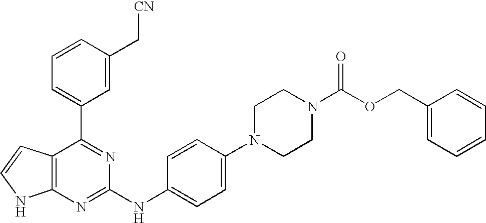 |
| 33 | 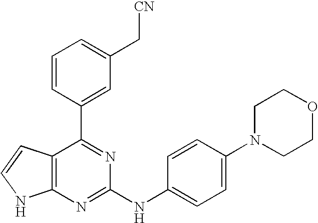 |
| 34 | 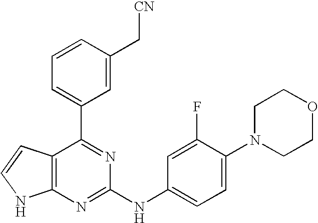 |
| 35 | 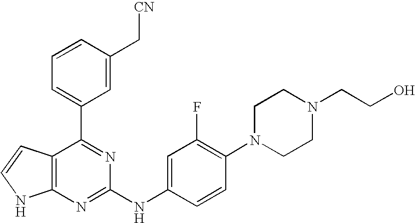 |
| 36 | 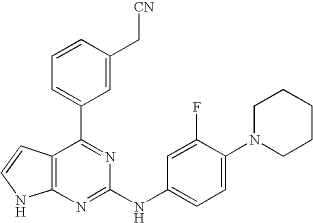 |
| 37 | 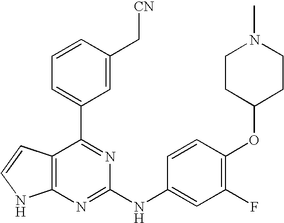 |
| 38 | 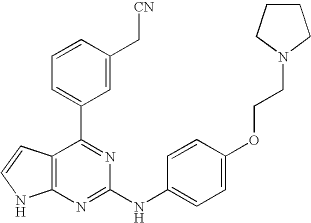 |
| 39 | 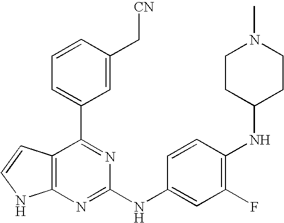 |
| 40 | 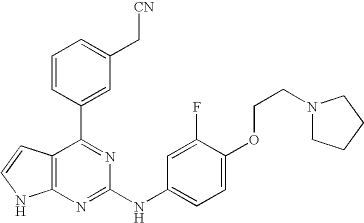 |
| 41 | 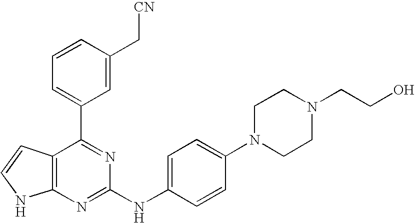 |
| 42 | 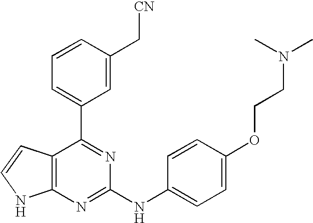 |
| 43 | 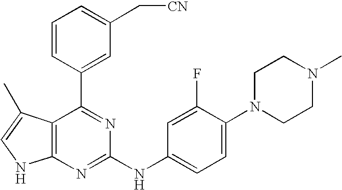 |
| 44 | 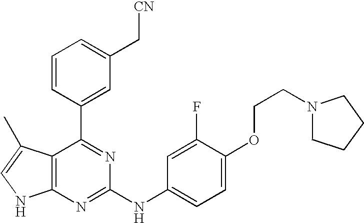 |
| 45 | 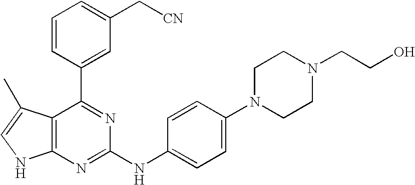 |
| 46 | 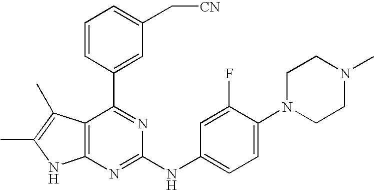 |
| 47 | 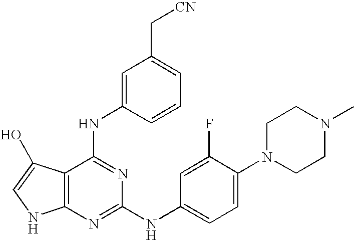 |
| 49 | 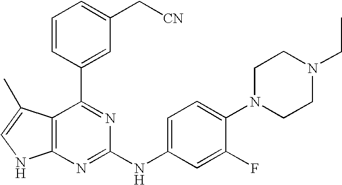 |

The above EXAMPLES may be made by the following scheme and those shown above, including the use of intermediate Cmpds BA, BB. BC, and BD below.

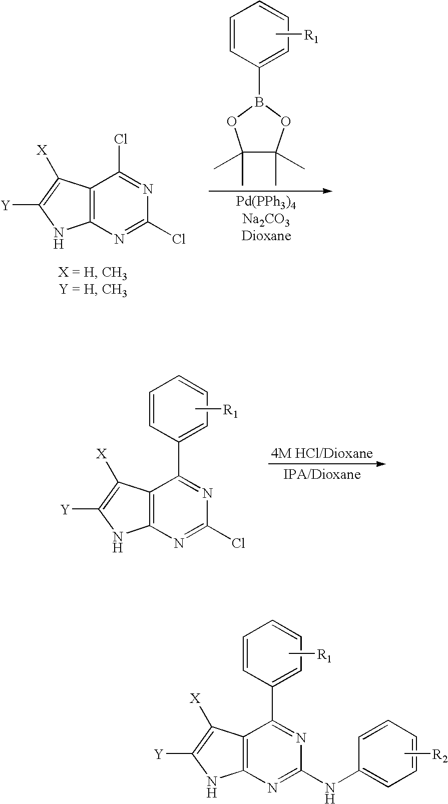

### 2-Chloro-4-(3-chloro-4-fluorophenyl)-7H-pyrrolo[2,3-d]pyrimidine (Cmpd BA)

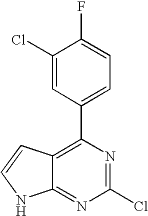

The reaction mixture containing 2,4-dichloro-7H-pyrrolo[2,3-d]pyrimidine (250 mg, 1.33 mmol), 3-chloro-4-fluorophenylboronic acid (232 mg, 1.33 mmol), Pd(PPh₃)₄ (30.7 mg, 0.02 eq) and Na₂CO₃ (1.33 mL, 2M) in dioxane (5 mL) was heat by microwave at 120° C. for 1 h. HPLC showed reaction completed. The reaction mixture was concentrated and combiflash (24 g, Hexane to EtOAc) afforded 304 mg of a pale yellow solid. ¹H-NMR (400 MHz, (CH₃)₂CO) 8.34 (m, 1H), 8.24 (m, 1H), 7.71 (d, J=3.72 Hz, 1H), 7.54 (t, J=8.81 Hz, 1H), 7.01 (d, J=3.72 Hz, 1H), ESI (NEG): 280.0 (M−H)⁻

### 2-Chloro-4-(4-fluorophenyl)-7H-pyrrolo[2,3-d]pyrimidine (Cmpd BB)

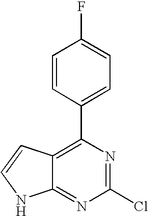

¹H-NMR (400 MHz, (CH₃)₂CO) 8.30 (m, 2H), 8.24 (m, 1H), 7.67 (d, J=3.72 Hz, 1H), 7.36 (t, J=8.80 Hz, 2H), 6.98 (d, J=3.72 Hz, 1H) ESI: 248.2 (M+H)⁺

4-(3-Chloro-4-fluorophenyl)-N-(3-fluoro-4-(4-methylpiperazin-1-yl)phenyl)-7H-pyrrolo[2,3-d]pyrimidin-2-amine (EXAMPLE 24): ¹H-NMR (400 MHz, CD₃OD) 8.28 (d, J=7.24 Hz, 1H), 8.12 (m, 1H), 7.90 (dd, J₁=15.46 Hz, J₂=2.35 Hz, 1H), 7.42 (t, J=8.8 Hz, 1H), 7.22 (d, J=8.8 Hz, 1H), 7.18 (d, J=3.72 Hz, 1H), 6.99 (t, J=9.19 Hz, 1H), 6.66 (d, J=3.72 Hz, 1H), 3.07 (s, 4H), 2.67 (s, 4H), 2.37 (s, 3H). ¹⁹F-NMR (400 MHz, CD₃OD) −116.16, −1124.2 ESI-MS: 455.4 (M+H)⁺

N-(3-fluoro-4-(4-methylpiperazin-1-yl)phenyl)-4-(4-fluorophenyl)-7H-pyrrolo[2,3-d]pyrimidin-2-amine (EXAMPLE 26): ¹H-NMR (400 MHz, CD₃OD) 8.15 (m, 2H), 8.12 (m, 1H), 7.90 (d, J=15.45 Hz, 1H), 7.24 (m, 3H), 7.13 (m, 1H), 6.95 (d, J=9.00 Hz, 1H), 6.63 (d, J=3.32 Hz, 1H), 3.04 (s, 4H), 2.63 (s, 4H), 2.34 (s, 3H) ¹⁹F-NMR (400 MHz, CD₃OD) −113.34, −1124.1, ESI-MS: 421.4 (M+H)⁺

2-(3-(2-(4-(4-Methylpiperazin-1-yl)phenylamino)-7H-pyrrolo[2,3-d]pyrimidin-4-yl)phenyl)acetonitrile (EXAMPLE 28): ¹HNMR (300 MHz, CD₃OD) 8.16 (s, 1H), 8.08 (d, J=7.6 Hz, 1H), 7.69 (d, J=9.0 Hz, 2H), 7.54 (m, 2H), 7.14 (d, J=3.68 Hz, 1H), 6.98 (d, J=9.0 Hz, 2H), 6.67 (d, J=3.66 Hz, 1H), 4.04 (s, 1H), 3.15 (m, 4H), 2.64 (m, 4H), 2.35 (s, 3H). ESI: 425.4 (100%), 424.4 (50%)

2-(3-(2-(4-(4-Ethylpiperazin-1-yl)-3-fluorophenylamino)-7H-pyrrolo[2,3-d]pyrimidin-4-yl)phenyl)acetonitrile (EXAMPLE 30): ¹HNMR (300 MHz, CD₃OD) 8.13 (s, 1H), 8.06 (d, J=7.57 Hz, 1H), 7.88 (d, J=15.4 Hz, 1H), 7.47 (m, 2H), 7.32 (d, J=8.79 Hz, 1H), 7.15 (d, J=3.67 Hz, 1H), 6.97 (t, J=9.52 Hz, 1H), 6.66 (d, J=3.67 Hz, 1H), 4.01 (s, 2H), 3.04 (s, 4H), 2.63 (s, 4H), 2.48 (q, J=7.33 Hz, 2H), 1.11 (t, J=7.32 Hz, 3H) ¹⁹FNMR (300 MHz, CD₃OD) −206.16, MS-ESI: 456.4 (M+H)⁺

Benzyl 4-(4-(4-(3-(cyanomethyl)phenyl)-7H-pyrrolo[2,3-d]pyrimidin-2-ylamino)phenyl)piperazine-1-carboxylate (EXAMPLE 32): ¹HNMR (300 MHz, CD₃OD): 8.17-8.09 (m, 2H), 7.72-7.69 (d, J=8.8 Hz, 2H), 7.58-7.52 (m, 2H), 7.37 (m, 5H), 7.16 (s, 1H), 7.01-6.98 (d, J=8.8 Hz, 2H), 6.68 (s, 1H), 5.15 (s, 2H), 3.65 (s, 4H), 3.07 (s, 4H) MS: 546.4 (M+H)⁺

2-(3-(2-(4-(piperazin-1-yl)phenylamino)-7H-pyrrolo[2,3-d]pyrimidin-4-yl)phenyl)acetonitrile (EXAMPLE 25): To a small, clean reaction flask was added EXAMPLE 32 (161 mg, 0.296 mmol) and an HBr/HOAc solution (1 mL, 5.52 mmol). Upon addition of the HBr solution, evolution of carbon dioxide was apparent. The reaction mixture was allowed to stir for 1 h. Diethyl Ether was added to precipitate product and the product was isolated via filtration. Concentration and CombiFlash (4 g, DCM to 10% MeOH/DCM) afforded EXAMPLE 25 as a powdered solid. ¹HNMR (400 MHz, CD₃OD): 8.18 (s, 1H), 8.10-8.08 (d, 1H), 7.75-7.73 (d, 2H), 7.59-7.55 (dd, 1H), 7.50-7.48 (d, 1H), 7.16-7.15 (d, 1H), 7.03-7.01 (d, 2H), 6.69-6.68 (d, 1H), 4.04 (s, 2H) MS (ESI⁺): 410.4 (M+H)⁺; 431.3 (M+Na)⁺

2-(3-(2-(4-(4-(Methylsulfonyl)piperazin-1-yl)phenylamino)-7H-pyrrolo[2,3-d]pyrimidin-4-yl)phenyl)acetonitrile (EXAMPLE 27): ¹HNMR (400 MHz, DMSO-d₆): 11.60 (s, 1H), 9.13 (s, 1H), 8.12 (s, 1H), 8.09-8.07 (d, 1H), 7.76-7.74 (d, 2H), 7.62-7.58 (dd, 1H), 7.51-7.49 (d, 1H), 7.26-7.25 (d, 1H), 6.95-6.93 (d, 2H), 6.68-6.67 (d, 1H), 4.20 (s, 2H), 3.25-3.21 (t, 4H), 3.16-3.13 (t, 4H), 2.92 (s, 3H) MS (ESI⁺): 488.2 (M+H)⁺; 510.3 (M+Na)⁺

2-(3-(2-(3-Fluoro-4-(4-methylpiperidin-1-yl)phenylamino)-7H-pyrrolo[2,3-d]pyrimidin-4-yl)phenyl)acetonitrile (EXAMPLE 29): ¹HNMR (300 MHz, CDCl₃): 8.18-8.17 (dd, J₁=J₂=1.7 Hz, 1H), 8.13-8.09 (ddd, J₁=1.2 Hz, J₂=1.7 Hz, J₃=7.6 Hz, 1H), 7.94-7.88 (dd, J₁=2.4 Hz, J₂=15.4 Hz, 1H), 7.61-7.56 (dd, J₁=7.8 Hz, J₂=7.6 Hz, 1H), 7.53-7.50 (ddd, J₁=1.2 Hz, J₂=1.7 Hz, J₃=7.8 Hz, 1H), 7.37-7.33 (ddd, J₁=0.98 Hz, J₂=Hz, J₃=8.8 Hz, 1H), 7.20-7.19 (d, J=3.66 Hz, 1H), 7.06-6.99 (dd, J₁=9.5 Hz, J₂=9.0 Hz, 1H), 6.71-6.70 (d, J=3.66 Hz, 1H), 2.69-2.62 (dd, J₁=10.5 Hz, J₂=11.5 Hz, 2H), 2.01 (s, 2H), 1.78-1.74 (m, 2H), 1.47-1.39 (m, 3H), 1.28-1.18 (m, 2H), 1.01-0.99 (d, J=6.1 Hz, 3H) ¹⁹FNMR (300 MHz, CDCl₃): −206.18 to −206.27 (dd, J₁=10.68 Hz, J₂=15.26 Hz, 1F) MS: 443.3

2-(3-(2-(4-(4,4-Difluoropiperidin-1-yl)-3-fluorophenylamino)-7H-pyrrolo[2,3-d]pyrimidin-4-yl)phenyl)acetonitrile (EXAMPLE 31): ¹HNMR (300 MHz, CD₃OD): 8.16-8.09 (m, 2H), 7.95-7.90 (d, J=15 Hz, 1H), 7.60-7.51 (m, 2H), 7.37-7.34 (d, J=8.06 Hz, 1H), 7.19-7.18 (m, 1H), 7.05-6.99 (dd, J₁=8.8 Hz, J₂=9.0 Hz, 1H), 6.70-6.69 (m, 1H), 3.13 (s, 4H), 2.13 (m, 4H) ¹⁹FNMR (300 MHz, CD₃OD): −181.62 (s, 2F), −206.39 to −206.48 (dd, J₁=15 Hz, J₂=9 Hz, 1F) MS (ESI⁺): 465.3 (M+H)⁺

2-(3-(2-(4-Morpholinophenylamino)-7H-pyrrolo[2,3-d]pyrimidin-4-yl)phenyl)acetonitrile (EXAMPLE 33): ¹HNMR (300 MHz, CD₃OD) 8.04 (s, 1H), 8.00 (d, J=7.8 Hz, 1H), 7.60 (d, J=9.0 Hz, 2H), 7.53 (1H, 7.8 Hz, 1H), 7.45 (m, 1H), 7.03 (d, J=3.67 Hz, 1H), 6.93 (d, J=9.0 Hz, 2H), 6.58 (d, J=3.66 Hz, 1H), 4.48 (s, 4H), 3.91 (s, 2H), 3.08 (m, 4H) ESI: 411.3 (M+H)⁺

2-(3-(2-(3-Fluoro-4-morpholinophenylamino)-7H-pyrrolo[2,3-d]pyrimidin-4-yl)phenyl)acetonitrile (EXAMPLE 34): ¹HNMR (300 MHz, CDCl₃): 8.15 (s, 1H), 8.11-8.08 (d, J=8.06 Hz, 1H), 7.93-7.88 (d, J=15.38 Hz, 1H), 7.59-7.48 (m, 2H), 7.37-7.34 (d, J=8.79 Hz, 1H), 7.18-7.17 (d, J=3.66 Hz, 1H), 7.01-6.95 (dd, J₁=9.03 Hz, J₂=9.28 Hz, 1H), 6.69-6.68 (d, J=3.66 Hz, 1H), 3.85-3.82 (t, J=4 Hz, 4H), 3.35 (s, 2H), 3.02-3.00 (t, J=4 Hz, 4H) ¹⁹FNMR (300 MHz, CDCl₃): −206.50 to −206.59 (dd, J₁=10.68 Hz, J₂=15.26 Hz, 1F) MS (ESI⁺): 429.3 (M+H)⁺

2-(3-(2-(3-Fluoro-4-(piperidin-1-yl)phenylamino)-7H-pyrrolo[2,3-d]pyrimidin-4-yl)phenyl)acetonitrile (EXAMPLE 36): ¹HNMR (300 MHz, CD₃OD): 8.16 (m 1H), 8.10 (m, 1H), 7.94-7.88 (dd, J₁=2.44 Hz, J₂=15.4 Hz, 1H), 7.60-7.55 (m, 1H), 7.52-7.49 (m, 1H), 7.67-7.33 (m, 1H), 7.19-7.18 (d, J=3.66 Hz, 1H), 7.05-6.98 (m, 1H), 6.70-6.69 (d, J=3.66 Hz, 1H), 2.98-2.94 (t, J=4.88 Hz, 4H), 1.77-1.75 (m, 4H), 1.62-1.58 (m, 2H) ¹⁹FNMR (300 MHz, CD₃OD): −206.28 to −206.37 (dd, J₁=10.68 Hz, J₂=15.26 Hz, 1F) MS: 429.3 (M+H)⁺

2-(3-(2-(4-(2-(Pyrrolidin-1-yl)ethoxy)phenylamino)-7H-pyrrolo[2,3-d]pyrimidin-4-yl)phenyl)acetonitrile (EXAMPLE 38): ¹HNMR (400 MHz, CD₃OD): 8.14 (s, 1H), 8.06 (s, 1H), 7.67 (s, 2H), 7.54 (s, 1H), 7.47 (s, 1H), 7.12 (s, 1H), 6.90 (s, 2H), 6.65 (s, 1H), 4.10 (s, 2H), 4.02 (s, 2H), 2.93 (s, 2H), 2.71 (s, 4H), 1.83 (s, 4H) MS (ESI⁺): 439.2 (M+H)⁺

2-(3-(2-(3-Fluoro-4-(2-(pyrrolidin-1-yl)ethoxy)phenylamino)-7H-pyrrolo[2,3-d]pyrimidin-4-yl)phenyl)acetonitrile (EXAMPLE 40): ¹HNMR (400 MHz, CD₃OD) 8.14 (s, 1H), 8.09 (d, J=6.06 Hz, 1H), 7.90 (d, J=14.3 Hz, 1H), 7.55 (m, 1H), 7.54 (m, 1H), 7.33 (d, J=8.4 Hz, 1H), 7.16 (s, 1H), 7.01 (m, 1H), 6.67 (s, 1H), 4.14 (m, 2H), 4.02 (s, 2H), 2.89 (m, 2H), 2.67 (s, 4H), 1.18 (s, 4H). ¹⁹F NMR (400 MHz, CD₃OD) −135.833, 135.858 ESI-MS: 457.4 (M+H)⁺

2-(3-(2-(4-(2-(Dimethylamino)ethoxy)phenylamino)-7H-pyrrolo[2,3-d]pyrimidin-4-yl)phenyl)acetonitrile (EXAMPLE 42): ¹HNMR (400 MHz, CD₃OD): 8.16 (s, 1H), 8.10-8.08 (d, J=7.6 Hz, 1H), 7.70-7.67 (d, J=9 Hz, 2H), 7.59-7.55 (dd, J₁=J₂=7.6 Hz, 1H), 7.51-7.49 (d, J=7.6 Hz, 1H), 7.15-7.14 (d, J=3.7 Hz, 1H), 6.93-6.91 (d, J=9 Hz, 2H), 6.67-6.66 (d, J=3.7 Hz, 1H), 4.11-4.08 (t, J=5 Hz, 2H), 4.04 (s, 2H), 2.78-2.76 (t, J=5 Hz, 2H), 2.35 (s, 6H) MS (ESI⁺): 413.3 (M+H)⁺

2-(3-(2-(3-Fluoro-4-(4-(2-hydroxyethyl)piperazin-1-yl)phenylamino)-7H-pyrrolo[2,3-d]pyrimidin-4-yl)phenyl)acetonitrile (EXAMPLE 35): ¹HNMR (400 MHz, CD₃OD): 8.17 (s, 1H), 8.12-8.10 (d, J=7.8 Hz, 1H), 7.93-7.89 (dd, J₁=2.4 Hz, J₂=15 Hz, 1H), 7.60-7.52 (dd, J₁=J₂=7.6 Hz, 1H), 7.51-7.50 (d, J=7.8 Hz, 1H), 7.38-7.35 (d, J=8.6 Hz, 1H), 7.19 (d, J=3.5 Hz, 1H), 7.03-6.98 (dd, J₁=9.8 Hz, J₂=8.8 Hz, 1H), 6.70-6.69 (d, J=3.7 Hz, 1H), 4.05 (s, 2H), 3.75-3.72 (t, J=6 Hz, 2H), 3.08 (m, 4H), 2.73 (m, 4H), 2.63-2.60 (t, J=6 Hz, 2H) ¹⁹FNMR (400 MHz, CD₃OD): −124.14 (dd, J₁=10.1 Hz, J₂=15.5 Hz, 1F) MS (ESI⁺): 472.4 (M+H)⁺; 494.4 (M+Na)⁺

2-(3-(2-(3-Fluoro-4-(1-methylpiperidin-4-yloxy)phenylamino)-7H-pyrrolo[2,3-d]pyrimidin-4-yl)phenyl)acetonitrile (EXAMPLE 37): ¹HNMR (400 MHz, CD₃OD): 8.16 (s, 1H), 8.11-8.09 (d, J=7.6 Hz, 1H), 7.95-7.90 (dd, J₁=2.5 Hz, J₂=14 Hz, 1H), 7.59-7.55 (dd, J₁=7.6 Hz, J₂=7.4 Hz, 1H), 7.51-7.49 (d, J=8.2 Hz, 1H), 7.35-7.32 (m, 1H), 7.19-7.18 (d, J=3.71 Hz, 1H), 7.05-7.00 (dd, J₁=9.2 Hz, J₂=9 Hz, 1H), 6.69 (d, J=3.5 Hz, 1H), 4.24 (m, 1H), 4.04 (s, 2H), 2.74 (m, 2H), 2.34 (m, 2H), 2.29 (s, 3H), 2.01-1.89 (m, 2H), 1.87-1.80 (m, 2H) ¹⁹FNMR (400 MHz, CD₃OD): −133.17 to −133.24 (dd, J₁=9.5 Hz, J₂=14.3 Hz, 1F) MS (ESI⁺): 457.4 (M+H)⁺

2-(3-(2-(3-Fluoro-4-(1-methylpiperidin-4-ylamino)phenylamino)-7H-pyrrolo[2,3-d]pyrimidin-4-yl)phenyl)acetonitrile (EXAMPLE 39): ¹HNMR (400 MHz, CD₃OD): 8.15 (s, 1H), 8.10-8.08 (d, J=7.63 Hz, 1H), 7.79-7.74 (dd, J₁=2.5 Hz, J₂=14.3 Hz, 1H), 7.59-7.55 (dd, J₁=7.6 Hz, J₂=7.8 Hz, 1H), 7.52-7.49 (m, 1H), 7.27-7.24 (m, 1H), 7.16-7.15 (d, J=3.72 Hz, 1H), 6.83-6.79 (dd, J₁=J₂=9.2 Hz, 1H), 6.67-6.66 (d, J=3.72 Hz, 1H), 4.04 (s, 2H), 2.93-2.90 (m, 2H), 2.33 (s, 3H), 2.28-2.25 (m, 2H), 2.06-2.02 (m, 2H), 1.56-1.53 (m, 2H) ¹⁹FNMR (400 MHz, CD₃OD): −134.65 to −134.72 (m, 1F) MS (ESI⁺): 456.2 (M+H)⁺; 478.4 (M+Na)⁺

2-(3-(2-(4-(4-(2-Hydroxyethyl)piperazin-1-yl)phenylamino)-7H-pyrrolo[2,3-d]pyrimidin-4-yl)phenyl)acetonitrile (EXAMPLE 41): ¹HNMR (400 MHz, (CD₃)₂CO): 10.64 (br-s, 1H), 8.26 (s, 1H), 8.21 (s, 1H), 8.18-8.16 (d, J=7.63 Hz, 1H), 7.83-7.81 (d, J=9 Hz, 2H), 7.63-7.59 (dd, J₁=J₂=7.63 Hz, 1H), 7.56-7.54 (d, J=8.2 Hz, 1H), 7.27-7.26 (d, J=3.72 Hz, 1H), 6.96-6.94 (d, J=9 Hz, 2H), 6.77-6.76 (d, J=3.72 Hz, 1H), 4.14 (s, 2H), 3.65-3.62 (t, J=5.87 Hz, 2H), 3.14-3.12 (dd, J₁=4.89 Hz, J₂=5.09 Hz, 4H), 2.79 (br-s, 1H), 2.66-2.63 (dd, J₁=4.89 Hz, J₂=5.09 Hz, 4H), 2.56-2.53 (t, J=5.87 Hz, 2H) MS (ESI⁺): 454.4 (M+H)⁺

### 2-(3-(2-Chloro-5-methyl-7H-pyrrolo[2,3-d]pyrimidin-4-yl)phenyl)acetonitrile (Cmpd BC)

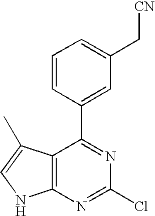

The mixture containing 2,4-dichloro-5-methyl 7H-pyrrolo[2,3-d]pyrimidine (100 mg, 0.495 mmol), 2-(3-(4,4,5,5-tetramethyl-1,3,2-dioxaborolan-2-yl)phenyl)acetonitrile (120 mg, 1 eq) and Pd(PPh₃)₄ (11.44 mg, 0.02 eq) and Na₂CO₃ (0.495 mL, 2M) in Dioxane (5 mL) was heated at 120° C. by microwave for 2 h. Concentration and combiflash (10 g, hexane to EtOAc) afforded 36 mg of EXAMPLE-7078b as off white solid. ¹H-NMR (400 MHz, CDCl₃) 7.66 (m, 2H), 7.52 (m, 2H), 7.14 (s, 1H), 3.86 (s, 2H), 2.08 (s, 3H) ESI-MS: 283.3 (M+H)⁺

2-(3-(2-(3-Fluoro-4-(4-methylpiperazin-1-yl)phenylamino)-5-methyl-7H-pyrrolo[2,3-d]pyrimidin-4-yl)phenyl)acetonitrile (EXAMPLE 43): ¹H-NMR (400 MHz, CD₃OD) 7.90 (dd, J₁=2.35 Hz, J₂=15.5 Hz, 1H), 7.65 (m, 2H), 7.52 (m, 2H), 7.29 (d, J=7.63 Hz, 1H), 6.98 (t, J=9.0 Hz, 1H), 6.89 (s, 1H), 4.02 (s, 2H), 3.06 (s, 4H), 2.63 (s, 4H), 2.35 (s, 3H), 1.96 (s, 3H). ¹⁹F-NMR (400 MHz, CD₃OD) −124.14 ESI-MS: 456.4 (M+H)⁺

2-(3-(2-(3-Fluoro-4-(2-(pyrrolidin-1-yl)ethoxy)phenylamino)-5-methyl-7H-pyrrolo[2,3-d]pyrimidin-4-yl)phenyl)acetonitrile (EXAMPLE 44): ¹HNMR (400 MHz, CD₃OD) 7.88 (dd, J₁=14.08 Hz, J₂=2.15 Hz, 1H), 7.60 (m, 2H), 7.48 (m, 2H), 7.24 (d, J=8.60 Hz, 1H), 6.97 (m, 1H), 6.85 (s, 1H), 4.10 (m, 2H), 3.99 (s, 2H), 2.88 (m, 2H), 2.66 (s, 4H), 1.93 (s, 3H), 1.80 (s, 4H) ¹⁹FNMR (400 MHz, CD₃OD) −134.76, −134.78 ESI-MS: 471.7 (M+H)⁺

### 2-(3-(2-Chloro-5,6-dimethyl-7H-pyrrolo[2,3-d]pyrimidin-4-yl)phenyl)acetonitrile (Cmpd BD)

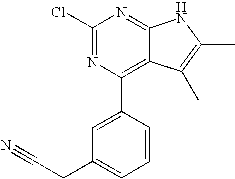

¹H-NMR (400 MHz, CDCl₃) 7.62 (m, 2H), 7.50 (m, 2H), 3.84 (s, 2H), 2.45 (s, 3H), 1.95 (s, 3H) ESI-MS: 297.1 (M+H)⁺

2-(3-(2-(3-Fluoro-4-(4-methylpiperazin-1-yl)phenylamino)-5,6-dimethyl-7H-pyrrolo[2,3-d]pyrimidin-4-yl)phenyl)acetonitrile (EXAMPLE 46): ¹H-NMR (400 MHz, CD₃OD) 7.88 (dd, J₁=2.55 Hz, J₂=5.46 Hz, 1H), 7.61 (m, 2H), 7.51 (m, 2H), 7.26 (dd, J1=1.76 Hz, J2=8.61 Hz, 1H), 6.96 (t, J=9.2 Hz, 1H), 4.00 (s, 2H), 3.05 (s, 4H), 2.62 (s, 4H), 2.33 (s, 3H), 2.28 (s, 3H), 1.84 (s, 3H) ¹⁹F-NMR (400 MHz, CD₃OD) −124.21 ESI: 470.2 (M+H)⁺

2-(3-(2-(4-(4-(2-Hydroxyethyl)piperazin-1-yl)phenylamino)-5-methyl-7H-pyrrolo[2,3-d]pyrimidin-4-yl)phenyl)acetonitrile (EXAMPLE 45): ¹HNMR (400 MHz, CD₃OD) 7.62 (m, 4H), 7.51 (m, 2H), 6.93 (d, J=9.0 Hz, 2H), 6.83 (s, 1H), 4.00 (s, 2H), 3.70 (t, J=6.07 Hz, 2H), 3.11 (m, 4H), 2.68 (m, 4H), 2.57 (t, J=6.07 Hz, 2H), 1.92 (s, 3H). MS: 468.4 (M+H)^{÷}

2-(3-((2-((3-fluoro-4-(4-methylpiperazin-1-yl)phenyl)amino)-5-hydroxy-7H-pyrrolo[2,3-d]pyrimidin-4-yl)amino)phenyl)acetonitrile (EXAMPLE 47): HR-MS: 473.22058 (M+H)⁺Cal: 473.22081

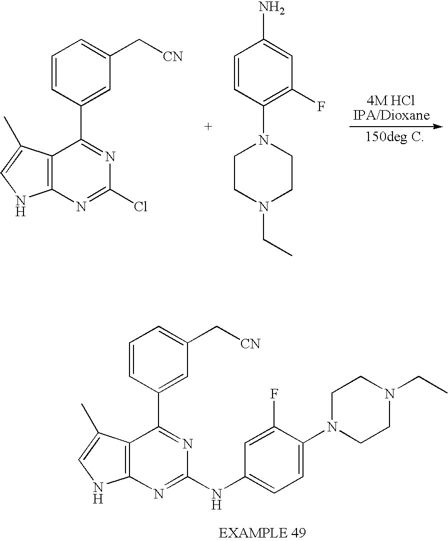

2-(3-(2-((4-(4-ethylpiperazin-1-yl)-3-fluorophenyl)amino)-5-methyl-7H-pyrrolo[2,3-d]pyrimidin-4-yl)phenyl)acetonitrile (EXAMPLE 49): ¹HNMR (400 MHz, CD₃OD) 7.86 (d, J=15.5 Hz, 1H), 7.63 (m, 2H), 7.50 (m, 2H), 7.28 (d, J=8.4 Hz, 1H), 6.97 (t, J=9.0 Hz, 1H), 6.87 (s, 1H), 4.01 (s, 2H), 3.06 (br-s, 4H), 2.65 (br-s, 4H), 2.49 (m, 2H), 1.95 (s, 3H), 1.13 (s, 3H). ESI: 471.4 (M+H)+

Certain pharmaceutical salts were also made of the EXAMPLES:

EXAMPLE 30 Mesylate: Dissolved EXAMPLE 30 in 5 mL chloroform. Added 0.5 mL IPA. Added MsOH acid. Stir for 5 min. To the reaction mixture dropwise over 7 min to 70 mL ether while stirring vigorously. Filtered off solids. While still moist, placed under high vacuum to dry. NMR (400 MHz, CD₃OD) 8.00 (s, 1H), 7.98 (m, 1H), 7.80-7.73 (m, 3H), 7.53 (d, J=3.6 Hz, 1H), 7.35 (d, J=8.4 Hz, 1H), 7.11 (t, J=8.8 Hz, 1H), 6.84 (d, J=4.0 Hz, 1H), 4.11 (s, 2H), 3.66 (d, J=11.6 Hz, 2H), 3.55 (d, J=12.8 Hz, 2H), 3.46 (q, J=7.2 Hz, 2H), 3.26 (m, 2H), 3.13 (m, 2H), 2.71 (s, 6H), 1.39 (t, J=7.0 Hz, 31-1)

EXAMPLE 30HCl: NMR (400 MHz, CD₃OD) 8.04 (s, 1H), 8.00 (m, 1H), 7.82 (d, J=14.4 Hz, 1H), 7.74 (m, 2H), 7.53 (d, J=4.0 Hz, 1H), 7.33 (d, J=8.4 Hz, 1H), 7.15 (t, J=9.2 Hz, 1H), 6.86 (d, J=4.0 Hz, 1H), 4.12 (s, 2H), 3.66 (d, J=11.6 Hz, 2H), 3.58 (d, J=12.8 Hz, 2H), 3.15 (m, 2H), 1.39 (t, J=7.4 Hz, 3H)

EXAMPLE 30HCl and EXAMPLE 30 Tosylate: Another way of making EXAMPLE 30 HCl is through the tosylate:

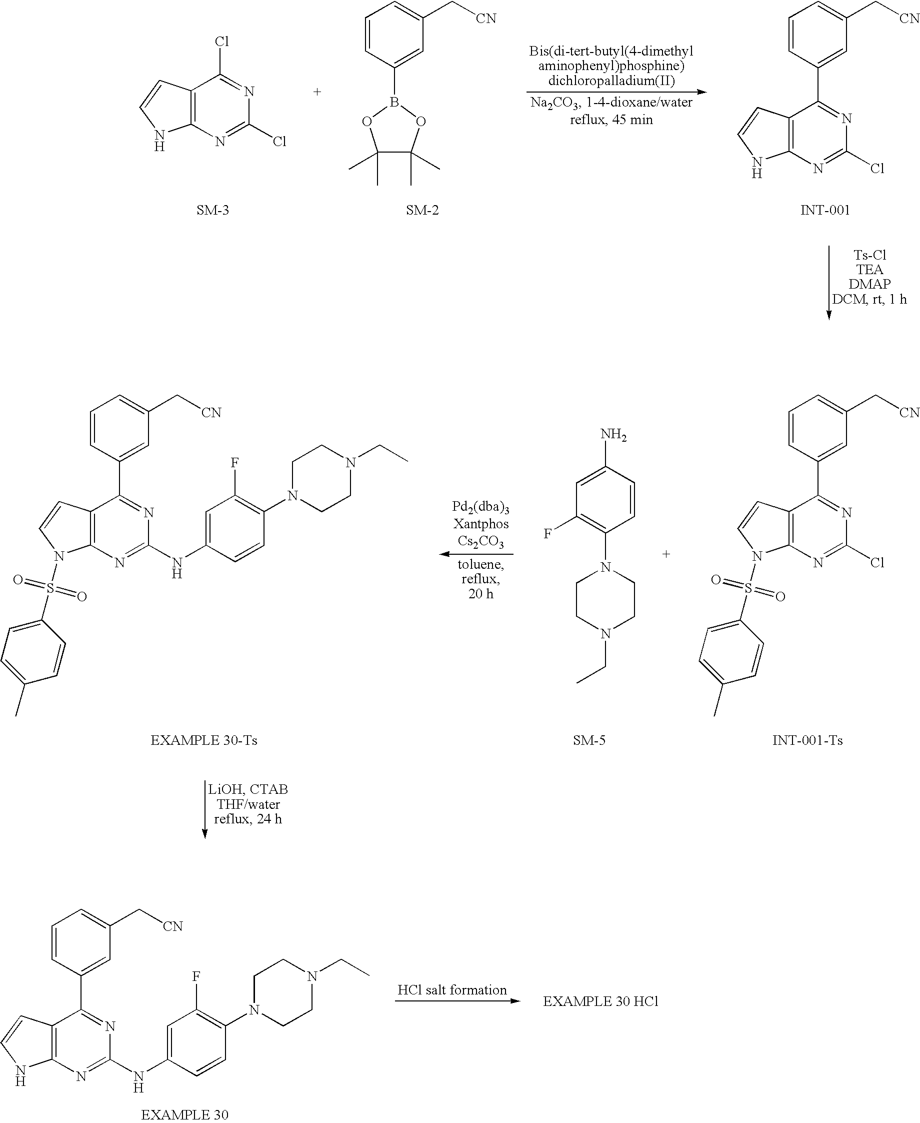

To a mixture of 2,4-dichloro-7H-pyrrolo[2,3-d]pyrimidine (SM-3) and 2-(3-(4,4,5,5-tetramethyl-1,3,2-dioxaborolan-2-yl)phenyl)acetonitrile (SM-2) in dioxane/water mixture was added sodium carbonate followed by bis(di-tert-butyl(4-dimethyl aminophenyl)phosphine) dichloropalladium(II) catalyst as solids at rt. The resulting mixture was heated to reflux for 45 min before being solvent was removed to one third volume and collected separated solid to give 2-(3-(2-chloro-7H-pyrrolo[2,3-d]pyrimidin-4-yl)phenyl)acetonitrile (INT-001).

To a solution of 2-(3-(2-chloro-7H-pyrrolo[2,3-d]pyrimidin-4-yl)phenyl)acetonitrile (INT-001) in dichloromethane was added triethylamine and tosyl chloride followed by catalytic amount of DMAP at rt. The resulting mixture was stirred at rt for 1 h before being solvent was evaporated. To the residue water was added and collected the separated solid at pump to give 2-(3-(2-chloro-7-tosyl-7H-pyrrolo[2,3-d]pyrimidin-4-yl)phenyl)acetonitrile (INT-001-Ts).

To a mixture of 2-(3-(2-chloro-7-tosyl-7H-pyrrolo[2,3-d]pyrimidin-4-yl)phenyl)acetonitrile (INT-001-Ts) and 3-fluoro-4-(4-ethylpiperazin-1-yl)aniline (SM-5) in toluene was added cesium carbonate and xantphos ligand followed by Pd₂(dba)₃ catalyst at rt. The resulting mixture was heated to reflux for 20 h before being solvent was evaporated. To the residue acetonitrile was added and filtered the undissolved solids at pump. The filtrate was concentrated and purified by flash column using MeOH/DCM to give 2-(3-(2-(3-fluoro-4-(4-ethylpiperazin-1-yl)phenylamino)-7-tosyl-7H-pyrrolo[2,3-d]pyrimidin-4-yl)phenyl)acetonitrile (EXAMPLE 30-Ts).

To a solution of 2-(3-(2-(3-fluoro-4-(4-ethylpiperazin-1-yl)phenylamino)-7-tosyl-7H-pyrrolo[2,3-d]pyrimidin-4-yl)phenyl)acetonitrile (EXAMPLE 30-Ts) in THF/water was added LiOH followed by CTAB as catalytic amount at rt. The resulting mixture was heated to reflux for 24 h before being solvent was evaporated. The residue was dissolved in ethyl acetate and washed with water, brine, dried over Na₂SO₄, filtered, and concentrated. The residue was purified by flash column using MeOH/DCM to give 2-(3-(2-(3-fluoro-4-(4-ethylpiperazin-1-yl)phenylamino)-7H-pyrrolo[2,3-d]pyrimidin-4-yl)phenyl)acetonitrile (EXAMPLE 30 free base).

To a solution of 2-(3-(2-(3-fluoro-4-(4-ethylpiperazin-1-yl)phenylamino)-7H-pyrrolo[2,3-d]pyrimidin-4-yl)phenyl)acetonitrile (EXAMPLE 30 free base) in chloroform/IPA mixture was added HCl solution in dioxane at rt. The resulting mixture was stirred for 5 min before being filtered the separated solid and washed with MTBE. After drying at pump for 4 h gave 2-(3-(2-(3-fluoro-4-(4-ethylpiperazin-1-yl)phenylamino)-7H-pyrrolo[2,3-d]pyrimidin-4-yl)phenyl)acetonitrile dihydrochloride salt (EXAMPLE 30.2HCl) in quantitative yield.

Similarly, other EXAMPLES and their salts may be made through the intermediate tosylate salt processed with cesium carbonate and xantphos ligand followed by Pd₂(dba)₃ catalyst. For example, EXAMPLE 43 was made through this tosylate route. The EXAMPLE 43 Tosylate was formed, followed by LiOH and CTAB catalytic treatment to form EXAMPLE 43. The HCl salt was then formed from the free base.

EXAMPLE 38 Mesylate: ¹H NMR (400 MHz, CD₃OD) 8.00 (s, 1H), 7.97 (m, 1H), 7.71 (d, J=4.4 Hz, 2H), 7.64 (d, J=8.4 Hz, 2H), 7.46 (d, J=4.0 Hz, 1H), 7.05 (d, J=8.4 Hz, 2H), 6.78 (d, J=3.2 Hz, 1H), 4.33 (t, J=3.8 Hz, 2H), 4.10 (s, 2H), 3.73 (m, 2H), 3.66 (m, 2H), 3.22 (m, 2H), 2.70 (s, 6H), 2.18 (m, 2H), 2.06 (m, 2H)

EXAMPLE 38HCl: ¹H NMR (400 MHz, CD₃OD) 8.03 (s, 1H), 8.00 (m, 1H), 7.72 (d, J=5.2 Hz, 2H), 7.65 (d, J=8.8 Hz, 2H), 7.46 (d, J=3.6 Hz, 1H), 7.08 (d, J=8.4 Hz, 2H), 6.81 (d, J=3.2 Hz, 1H), 4.35 (t, J=4.8 Hz, 2H), 4.11 (s, 2H), 3.73 (m, 2H), 3.67 (m, 2H), 3.23 (m, 2H), 2.18 (m, 2H), 2.06 (m, 2H)

EXAMPLE 41HCl: ¹H NMR (400 MHz, CD₃OD) 8.02 (s, 1H), 8.00 (m, 1H), 7.74 (d, J=5.2 Hz, 2H), 7.61 (d, J=8.4 Hz, 2H), 7.50 (d, J=3.6 Hz, 1H), 7.09 (t, J=8.4 Hz, 1H), 6.83 (d, J=3.6 Hz, 1H), 4.12 (s, 2H), 3.93 (m, 2H), 3.82 (d, J=13.2 Hz, 2H), 3.73 (d, J=12.8 Hz, 2H), 3.34 (m, 4H), 3.15 (t, J=12.4 Hz, 2H)

EXAMPLE 41 Mesylate: ^{1H} NMR (400 MHz, CD₃OD) 7.99 (s, 1H), 7.96 (m, 1H), 7.75 (m, 2H), 7.62 (d, J=8.8 Hz, 2H), 7.50 (d, J=4.0 Hz, 1H), 7.09 (d, J=8.8 Hz, 1H), 6.82 (d, J=4.0 Hz, 1H), 4.12 (s, 2H), 3.93 (m, 2H), 3.82 (d, J=11.6 Hz, 2H), 3.73 (d, J=12.8 Hz, 2H), 3.34 (m, 3H), 3.14 (m, 2H), 2.69 (s, 6H)

(314) EXAMPLE 39 Mesylate: ¹HNMR (400 MHz, CD₃OD) 8.02 (s, 1H), 7.99 (m, 1H), 7.71 (d, J=4.4 Hz, 2H), 7.63 (m, 1H), 7.45 (bs, 1H), 7.20 (m, 1H), 6.79 (d, J=4.4 Hz, 1H), 4.10 (s, 2H), 3.59 (m, 2H), 3.39 (m, 1H), 3.17 (t, J=12.2, 2H), 2.27 (m, 2H), 2.12 (m, 1H), 1.78 (m, 2H)

EXAMPLE 39 HCl: ¹H NMR (400 MHz, CD₃OD) 8.00 (s, 1H), 7.96 (m, 1H), 7.73 (m, 2H), 7.64 (m, 1H), 7.48 (d, J=3.6 Hz, 1H), 7.23 (d, J=8.4 Hz, 1H), 6.94 (t, J=8.4 Hz, 1H), 6.80 (d, J=3.6 Hz, 1H), 4.11 (s, 2H), 3.59 (d, J=11.8 Hz, 1H), 3.16 (t, J=11.8 Hz, 1H), 2.70 (s, 6H), 2.29 (d, J=12.2 Hz, 2H), 2.12 (m, 1H), 1.77 (q, J=12.2 Hz, 2H)

EXAMPLE 43 HCl: ¹H NMR (400 MHz, CD₃OD) 7.75 (m, 5H), 7.31 (d, J=8.8 Hz, 1H), 7.25 (s, 1H), 7.14 (t, J=9.2 Hz, 1H), 4.10 (s, 2H), 3.59 (m, 4H), 3.29 (m, 2H), 3.16 (m, 2H), 2.97 (s, 3H), 1.96 (s, 3H)

EXAMPLE 40HCl: ¹H NMR (400 MHz, CD₃OD) 8.04 (s, 1H), 8.01 (m, 1H), 7.85 (dd, J=13.2, 2.4 Hz, 1H), 7.75 (d, J=4.4 Hz, 2H), 7.53 (d, J=4.0 Hz, 1H), 7.34 (d, J=8.8 Hz, 1H), 7.25 (t, J=9.0 Hz, 1H), 6.86 (d, J=3.6 Hz, 1H), 4.42 (t, J=5.2 Hz, 2H), 4.12 (s, 2H), 3.77 (m, 2H), 3.70 (t, J=4.6 Hz, 2H), 3.25 (m, 2H), 2.20 (m, 2H), 2.06 (m, 2H)

Like to the EXAMPLES described above, other EXAMPLES may be made similarly according to the following chemical scheme:

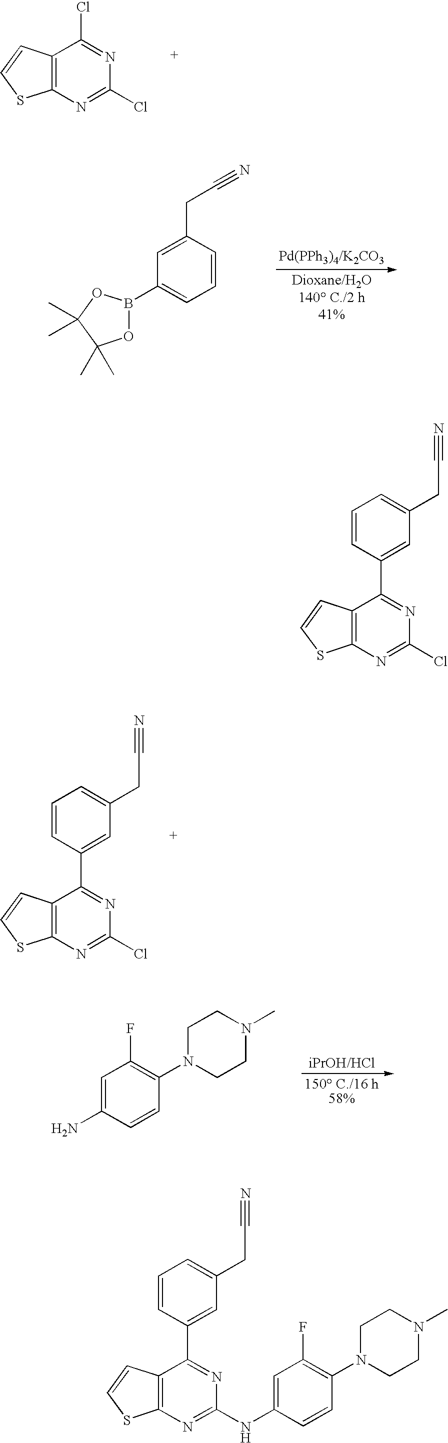

Synthesis of 2-(3-(2-chlorothieno[2,3-d]pyrimidin-4-yl)phenyl)acetonitrile.

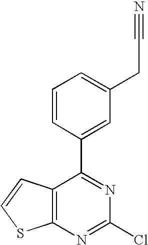

To a solution of 2,4-dichlorothieno[2,3-d]pyrimidine (0.3 g, 1.463 mmol) and 2-(3-(4,4,5,5-tetramethyl-1,3,2-dioxaborolan-2-yl)phenyl)acetonitrile (0.356 g, 1.463 mmol) was dissolved in dioxane (12 mL) and water (2 mL) mixture. Then bubbled with N₂ and added Pb(PPh₃)₄ (85 mg, 0.073 mmol) and K₂CO₃ (0.404 g, 2.93 mmol), and the solution was heated at 140° C. for 120 min. The reaction mixture was dissolved in water (20 mL) and extracted with 50 mL DCM twice. The organic phase was dried with Na₂SO₄ and evaporated. The pure product 2-(3-(2-chlorothieno[2,3-d]pyrimidin-4-yl)phenyl)acetonitrile (0.418 g, 0.595 mmol, 41% yield) was obtained by column chromatography using ethyl acetate/hexane, 2-25% ration solvent system. ¹H-NMR (CDCl₃/400 MHz): 7.91 (m, 2H), 7.60 (m, 4H), 3.87 (s, 2H). MS (ES⁺, m/z): 286.1 (M⁺+1, 80.0).

Synthesis of 2-(3-(2-((3-fluoro-4-(4-methylpiperazin-1-yl)phenyl)amino)thieno[2,3-d]pyrimidin-4-yl)phenyl)acetonitrile. (EXAMPLE 48)

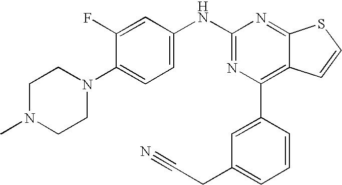

To a solution of 2-(3-(2-chloropyrimidin-4-yl)phenyl)acetonitrile (0.130 g, 0.455 mmol) and 3-fluoro-4-(4-methylpiperazin-1-yl)aniline (0.095 g, 0.455 mmol) was dissolved in iPrOH (5 mL) and 4M HCl in dioxane (0.227 mL). The solution was heated at 150° C. for 16 h. TLC indicated the reaction is very clean. The organic phase was neutralized with NaHCO₃ and dried with Na₂SO₄. The pure product 2-(3-(2-((3-fluoro-4-(4-methylpiperazin-1-yl)phenyl)amino)thieno[2,3-d]pyrimidin-4-yl)phenyl)acetonitrile (EXAMPLE 48) was obtained by column chromatography using methanol/DCM, 0-5% ration solvent system. ¹H-NMR (CDCl₃/400 MHz): 7.87 (m, 2H), 7.69 (d, J=14.4 Hz, 1H), 7.55 (t, J=7.6 Hz, 1H), 7.49 (d, J=8.0 Hz, 1H), 7.34 (d, J=6.0 Hz, 1H), 7.22 (d, J=6.0 Hz, 1H), 7.17 (d, J=6.0 Hz, 1H), 6.94 (t, J=9.2 Hz, 1H), 3.85 (s, 2H), 3.10 (s, 4H), 2.62 (s, 4H), 2.36 (s, 3H). MS (ES⁺, m/z): 459.3 (M⁺+1, 100.0).

Any U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing it will be appreciated that, although specific embodiments of the invention have been described herein for purposes of illustration, various modifications may be made without deviating from the spirit and scope of the invention.

We claim:

1. A compound according to Formula (I): 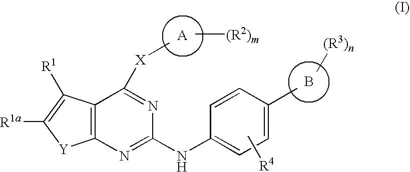
and pharmaceutically acceptable salts thereof, wherein:
X is —NH—, S, or a direct bond;
Y is —NH— or S;
A is aryl or hetaryl;
B is —O—C₁₋₄alkyl-N(C₀₋₄alkyl)(C₀₋₄alkyl); or C₁₋₄alkyl optionally substituted by —CN; or
B is hetcyclyl, —C(O)-hetcyclyl, —NH-hetcyclyl, or —O—C₀₋₄alkyl-hetcyclyl;
R^{1a} is C₀₋₄alkyl;
R¹ is halo, —CN, —OH, C₀₋₄alkyl, halo substituted C₁₋₄alkyl, —COOH, or —CONH₂;
R² in each instance independently is —CN, halo, C₀₋₄alkyl, —O—C₁₋₄alkyl, —O—C₁₋₄haloalkyl, or —N(R^{b})(R^{a}); or C₁₋₄alkyl optionally substituted by halo, —CN, —O—C₁₋₄alkyl, or —O—C₁₋₄haloalkyl; R^{a} and R^{b} each independently in each case is C₀₋₄alkyl, or —C(O)—C₃₋₆cycloalkyl;
R³ in each instance independently is —CN, C₀₋₄alkyl, halo, C₀₋₄alkyl-N(C₀₋₄alkyl)(C₀₋₄alkyl), C₃₋₈cycloalkyl, —S(O)₂—CH₃, or —C(O)—O—C₁₋₄alkyl-aryl; or C₁₋₄alkyl optionally substituted with 1-6 independent halo or OH substituents;
R⁴ is C₀₋₄alkyl, halo, or halo substituted C₁₋₄alkyl;
m is 0, 1, 2 or 3; and
n is 0, 1, 2, or 3; with the proviso that the compound is not

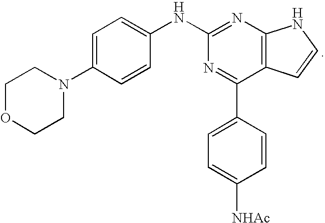

2. The compound according to claim 1, or a pharmaceutically acceptable salt thereof, wherein Y is —NH—.

3. The compound according to claim 2, or a pharmaceutically acceptable salt thereof, wherein X is —NH—.

4. The compound according to claim 3, or a pharmaceutically acceptable salt thereof, wherein A is aryl.

5. The compound according to claim 4, or a pharmaceutically acceptable salt thereof, wherein B is C₁₋₄alkyl optionally substituted by —CN.

6. The compound according to claim 4, or a pharmaceutically acceptable salt thereof, wherein B is —C(O)-hetcyclyl.

7. The compound according to claim 4, or a pharmaceutically acceptable salt thereof, wherein B is hetcyclyl.

8. The compound according to claim 4, or a pharmaceutically acceptable salt thereof, wherein B is —NH-hetcyclyl.

9. The compound according to claim 4, or a pharmaceutically acceptable salt thereof, wherein B is —O—C₁₋₄alkyl-N(C₀₋₄alkyl)(C₀₋₄alkyl).

10. The compound according to claim 4, or a pharmaceutically acceptable salt thereof, wherein B is or —O—C₀₋₄alkyl-hetcyclyl.

11. The compound according to claim 3, or a pharmaceutically acceptable salt thereof, wherein A is phenyl, and B is —C(O)-hetcyclyl.

12. The compound according to claim 3, or a pharmaceutically acceptable salt thereof, wherein A is phenyl, and B is hetcyclyl.

13. The compound according to claim 3, or a pharmaceutically acceptable salt thereof, wherein A is phenyl, and B is C₁₋₄alkyl optionally substituted by —CN.

14. The compound according to claim 3, or a pharmaceutically acceptable salt thereof, wherein A is phenyl, B is —O—C₁₋₄alkyl-N(C₀₋₄alkyl)(C₀₋₄alkyl.

15. The compound according to claim 3, or a pharmaceutically acceptable salt thereof, wherein A is phenyl, B is —NH-hetcyclyl.

16. The compound according to claim 3, or a pharmaceutically acceptable salt thereof, wherein A is phenyl, B is —O—C₀₋₄alkyl-hetcyclyl.

17. The compound according to claim 2, or a pharmaceutically acceptable salt thereof, wherein X is a direct bond.

18. The compound according to claim 17, or a pharmaceutically acceptable salt thereof, wherein A is aryl.

19. The compound according to claim 18, or a pharmaceutically acceptable salt thereof, wherein B is C₁₋₄alkyl optionally substituted by —CN.

20. The compound according to claim 18, or a pharmaceutically acceptable salt thereof, wherein B is —C(O)-hetcyclyl.

21. The compound according to claim 18, or a pharmaceutically acceptable salt thereof, wherein B is hetcyclyl.

22. The compound according to claim 18, or a pharmaceutically acceptable salt thereof, wherein B is —O—C₁₋₄alkyl-N(C₀₋₄alkyl)(C₀₋₄alkyl).

23. The compound according to claim 18, or a pharmaceutically acceptable salt thereof, wherein B is —NH-hetcyclyl.

24. The compound according to claim 18, or a pharmaceutically acceptable salt thereof, wherein B is —O—C₀₋₄alkyl-hetcyclyl.

25. The compound according to claim 17, or a pharmaceutically acceptable salt thereof, wherein A is phenyl, and B is —C(O)-hetcyclyl.

26. The compound according to claim 17, or a pharmaceutically acceptable salt thereof, wherein A is phenyl, and B is hetcyclyl.

27. The compound according to claim 17, or a pharmaceutically acceptable salt thereof, wherein A is phenyl, and B is C₁₋₄alkyl optionally substituted by CN.

28. The compound according to claim 17, or a pharmaceutically acceptable salt thereof, wherein A is phenyl, and B is —O—C₁₋₄alkyl-N(C₀₋₄alkyl)(C₀₋₄alkyl).

29. The compound according to claim 17, or a pharmaceutically acceptable salt thereof, wherein A is phenyl, and B is —NH-hetcyclyl.

30. The compound according to claim 17, or a pharmaceutically acceptable salt thereof, wherein A is phenyl, and B is —O—C₀₋₄alkyl-hetcyclyl.

31. The compound according to claim 1, or a pharmaceutically acceptable salt thereof, wherein Y is —S—.

32. The compound according to claim 31, or a pharmaceutically acceptable salt thereof, wherein X is a direct bond.

33. The compound according to claim 32, or a pharmaceutically acceptable salt thereof, wherein A is aryl.

34. The compound according to claim 33, or a pharmaceutically acceptable salt thereof, wherein B is C₁₋₄alkyl optionally substituted by —CN.

35. The compound according to claim 1, consisting of 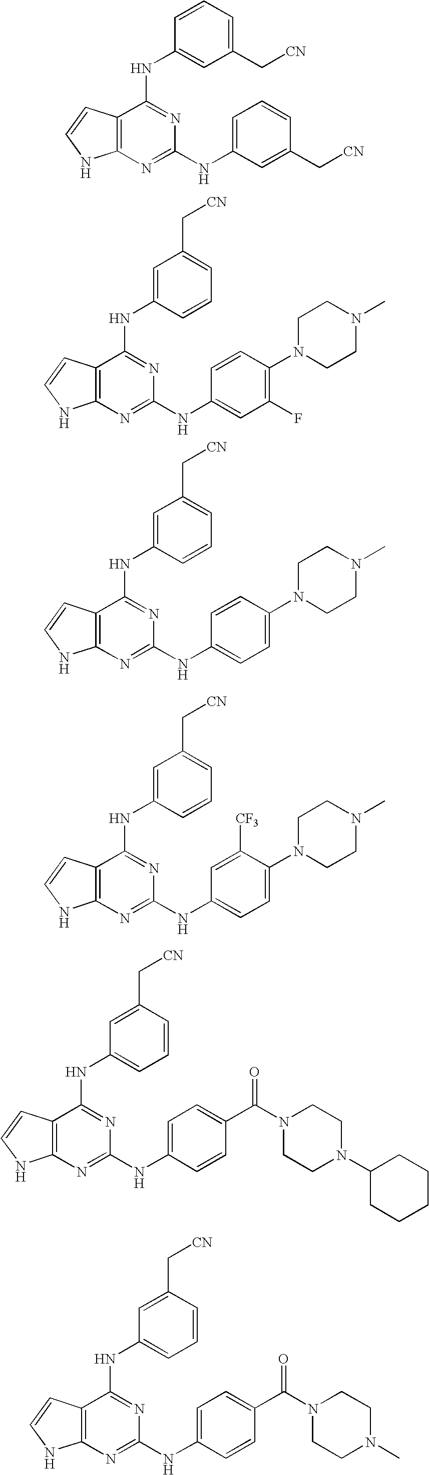 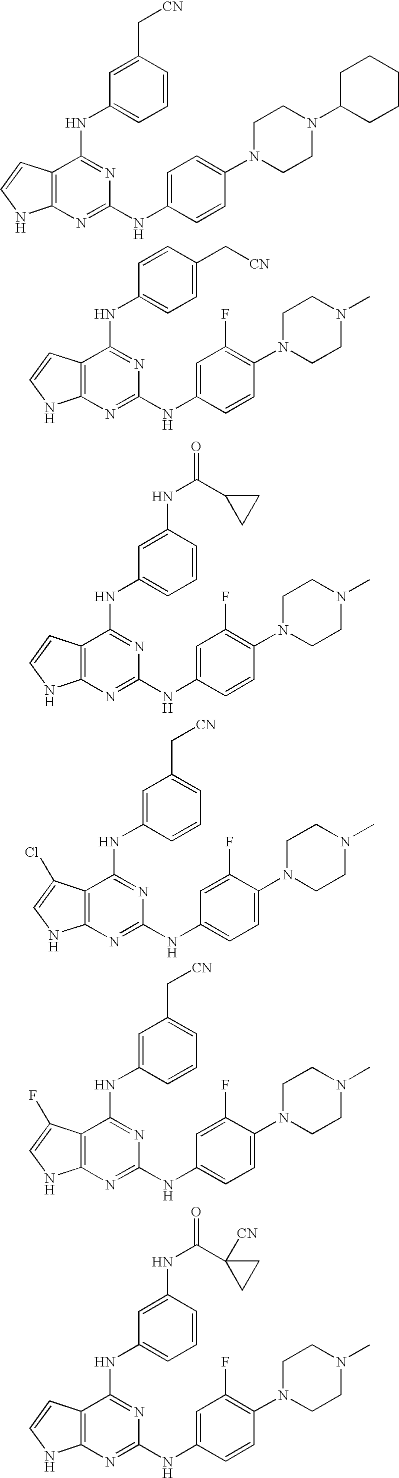 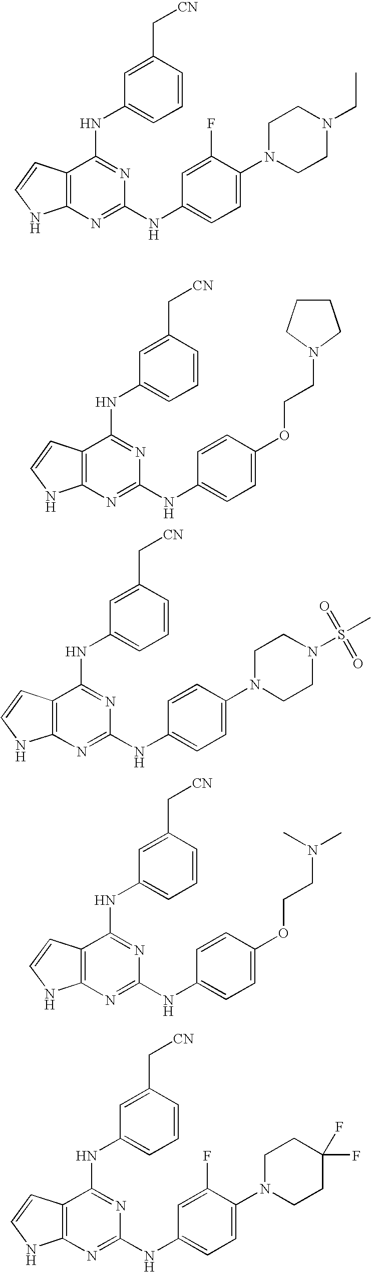 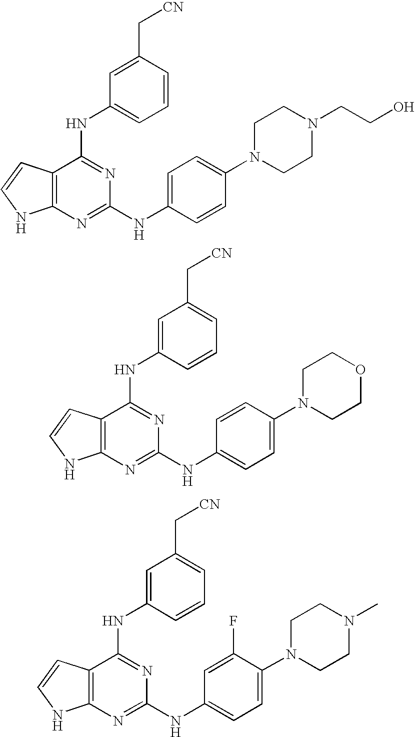
or a stereoisomer, prodrug, or pharmaceutically acceptable salt thereof.

36. The compound according to claim 1, consisting of 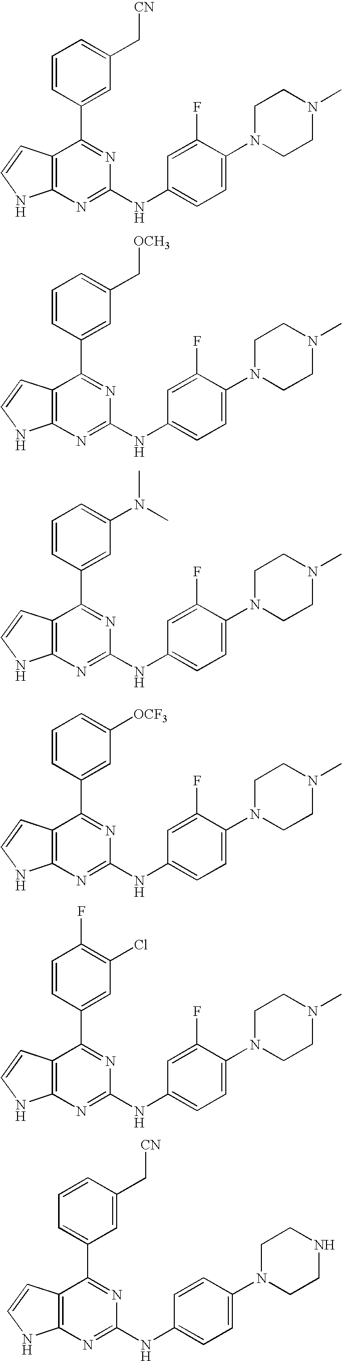 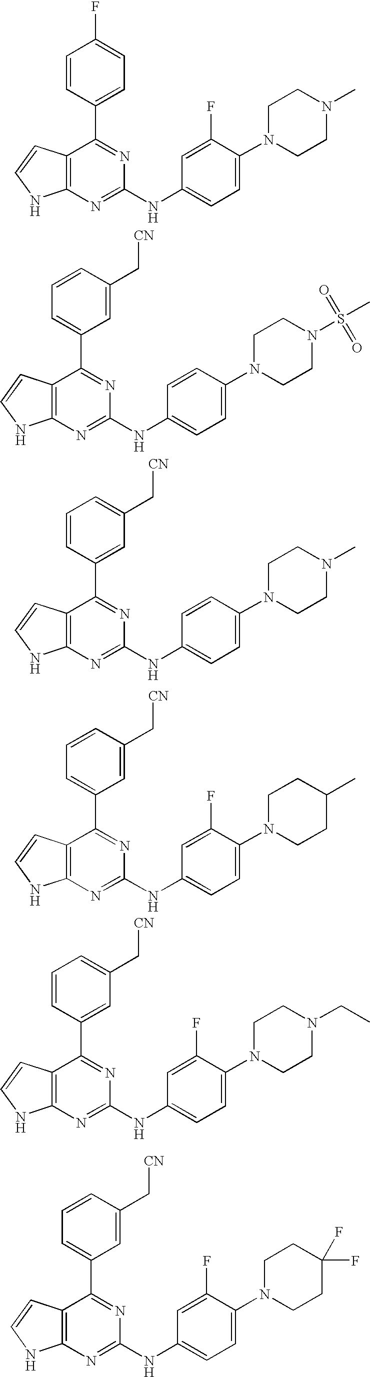 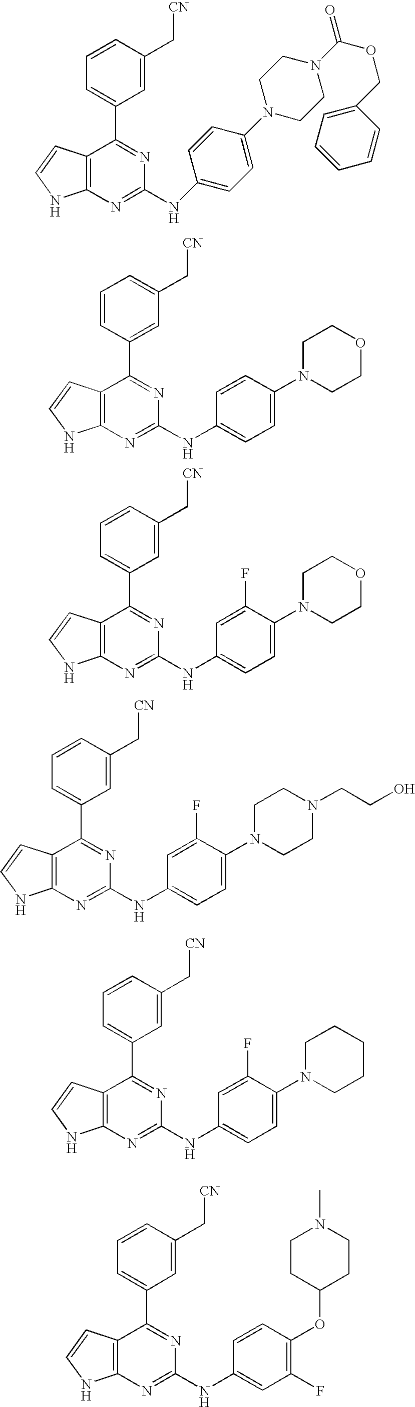 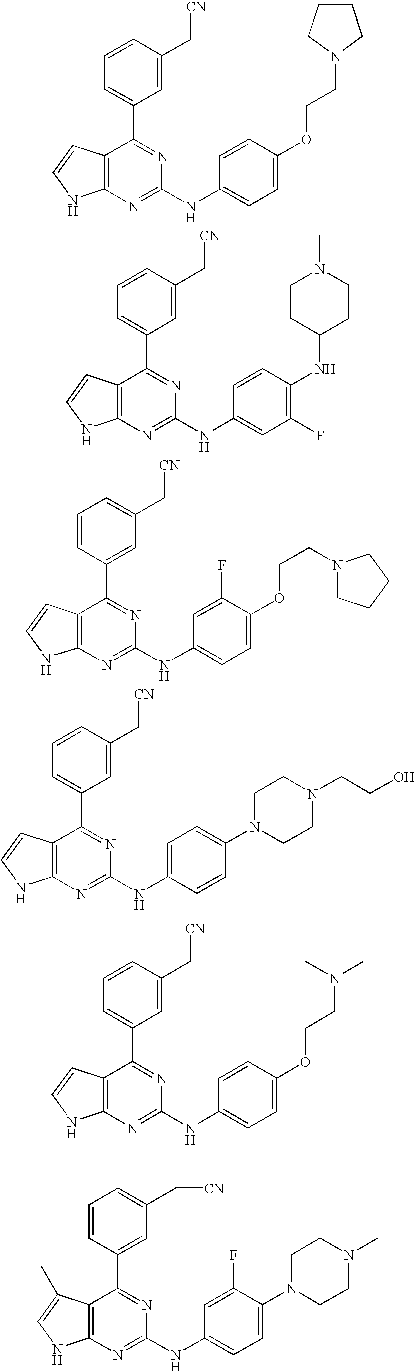 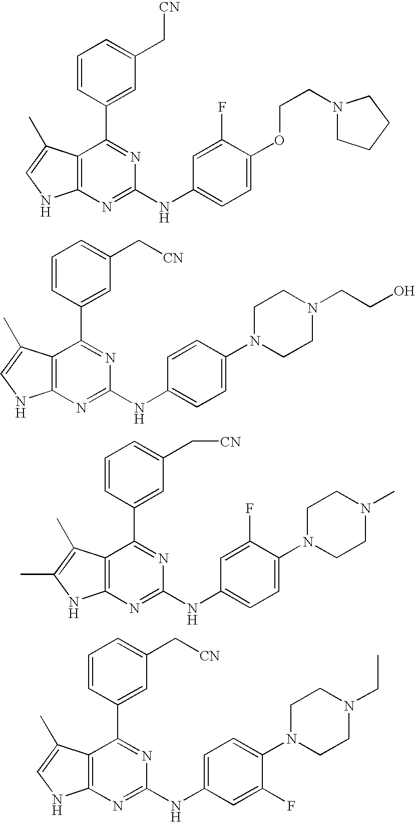
or a stereoisomer, prodrug, or pharmaceutically acceptable salt thereof.

37. The compound according to claim 1, consisting of 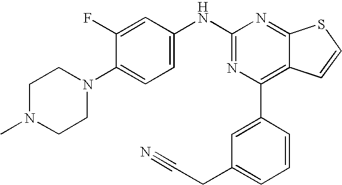
or a stereoisomer, prodrug, or pharmaceutically acceptable salt thereof.

38. A method of treating cancer or hyperproliferative disorders by administering an effective amount of the compound according to claim 1.

39. The method of claim 38, wherein the cancer is of colon, breast, stomach, prostate, pancreas, or ovarian tissue.

40. A method of treating lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter, renal cell carcinoma, carcinoma of the renal pelvis, pediatric malignancy, neoplasms of the central nervous system, primary CNS lymphoma, spinal axis tumors, medulloblastoma, brain stem gliomas, pituitary adenomas, Barrett's esophagus, pre-malignant syndrome, neoplastic cutaneous disease, psoriasis, mycoses fungoides, benign prostatic hypertrophy, diabetic retinopathy, retinal ischemia, and retinal neovascularization, hepatic cirrhosis, angiogenesis, cardiovascular disease, atherosclerosis, immunological disease, autoimmune disease, or renal disease by administering to one in need of such treatment an effective amount of the compound according to claim 1.

41. A composition comprising a compound according to claim 1 and a pharmaceutically acceptable excipient.

42. A method of treatment or prevention of Castleman's disease, atherosclerosis, coronary artery disease, peripheral edema, peripheral vascular disease, glaucoma, and wet or dry age-related macular degeneration (AMD), asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration by administering to one in need of such treatment or prevention an effective amount, or a prophylactically effective amount, of the compound according to claim 1.
